(19) United States
(12) Patent Application Publication
(10) Pub. No.: US 2024/0317848 A1
(43) Pub. Date: Sep. 26, 2024

(54) MODULATING THE EFFECTS OF GAMMA-C-CYTOKINE SIGNALING FOR THE TREATMENT OF ALOPECIA AND ALOPECIA ASSOCIATED DISORDERS

(71) Applicant: BIONIZ THERAPEUTICS, INC., LA JOLLA, CA (US)

(72) Inventors: Yutaka Tagaya, Rockville, MD (US); Nazli Azimi, San Juan Capistrano, CA (US)

(21) Appl. No.: 18/670,579

(22) Filed: May 21, 2024

Related U.S. Application Data
(62) Division of application No. 16/863,914, filed on Apr. 30, 2020, now Pat. No. 12,030,936.
(60) Provisional application No. 62/842,846, filed on May 3, 2019.

(51) Int. Cl. C07K 16/24; A61K 47/64; A61K 47/68; A61P 17/14

(52) U.S. Cl. CPC C07K 16/246; A61K 47/643; A61K 47/6811; A61P 17/14; C07K 16/247

(57) ABSTRACT

The [symbol] c-family cytokines, Interleukin-2 (IL-2), Interleukin-4 (IL-4), Interleukin-7 (IL-7), Interleukin-9 (IL-9), Interleukin-15 (IL-15), and Interleukin-21 (IL-21), are associated with important human diseases, such as alopecia and alopecia associated disorders. Compositions, methods, and kits to modulate signaling by at least one [symbol] c-cytokine family member for inhibiting, ameliorating, reducing a severity of, treating, delaying the onset of, or preventing at least one alopecia related disorder are described.

### CROSS REFERENCE TO RELATED APPLICATIONS

This application is a Divisional Application of U.S. patent application Ser. No. 16/863,914, filed Apr. 30, 2020, which claims the benefit of priority to U.S. Provisional Patent Application No. 62/842,846, filed May 3, 2019. The foregoing application is fully incorporated herein by reference for all purposes.

### SEQUENCE LISTING IN ELECTRONIC FORMAT

The present application is being filed along with a Electronic Sequence Listing as an XML file via Patent Center. The Electronic Sequence Listing is provided as a file entitled BION012ASEQLIST.xml, created and last saved on May 19, 2024, which is 79,934 bytes in size. The information in the electronic format of the Sequence Listing is incorporated herein by reference in its entirety in accordance with 35 U.S.C. § 1.52(e).

### BACKGROUND

### Field

The present embodiments relate to inhibiting, ameliorating, reducing a severity of, treating, delaying the onset of, or preventing autoimmune diseases such as alopecia, and alopecia associated disorders using one or more therapeutic compounds by modulating the signaling by at least one [symbol] c-cytokine family member.

### Description of the Related Art

Cytokines are a diverse group of soluble factors that mediate various cell functions, such as, growth, functional differentiation, and promotion or prevention of programmed cell death (apoptotic cell death). Cytokines, unlike hormones, are not produced by specialized glandular tissues, but can be produced by a wide variety of cell types, such as epithelial, stromal or immune cells.

The γc-family cytokines are a group of mammalian cytokines that are mainly produced by epithelial, stromal and immune cells and control the normal and pathological activation of a diverse array of lymphocytes. These cytokines are critically required for the early development of T cells in the thymus as well as their homeostasis in the periphery.

### SUMMARY

In some embodiments, a composition comprises a therapeutic compound in an amount sufficient to modulate signaling by at least one [symbol] c-cytokine family member, thereby inhibiting, ameliorating, reducing a severity of, treating, delaying the onset of, or preventing at least one alopecia related disorder, and a pharmaceutically acceptable carrier.

In some embodiments of the composition, the at least one alopecia related disorder is selected from the group consisting of alopecia, pemphigus, pemphigoid, psoriasis, vitiligo, graft-versus-host disease, and immune-mediated hair loss.

In some embodiments of the composition, the at least one [symbol] c-cytokine family member is selected from the group consisting of IL-2, IL-4, IL-7, IL-9, IL-15 and IL-21.

In some embodiments of the composition, the therapeutic compound is at least one of a [symbol] c cytokine antagonist peptide, a [symbol] c cytokine antagonist peptide derivative, anti-CD8 antibody, anti-IL-2 antibody, anti-IL-15 antibody, anti-NKG2A antibody, or a combination thereof.

In some embodiments of the composition, the [symbol] c cytokine antagonist peptide comprises a partial sequence of a [symbol] c-box D-helix region of each of at least two [symbol] c-cytokine family members.

In some embodiments of the composition, the partial sequence comprises consecutive blocks of at least 5 amino acids of the [symbol] c-box D-helix region of each of at least two [symbol] c-cytokine family members.

In some embodiments of the composition, the partial sequence comprises consecutive blocks of 1-10 amino acids of the [symbol] c-box D-helix region of each of at least two [symbol] c-cytokine family members.

In some embodiments of the composition, the [symbol] c-box D-helix region of each of at least two [symbol] c-cytokine family members is selected from the group consisting of IL-15, IL-2, IL-21, IL-4, IL-9, and IL-7.

In some embodiments of the composition, the [symbol] c cytokine antagonist peptide comprises 11 to 50 amino acids.

In some embodiments of the composition, the [symbol] c cytokine antagonist peptide further comprises a conjugate at the N-termini, C-termini, side residues, or a combination thereof.

In some embodiments of the composition, the conjugate comprises one or more additional moieties selected from the group consisting of bovine serum albumin (BSA), albumin, Keyhole Limpet Hemocyanin (KLH), Fc region of IgG, a biological protein that functions as scaffold, an antibody against a cell-specific antigen, a receptor, a ligand, a metal ion, and Poly Ethylene Glycol (PEG).

In some embodiments of the composition, the [symbol] c cytokine antagonist peptide further comprises a signal peptide.

In some embodiments of the composition, the [symbol] c cytokine antagonist peptide comprises the amino acid sequence D/E-F-L-E/Q/N-S/R-X-I/K-X-L/I-X-Q (SEQ ID NO: 2), wherein X denotes any amino acid.

In some embodiments of the composition, the [symbol] c cytokine antagonist peptide derivative shares at least about 50% identity with a peptide of SEQ ID NO: 2.

In some embodiments of the composition, the [symbol] c cytokine antagonist peptide derivative shares at least about 90% identity with a peptide of SEQ ID NO: 2.

In some embodiments of the composition, the [symbol] c cytokine antagonist peptide derivative shares at least about 95% identity with a peptide of SEQ ID NO: 2.

In some embodiments of the composition, the [symbol] c cytokine antagonist peptide comprises a sequence of SEQ ID NO: 1 (BNZ- [symbol] )

In some embodiments of the composition, the [symbol] c cytokine antagonist peptide and the [symbol] c antagonist peptide derivative have similar physico-chemical properties but distinct biological activities.

In some embodiments of the composition, the [symbol] c cytokine antagonist peptide derivative shares at least about 50% identity with a peptide of SEQ ID NO: 1.

In some embodiments of the composition, the [symbol] c cytokine antagonist peptide derivative shares at least about 90% identity with a peptide of SEQ ID NO: 1.

In some embodiments of the composition, the [symbol] c cytokine antagonist peptide derivative shares at least about 95% identity with a peptide of SEQ ID NO: 1.

In some embodiments of the composition, the pharmaceutically acceptable carrier is formulated for topical, oral, and/or parenteral delivery.

In some embodiments of the composition, the pharmaceutically acceptable carrier is formulated for topical delivery.

In some embodiments of the composition, the pharmaceutically acceptable carrier is formulated for oral delivery.

In some embodiments of the composition, the pharmaceutically acceptable carrier is formulated for parenteral delivery.

In some embodiments, a method of inhibiting, ameliorating, reducing a severity of, treating, delaying the onset of, or preventing at least one alopecia related disorder comprises administering one or more of the compositions provided herein to a subject in need thereof, thereby inhibiting, ameliorating, reducing a severity of, treating, delaying the onset of, or preventing the at least one alopecia related disorder.

In some embodiments of the method of inhibiting, ameliorating, reducing a severity of, treating, delaying the onset of, or preventing at least one alopecia related disorder, the at least one alopecia related disorder is selected from the group consisting of alopecia, pemphigus, pemphigoid, psoriasis, vitiligo, graft-versus-host disease, and immune-mediated hair loss.

In some embodiments, a method of designing a [symbol] c-cytokine antagonist peptide and/or a derivative thereof configured to modulate and/or block signaling by at least one [symbol] c-cytokine family member that inhibits, ameliorates, reduces a severity of, treats, delays the onset of, or prevents at least one alopecia related disorder comprises the steps of using a computer to obtain from an amino acid sequence database amino acid sequences of at least one a [symbol] c-cytokine family member, assembling a [symbol] c cytokine antagonist peptide and/or a derivative thereof based on a sequence of the at least one [symbol] c-cytokine family member, wherein the [symbol] c cytokine antagonist peptide and/or the derivative thereof modulates and/or blocks signaling by the at least one [symbol] c-cytokine family member.

In some embodiments of the method of designing a [symbol] c-cytokine antagonist peptide and/or a derivative thereof, the at least one [symbol] c-cytokine family member is selected from the group consisting of IL-2, IL-4, IL-7, IL-9, IL-15 and IL-21.

In some embodiments of the method of designing a [symbol] c-cytokine antagonist peptide and/or a derivative thereof, the [symbol] c cytokine antagonist peptide comprises a partial sequence of a [symbol] c-box D-helix region of each of at least two [symbol] c-cytokine family members.

In some embodiments of the method of designing a [symbol] c-cytokine antagonist peptide and/or a derivative thereof, the sequence comprises consecutive blocks of at least 5 amino acids of the [symbol] c-box D-helix region of each of at least two [symbol] c-cytokine family members.

In some embodiments of the method of designing a [symbol] c-cytokine antagonist peptide and/or a derivative thereof, the sequence comprises consecutive blocks of 1-10 amino acids of the [symbol] c-box D-helix region of each of at least two [symbol] c-cytokine family members.

In some embodiments of the method of designing a [symbol] c-cytokine antagonist peptide and/or a derivative thereof, the [symbol] c-box D-helix region of each of at least two [symbol] c-cytokine family members is selected from the group consisting of IL-15, IL-2, IL-21, IL-4, IL-9, and IL-7

In some embodiments of the method of designing a [symbol] c-cytokine antagonist peptide and/or a derivative thereof, the [symbol] c cytokine antagonist peptide comprises 11 to 50 amino acids.

In some embodiments of the method of designing a [symbol] c-cytokine antagonist peptide and/or a derivative thereof, the [symbol] c cytokine antagonist peptide further comprises a conjugate at the N-termini, C-termini, side residues, or a combination thereof.

In some embodiments of the method of designing a [symbol] c-cytokine antagonist peptide and/or a derivative thereof, the [symbol] c cytokine antagonist peptide further comprises a signal peptide.

In some embodiments of the method of designing a [symbol] c-cytokine antagonist peptide and/or a derivative thereof, the [symbol] c cytokine antagonist peptide comprises the amino acid sequence D/E-F-L-E/Q/N-S/R-X-I/K-X-L/I-X-Q (SEQ ID NO: 2), wherein X denotes any amino acid.

In some embodiments of the method of designing a [symbol] c-cytokine antagonist peptide and/or a derivative thereof, the [symbol] c cytokine antagonist peptide derivative shares at least about 50% identity with a peptide of SEQ ID NO: 2.

In some embodiments of the method of designing a [symbol] c-cytokine antagonist peptide and/or a derivative thereof, the [symbol] c cytokine antagonist peptide derivative shares at least about 90% identity with a peptide of SEQ ID NO: 2.

In some embodiments of the method of designing a [symbol] c-cytokine antagonist peptide and/or a derivative thereof, the [symbol] c cytokine antagonist peptide derivative shares at least about 95% identity with a peptide of SEQ ID NO: 2.

In some embodiments of the method of designing a [symbol] c-cytokine antagonist peptide and/or a derivative thereof, the [symbol] c cytokine antagonist peptide comprises a sequence of SEQ ID NO: 1 (BNZ- [symbol] )

In some embodiments of the method of designing a [symbol] c-cytokine antagonist peptide and/or a derivative thereof, the [symbol] c cytokine antagonist peptide derivative shares at least about 50% identity with a peptide of SEQ ID NO: 1.

In some embodiments of the method of designing a [symbol] c-cytokine antagonist peptide and/or a derivative thereof, the [symbol] c cytokine antagonist peptide derivative shares at least about 90% identity with a peptide of SEQ ID NO: 1.

In some embodiments of the method of designing a [symbol] c-cytokine antagonist peptide and/or a derivative thereof, the [symbol] c cytokine antagonist peptide derivative shares at least about 95% identity with a peptide of SEQ ID NO: 1.

In some embodiments of the method of designing a Ic-cytokine antagonist peptide and/or a derivative thereof, the c cytokine antagonist peptide and the derivative thereof have similar physico-chemical properties but distinct biological activities.

In some embodiments, a kit for inhibiting, ameliorating, reducing a severity of, treating, delaying the onset of, or preventing at least one alopecia related disorder comprises one or more of the compositions provided herein.

In some embodiments of the kit, the at least one alopecia related disorder is selected from the group consisting of alopecia, pemphigus, pemphigoid, psoriasis, vitiligo, graft-versus-host disease, and immune-mediated hair loss.

### BRIEF DESCRIPTION OF THE DRAWINGS

FIG. 1A shows an alignment of the D-helix region of human [symbol] c-cytokine family members.

FIG. 1B depicts the γc-box (SEQ ID NO: 9) and IL-2/IL-15 box (SEQ ID NO: 10) motifs which give rise to the consensus sequence around the D-helix region of the [symbol] c-cytokines.

FIG. 2 depicts a diagramed representation of the biochemical properties of amino acids.

FIG. 3A shows inhibition of IL-15, and IL-9 activity by BNZ- [symbol] in a PT-18 proliferation assay.

FIG. 3B shows a proliferation assay of CTLL-2 cells grown in the presence of IL-2 or IL-15 and 0, 0.1, 1 or 10 [symbol] M BNZ [symbol] [symbol] .

FIG. 4 shows inhibition of IL-15-mediated tyrosine-phosphorylation of STAT5 by BNZ [symbol] [symbol] .

FIG. 5 shows circulating levels of the human cytokines IL-2, IL-15, and IFNγ following huPBMC transplant to NSG mice.

FIG. 6A shows that human CD8+ T-cells from a representative NSG mouse 4-weeks post-huPBMC transplantation fully express NKG2D (CD314).

FIG. 6B shows the expansion of NKG2A+ human CD8+ T-cells (boxed) in a representative NSG mouse from 1-week to 4-weeks post-huPBMC transplantation.

FIG. 7A shows specific depletion of human CD8+ T-cells following injection of an anti-CD8 antibody in a representative NSG mouse that was 4-weeks post-huPBMC transplantation. Post-anti-CD8 AB graph is 8-days post antibody injection.

FIG. 7B shows the average recovery of body weight in grams in days following anti-CD8 antibody-mediated human CD8+ T-cell depletion in three NSG mice that were antibody treated at 4-weeks post-huPBMC transplantation.

FIG. 7C shows the regrowth of body hair following anti-CD8 antibody-mediated human CD8+ T-cell depletion in a representative NSG mouse 14-days post antibody injection and 42-days post-huPBMC transplantation.

FIG. 8 shows the positive phosphorylation of Jak3 and STAT5 in NKG2A+(+), but not NKG2A−(−) CD8+ T-cells isolated from representative NSG mouse 4-weeks post-huPBMC transplantation indicative of constitutive activation of [symbol] c-cytokine signaling.

FIG. 9A shows the positive correlation between the expansion of NKG2A+ human CD8+ T-cells and the levels of inflammatory cytokine IFN [symbol] [symbol] and the [symbol] c-cytokines IL-2 and IL-15 from three representative humanized NSG mice over the course of 1-week to 6-weeks post-huPBMC transplantation.

FIG. 9B shows the effective depletion of human NKG2A+ CD8+ T-cells via administration of an anti-NKG2A antibody twice per week in three representative humanized NSG mice at 3-to 5-weeks post-huPBMC transplantation results in an improvement of GvHD symptoms such as loss in body weight, and a significant reduction of the [symbol] c-cytokines IL-2, IL-15, and the inflammatory cytokine IFN

[symbol]

[symbol]

[symbol]

FIG. 10A shows the reversal of immune-mediated hair loss by BNZ-γ in a representative NSG mouse. Time points: Day −30 is prior to huPBMC transplantation. Day 0 is 4-wecks post-huPBMC transplantation. Day 7 is 5-weeks post-huPBMC transplantation and 1 week into a twice weekly BNZ-γ dosing regimen for a treatment duration of two weeks. Day 21 is 7-weeks post-huPBMC transplantation and 1 week following completion of a twice weekly BNZ-γ dosing regimen for a treatment duration of two weeks. Day 30 is just over 8-weeks post-huPBMC transplantation and just over 2 weeks following completion of a twice weekly BNZ-γ dosing regimen for a treatment duration of two weeks.

FIG. 10B shows a comparison of serum concentrations of the of circulating human inflammatory cytokines IL-6 and IFN [symbol] [symbol] in two representative NSG mice 6-weeks post-huPBMC transplantation with and without (PBS control) completion of a twice weekly BNZ-γ dosing regimen for a treatment duration of two weeks. The results were statistically significant (***), p<0.001.

FIG. 11A shows survival curves of humanized NSG mice that began therapeutic treatment 35-days post-huPBMC transplantation with PBS control (untreated), anti-IL-2 antibody, anti-IL-15 antibody, combination anti-IL-2 and anti-IL-15 antibody, and BNZ-γ.

FIG. 11B shows a comparison of the level of hair regrowth in a representative NSG mouse from each of the treatment groups: PBS control, anti-IL-2 antibody (AB), anti-IL-15 AB, combination anti-IL-2 and anti-IL-15 AB, and BNZ-γ following the completion of a four-week treatment regimen on NSG mice at 35-days post-huPBMC transplantation.

FIG. 11C shows a comparison of average serum concentrations of the of circulating human inflammatory cytokines IL-6 and IFN [symbol] [symbol] from each of the treatment groups: PBS control, anti-IL-2 antibody (Ab), anti-IL-15 Ab, combination anti-IL-2 and anti-IL-15 Ab, and BNZ-γ following the completion of a four-week treatment regimen on NSG mice at 35-days post-huPBMC transplantation.

FIG. 12 shows immuno-stained skin tissue for human CD8+ T-cells from humanized NSG mice 3-weeks (pre-BNZ- [symbol] ) and 7-weeks (with or without BNZ- [symbol] [symbol] treatment) post-huPBMC transplantation. Human CD8+ T-cells highlighted with black arrow.

FIG. 13A depicts the nucleotide and peptide sequence of human CD8 alpha chain.

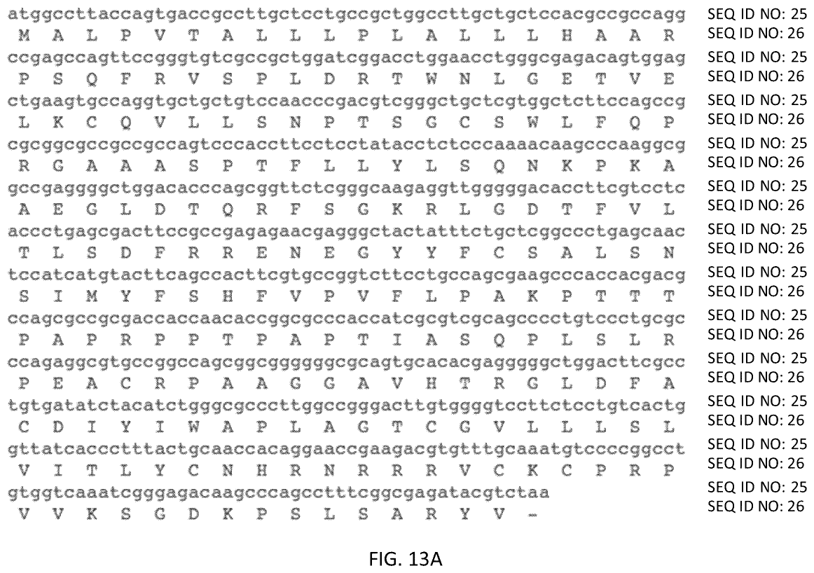

FIG. 13B depicts the nucleotide and peptide sequence of human CD8 beta chain.

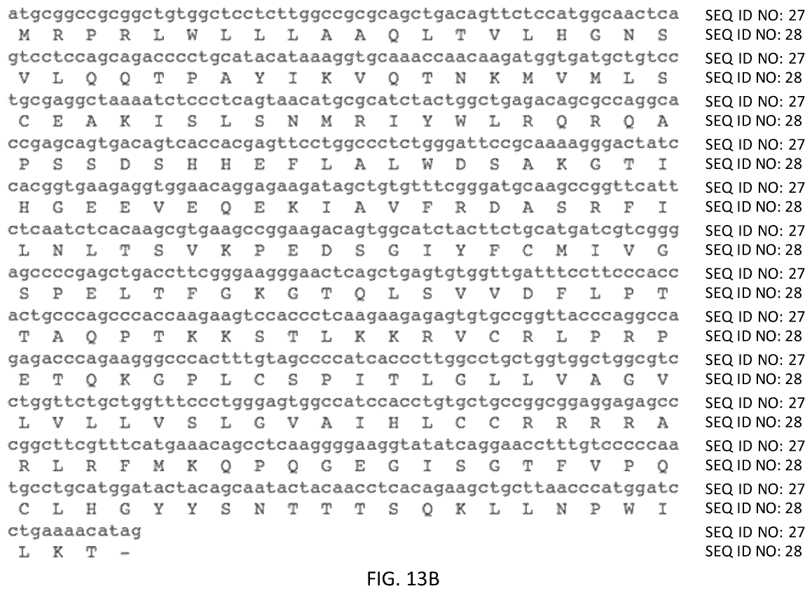

FIG. 14 depicts the nucleotide and peptide sequence of human IL-2.

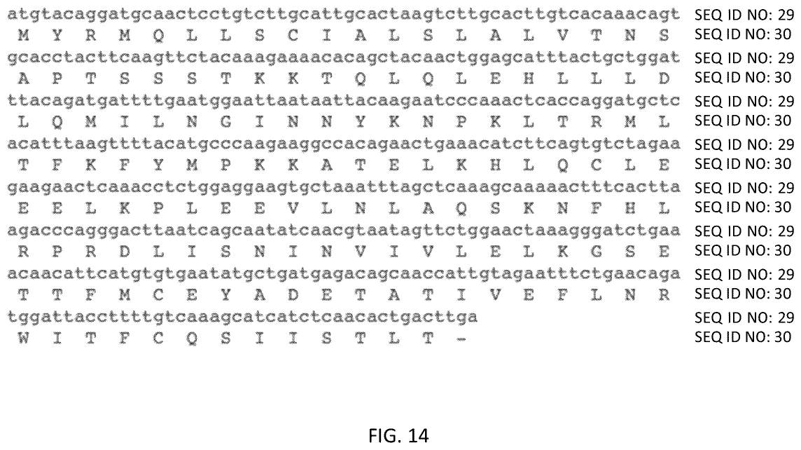

FIG. 15 depicts the nucleotide and peptide sequence of human IL-15.

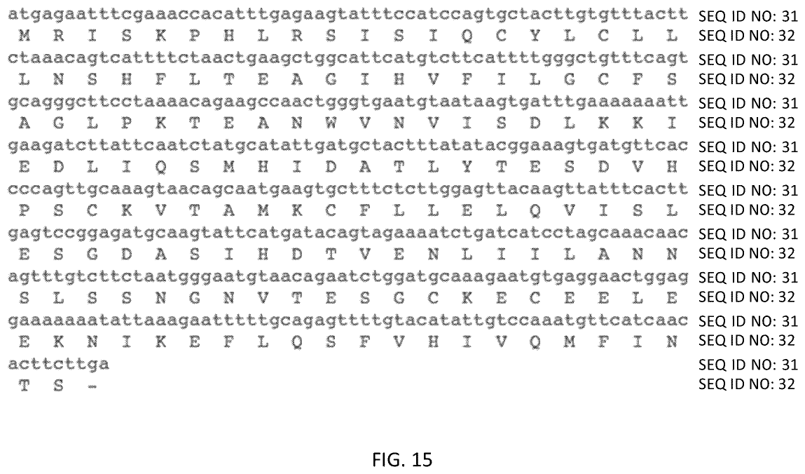

FIG. 16 depicts the nucleotide and peptide sequence of human NKG2A.

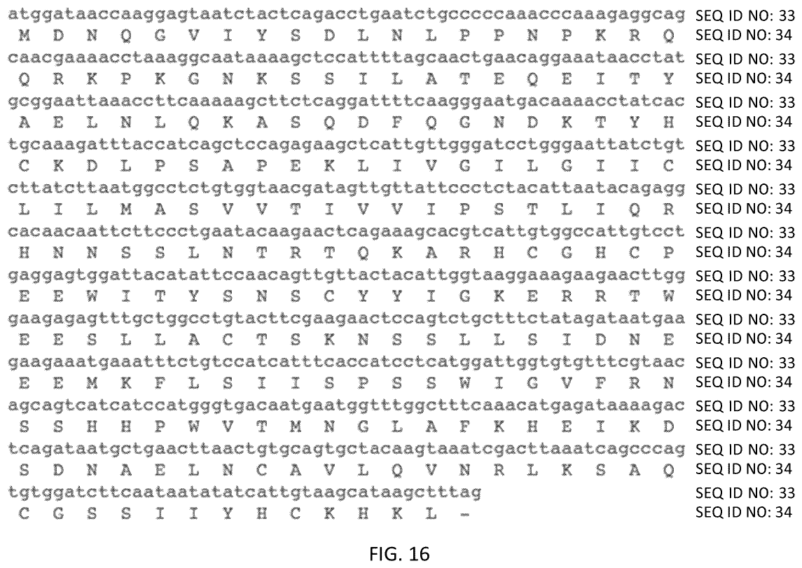

FIG. 17 depicts the nucleotide and peptide sequence of human NKG2B.

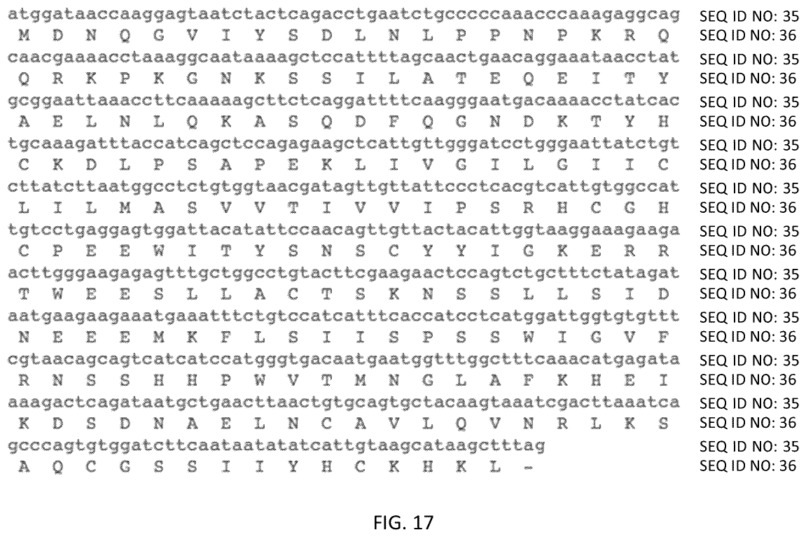

FIG. 18 depicts the nucleotide and peptide sequence of human NKG2C.

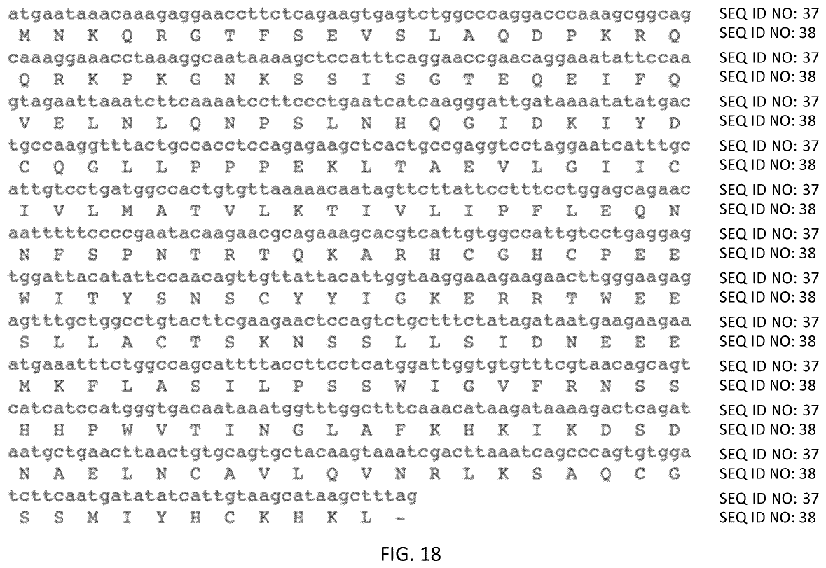

FIG. 19 depicts the nucleotide and peptide sequence of human NKG2D.

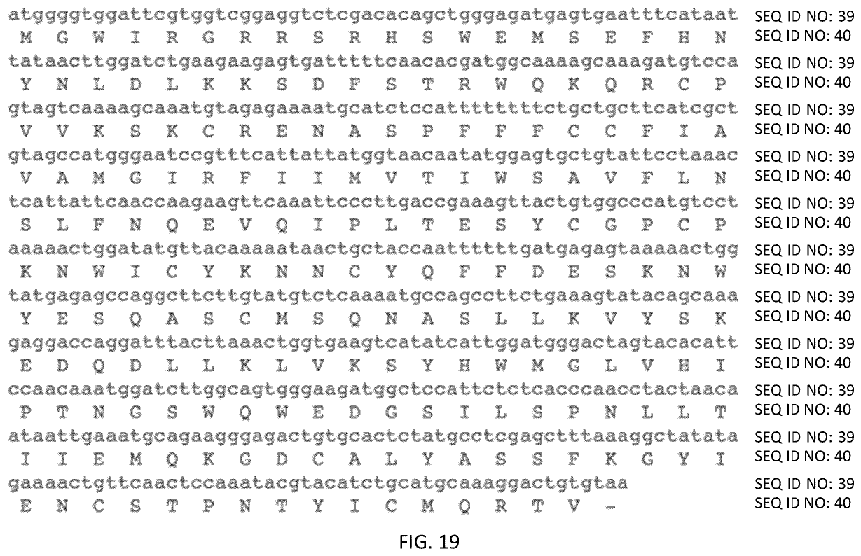

FIG. 20 depicts the nucleotide and peptide sequence of human NKG2E.

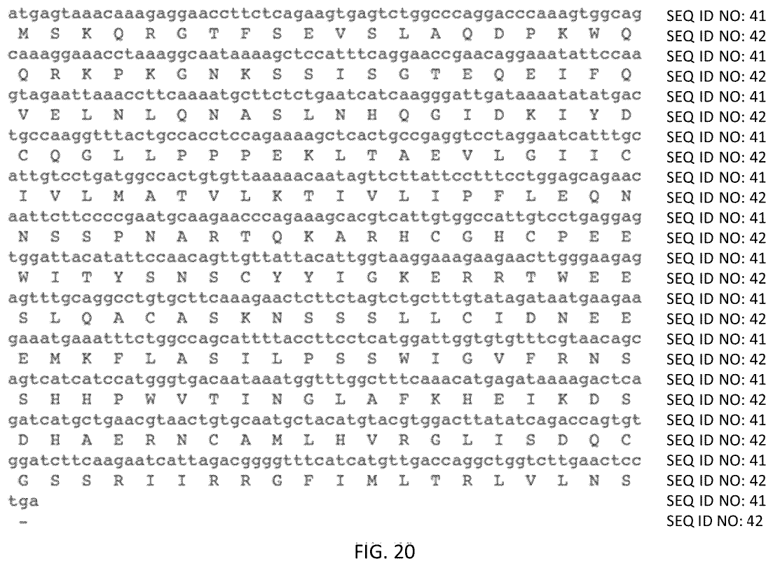

FIG. 21 depicts the nucleotide and peptide sequence of human NKG2F.

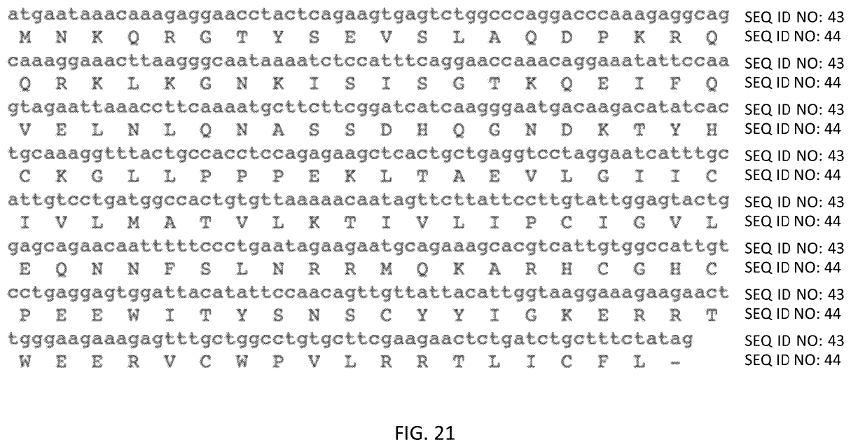

FIG. 22 depicts the nucleotide and peptide sequence of human NKG2H.

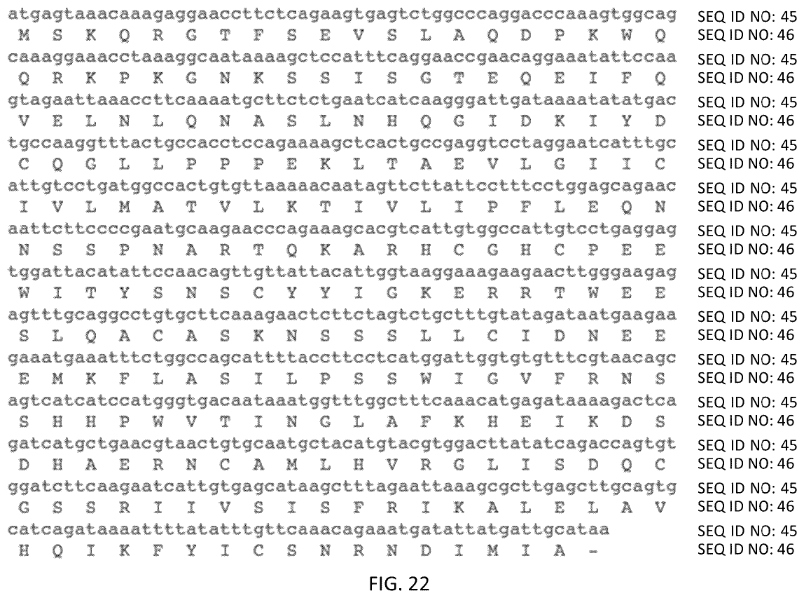

### DETAILED DESCRIPTION

Embodiments herein relate to compositions, methods, and kits comprising one or more therapeutic compounds that modulate signaling by at least one [symbol] c-cytokine family member for inhibiting, ameliorating, reducing a severity of, treating, delaying the onset of, or preventing autoimmune diseases such as alopecia, and alopecia associated disorders. Cytokines of the [symbol] c-family comprise a group of mammalian cytokines that are mainly produced by epithelial, stromal and immune cells and control the normal and pathological activation of a diverse array of lymphocytes. Description of target diseases, as well as methods of administration, production, and commercialization of the therapeutic compounds are disclosed.

### Overview

More than 100 cytokines have been identified so far and are considered to have developed by means of gene duplications from a pool of primordial genes (See Bazan, J. F. 1990, Immunol. Today 11:350-354). In support of this view, it is common for a group of cytokines to share a component in their multi-subunit receptor system. The most well-documented shared cytokine subunit in T cells is the common γ subunit (γc-subunit).

The γc-subunit is shared by 6 known cytokines (Interleukin-2 (IL-2), Interleukin-4 (IL-4), Interleukin-7 (IL-7), Interleukin-9 (IL-9), Interleukin-15 (IL-15), and Interleukin-21 (IL-21), collectively called the “γc-cytokines” or “γc-family cytokines” and plays an indispensable role in transducing cell activation signals for all these cytokines. Additionally, for each of the γc-cytokines, there are one or two private cytokine-specific receptor subunits that when complexed with the γc-subunit, give rise to a fully functional receptor. (See Rochman et al., 2009, Nat Rev Immunol. 9: 480-90.)

The γc-family cytokines are a group of mammalian cytokines that are mainly produced by epithelial, stromal and immune cells and control the normal and pathological activation of a diverse array of lymphocytes. These cytokines are critically required for the early development of T cells in the thymus as well as their homeostasis in the periphery. For example, in the absence of the γc-subunit, T, B and NK cells do not develop in mice. (See Sugamura et al., 1996, Annu. Rev. Immunol. 14:179-205).

The [symbol] c-cytokines are important players in the development of the lymphoid cells that constitute the immune system, particularly T, B, and NK cells. Further, [symbol] c-cytokines have been implicated in various human diseases. Thus, factors that inhibit [symbol] c-cytokine activity would provide useful tools to elucidate the developmental mechanism of subsets of lymphocytes and to treat immune disorders and [symbol] c-cytokine-mediated diseases.

Germ line depletion of the genes encoding the [symbol] c-subunit in mice or mutations of [symbol] c-subunit in humans are known to cause severe combined immunodeficiency (SCID) by disrupting the normal appearance or function of NK, T, and B cells. The importance of the [symbol] c-subunit in the signal transduction of the [symbol] c-cytokines, IL-2, -4, -7, -9, 15, -21, is indicated in studies demonstrating the lack of response of lymphocytes from these mice and human patients to the [symbol] c-cytokines (reviewed in Sugamura et al., 1995 Adv. Immunol. 59:225-277). This indicates that disruption of the interaction between the [symbol] c-subunit and a [symbol] c-cytokine would efficiently block the intracellular signaling events by the [symbol] c-cytokine family members. Therefore, antagonist peptides according to the present embodiments are expected to effectively block the pathogenic changes in humans suffering from the diseases mediated by misregulation of the [symbol] c-cytokine family members.

Applicants present novel compositions, methods, and kits comprising one or more therapeutic compounds that modulate signaling by at least one [symbol] c-cytokine family member for inhibiting, ameliorating, reducing a severity of, treating, delaying the onset of, or preventing autoimmune diseases such as alopecia, and alopecia associated disorders. Applicants have also devised novel, low molecular weight therapeutic compounds herein referred to as “Simul-Block”, which suppress the activity of multiple [symbol] c-cytokines. These low molecular weight therapeutic compounds, which include both chemicals and peptides, are often less immunogenic than antibodies, and can be used as a stand-alone approach, or complementary to antibody-mediated approaches, for modulating [symbol] c-cytokine activity in clinical interventions.

### Pathologies Associated with the γC-Cytokines

Recent studies have indicated that dysregulation of expression and dysfunction of the γc-cytokines could lead to a wide variety of human immunologic and hematopoietic diseases.

### IL-2

While IL-2 was historically considered a prototype T cell growth factor, the generation of a knockout mouse lacking IL-2 expression revealed that IL-2 is not critical for the growth or developmental of conventional T cells in vivo. Over-expression of IL-2, however, leads to a preferential expansion of a subset of T-cells; the regulatory T cells (T-regs). (See Antony et al., 2006, J. Immunol. 176:5255-66.) T-regs suppress the immune responses of other cells and thus act to maintain peripheral tolerance (reviewed in Sakaguchi et al., 2008, Cell 133:775-87). Breakdown of peripheral tolerance is thought to cause autoimmune diseases in humans.

Thus, the immunosuppressive function of T-regs is thought to prevent the development of autoimmune diseases (See Sakaguchi et al., 2008, Cell 133:775-87). T-regs have also been implicated in cancer, where solid tumors and hematologic malignancies have been associated with elevated numbers of T-regs (See De Rezende et al., 2010, Arch. Immunol. Ther. Exp. 58:179-190).

### IL-4

IL-4 is a non-redundant cytokine involved in the differentiation of T helper cells into the Th2 (T-helper type 2) subset, which promotes the differentiation of premature B cells into IgE producing plasma cells. IgE levels are elevated in allergic asthma. Thus, IL-4 is implicated in the development of allergic Asthma. Antibodies targeting IL-4 can be used to treat or even prevent the onset of allergic asthma. (See Le Buanec et al., 2007, Vaccine 25:7206-16.)

### IL-7

IL-7 is essential for B cell development and the early development of T cells in the thymus. In mice, the abnormal expression of IL-7 causes T-cell-associated leukemia. (See Fisher et al., 1993, Leukemia 2:S66-68.) However, in humans, misregulation of IL-7 does not appear to cause T-cell-associated leukemia. In humans, up-regulation of IL-7 either alone or in combination with another [symbol] c-cytokine family member, IL-15, has been implicated in Large Granular Lymphocyte (LGL) leukemia.

### IL-9

The role of IL-9 is still rather uncharacterized compared to other [symbol] c-cytokine family members. Mice depleted of the IL-9 gene appear normal and do not lack any subsets of cells in the lymphoid and hematopoietic compartments. Recent studies, however, reveal an in vivo role for IL-9 in the generation of Th17 (T-helper induced by interleukin-17) cells (See Littman et al., 2010, Cell 140(6):845-58; and Nowak et al., 2009, J. Exp. Med. 206: 1653-60).

### IL-15

IL-15 is critically involved in the development of NK cells, NK-T cells, some subsets of intraepithelial lymphocytes (IELs), [symbol] [symbol] -T cells, and memory-phenotype CD8 T-cells (See Waldmann, 2007, J. Clin. Immunol. 27:1-18; and Tagaya et al., 1996, EMBO J. 15:4928-39.) Over-expression of IL-15 in mice leads to the development of NK-T cell and CD8 cell type T cell leukemia (See Fehniger et al., 2001, J. Exp. Med. 193:219-31; Sato et al. 2011 Blood in press). These experimentally induced leukemias appear similar to LGL (large-granular lymphocyte) leukemia in humans, since in both instances the leukemic cells express CD8 antigen.

It is also suspected that IL-15-mediated autocrine mechanisms may be involved in the leukemic transformation of CD4 T lymphocytes. (See Azimi et al., 1998, Proc. Natl. Acad. Sci. 95:2452-7; Azimi et al., 1999, J. Immunol. 163:4064-72; Azimi et al., 2000, AIDS Res. Hum. Retroviruses 16:1717-22; and Azimi et al., 2001, Proc. Natl. Acad. Sci. 98:14559-64). For example, CD4-tropic HTLV-I, which causes Adult T cell leukemia in humans, induces autocrine growth of virus-transformed T cells through the production of IL-15 and IL-15R [symbol] (Azimi et al., 1998, Proc. Natl. Acad. Sci. 95:2452-7).

In addition to leukemic transformation, recent studies implicate IL-15 in the pathological development of Celiac disease (CD), an autoimmune disease. IL-15 is known to stimulate the differentiation of NK, CD8 and intestinal intraepithelial lymphocyte (IEL) cells into lymphokine-activated killer (LAK) cells by inducing the expression of cytolytic enzymes (i.e., Granzyme and Perforin) as well as interferon- [symbol] . Celiac Disease (denoted CD from herein) is an immune-mediated enteropathy that is triggered by the consumption of gluten-containing food in individuals that express specific HLA-DQ alleles.

The prevalence of this disease is 1% in the western population. The only current treatment for CD is the complete elimination of gluten from the patient's diet. The pathology of CD is mainly caused by extensive damage to the intestinal mucosa, which is caused by activated CD8 T cells that have infiltrated to the intestinal lamina propria. These CD8 T cells appear to be activated through mechanisms involving IL-15. One recent publication demonstrated in mice that ectopic over-expression of IL-15 by enterocytes leads to the development of enteropathy, which closely resembles the lesions in CD patients. Neutralization of IL-15 activity dramatically diminished the pathological changes. Thus, an intervention blocking the activation of CD8 T cells by IL-15 appears to provide an alternative strategy in managing CD to the conventional gluten-free diet.

### IL-21

IL-21 is the most recently discovered member of the [symbol] c-family. Unlike other family members, IL-21 does not appear to have potent growth-promoting effects. Instead, IL-21 is thought to function more as a differentiation factor than a factor controlling cellular proliferation (See Tagaya, 2010, J. Leuk. Biol. 87:13-15).

### Current Strategies for Treating γC-Cytokine-Mediated Disorders

Because the γc-cytokines are thought to be involved in numerous human diseases, several methods of treating γc-cytokine-implicated diseases by inhibiting γc-cytokine family activities have been proposed. These methods include the use of cytokine-specific monoclonal antibodies to neutralize the targeted cytokine's activity in vivo; use of monoclonal antibodies targeting the private cytokine-specific receptor subunits (subunits other than the shared γc-subunit) to selectively inhibit cytokine activity; and use of chemical inhibitors that block the downstream intracellular cytokine signal transduction pathway.

While cytokine-specific antibodies are often the first choice in designing therapeutics, cytokines that share receptor components display overlapping functions (See Paul, W. E., 1989, Cell 57:521-24) and more than one cytokine can co-operate to cause a disease (See Examples described herein). Thus, antibody approaches involving neutralization of a single cytokine may not always be optimal in the treatment of cytokine-implicated human diseases. Alternative therapeutic strategies may involve the use of more than one antibody, where each target a specific cytokine implicated in disease pathogenesis, and/or targeting a specific protein receptor implicated in discase pathogenesis whose activity and/or abundance is directly modulated by γc -cytokine signaling.

Strategies for designing therapeutics that inhibit the function of multiple cytokines via antibodies which recognize a shared receptor component have also been proposed. However, the multi-subunit nature of cytokine receptor systems and the fact that functional receptors for a single cytokine can assume different configurations makes this approach difficult.

For example, a functional IL-15 receptor can be either IL-15Rβ/γc or IL-15Rα/β/γc. (See Dubois et al., 2002, Immunity 17:537-47.) An antibody against the IL-15Rβ receptor (TMβ1), is an efficient inhibitor of the IL-15 function, but only when the IL-15Rα molecule is absent from the receptor complex. (See Tanaka et al., 1991, J. Immunol. 147:2222-28.) Thus, the effectiveness of a monoclonal anti-receptor antibody, whether raised against a shared or a private subunit, can be context-dependent and is unpredictable in vivo.

The polypeptides of the therapeutic compounds, their fragments or other derivatives, or analogs thereof, or cells expressing them can be used as an immunogen to produce antibodies thereto. The term “immunogen” or “epitope”, as used herein, refers to portions of a polypeptide having antigenic or immunogenic activity in an animal, preferably a mammal. In a preferred embodiment, the therapeutic compounds of the present invention encompass a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. The term “antigenic epitope,” as used herein, is defined as a portion of a protein to which an antibody can immune-specifically bind its antigen as determined by any method well known in the art, for example, by immunoassays (Cox et al. 2004 “Immunoassay methods”, in Assay Guidance Manual [internet]). Immuno-specific binding excludes non-specific binding but does not necessarily exclude cross-reactivity with other antigens. Antigenic epitopes need not necessarily be immunogenic. Either the full-length polypeptide or an antigenic peptide fragment of the therapeutic compounds in the present disclosure can be used.

Epitope-bearing polypeptide regions of the therapeutic compounds in the present disclosure can be determined by any method known in the art, for example, by multiple software programs freely available for use, including but not limited to: BepiPred-2.0 (Jespersen et al. 2017 Nucleic Acids Res, 45:W24-W29), SVMTriP (Yao et al. 2012 PLOS One, 7:e45152), and ABCpred (Saha et al. 2006 Proteins, 65:40-8). Antibodies are preferably prepared from these regions or from discrete fragments in these regions. However, antibodies can be prepared from any region of the peptide as described herein. Antibodies may also be developed against specific functional sites, such as the site of ligand binding or sites that are glycosylated, phosphorylated, myristoylated, or amidated. Peptide fragments which function as epitopes may be produced by any conventional means, such as biological production using recombinant technology or chemically through manual or automated peptide synthesis technologies.

Various procedures known in the art may be used for the production of such antibodies and fragments. Epitope-bearing polypeptides of the present invention may be used to induce antibodies according to methods well known in the art including, but not limited to, in vivo immunization, in vitro immunization (Tomimatsu et al. 2014 Methods Mol Biol, 1060:297-307), and phage display methods (Hammers et al. 2014 J Invest Dermatol., 134:e17). If in vivo immunization is used, animals may be immunized with free peptide; however, anti-peptide antibody titer may be boosted by coupling the peptide to a macromolecular carrier, such as keyhole limpet hemacyanin (KLH) or tetanus toxoid. For instance, peptides containing cysteine residues may be coupled to a carrier using a linker such as maleimidobenzoyl-N-hydroxysuccinimide ester (MBS), while other peptides may be coupled to carriers using a more general linking agent such as glutaraldehyde. Antibodies gencrated against the polypeptides corresponding to each of the therapeutic compounds of the present invention can be obtained by direct injection of the polypeptides into an animal or by administering the polypeptides to an animal, preferably a nonhuman. Animals such as rabbits, rats, mice, and goats can be immunized with either free or carrier-coupled peptides, or artificially branched forms known as multiple antigenic peptides (MAPs), for instance, by intraperitoneal and/or intradermal injection of emulsions containing about 100 ug of peptide or carrier protein and Freund's adjuvant or any other adjuvant known for stimulating an immune response. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

For preparation of monoclonal antibodies, any technique which provides antibodies produced by continuous cell line cultures can be used. Examples include the hybridoma technique (Kohler et al. 1975 Nature, 256:495-7), the trioma technique, the human B-cell hybridoma technique (Kozbor et al. 1983 Immunology Today, 4:72-9), and the EBV-hybridoma technique to produce human monoclonal antibodies (Kozbor et al. 1982 Proc Natl Acad Sci, 79:6651-55). Techniques described for the production of single chain antibody fragments (scFv) (Blažek et al. 2003 Folia Microbiol, 48:687-98) can be adapted to produce single chain antibodies to immunogenic polypeptides derived from the therapeutic compounds in the present invention.

Humanized antibodies are antibody molecules derived from a non-human species antibody that binds the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (Riechmann et al. 1988 Nature 332:323-7). Antibodies can be humanized using a variety of techniques known in the art including, for example, CDR-grafting (Williams et al. 2010 “Humanising Antibodies by CDR Grafting”, in Antibody Engineering), vencering or resurfacing (Padlan 1991 Mol Immunol 28:489-98; Studnicka et al. 1994 Protein Eng 7:805-14; Roguska et al. 1994 Proc Natl Acad Sci 91:969-73), and chain shuffling (Guo-Qiang et al. 2009 Methods Mol Biol 562:133-42).

Completely human antibodies are particularly desirable for therapeutic treatment of human patients. Human antibodies can be made by a variety of methods known in the art including phage display using antibody libraries derived from human immunoglobulin sequences (Frenzel, et al. 2017 Transfus Med Hemother 44:312-18, Vaughan, et al. 1996 Nature 14:309-14). Human antibodies which recognize a selected epitope can also be generated using a technique referred to as “guided selection.” In this approach, a selected non-human monoclonal antibody, c.g., a mouse antibody, is used to guide the selection of a completely human antibody recognizing the same epitopc. (Jespers et al. 1994 Biotechnology 12:899-903).

Also, transgenic mice may be used to express human antibodies to immunogenic polypeptides derived from the therapeutic compounds in the present invention (Laffleur et al. 2012 Methods Mol Biol, 901:149-59). Transgenic mice, which are incapable of expressing functional endogenous immunoglobulins, can be used to express human immunoglobulin genes. For example, the human heavy and light chain immunoglobulin genc complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide corresponding to a therapeutic compound of the present invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg et al. 1995 Int Rev Immunol. 13:65-93.

Antibodies of the present invention include, but are not limited to, polyclonal, monoclonal, multi-specific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immune-specifically binds an antigen. The immunoglobulin molecules of the invention can be of any type (e.g., IgG, IgE, IgM, Ig|D, IgA and IgY), class (c.g., IgG1, IgG2, lgG3, IgG4, IgA1 and IgA2) or subclass of immunoglobulin molecule. In a preferred embodiment, the immunoglobulin is an IgGI isotype. In another preferred embodiment, the immunoglobulin is an IgG2 isotype. In another preferred embodiment, the immunoglobulin is an IgG4 isotype. Immunoglobulins may have both a heavy and light chain. An array of IgG, IgE, IgM, IgD, IgA, and IgY heavy chains may be paired with a light chain of the kappa or lambda forms.

### Targeting JAK3, as an Existing Alternative Example for the Inhibition of Multiple c-cytokines

The interaction between the [symbol] c-subunit and a [symbol] c-cytokine leads to the activation of an intracellular protein tyrosine kinase called Janus kinase 3 (Jak3). Jak3, in turn, phosphorylates multiple signaling molecules including STAT5, and PI3 kinase. The interaction of the [symbol] c-subunit and Jak3 is very specific. In fact, there is no other receptor molecule that recruits Jak3 for signal transduction. (See O'Shea, 2004, Ann. Rheum. Dis. 63:(suppl. II):ii67-7.) Thus, the inhibition of cytokine signaling through the [symbol] c-subunit can be accomplished by blocking the activity of Jak3 kinase. Accordingly, multiple small molecule chemical inhibitors that target the kinase activity of Jak3 have been introduced to the market. (See Pesu et al., 2008, Immunol. Rev. 223:132-142.) One such example is CP690,550.

The major shortcoming of these protein kinase inhibitors is the lack of specificity to Jak3 kinase. These drugs intercept the binding of ATP (adenosine-triphosphate) molecules to Jak3 kinase, a common biochemical reaction for many protein kinases, and thus tend to block the action of multiple intracellular protein kinases that are unrelated to Jak3 kinase whose actions are critically needed for the well-being of normal cells in various tissues. Thus, more specific inhibitors of signaling through the [symbol] c-subunit are needed.

There is therefore a great need for an alternative non-small molecule chemical strategy for treating γc-cytokine-implicated diseases.

### Discovery of the C-Box

The C-terminus (the D-helix) of the [symbol] c-cytokines contains the proposed site for interacting with the common [symbol] c-subunit of the multi-unit cytokine receptors. (Bernard et al., 2004 J. Biol. Chem. 279:24313-21.) Comparison of the biochemical properties of the amino acids of all [symbol] c-cytokines identified in mice and humans revealed that the chemical nature of the amino acids, for example, hydrophobicity, hydrophilicity, base/acidic nature, are conserved, if not identical, at many positions in the D-helix across the members of the [symbol] c-cytokine family.

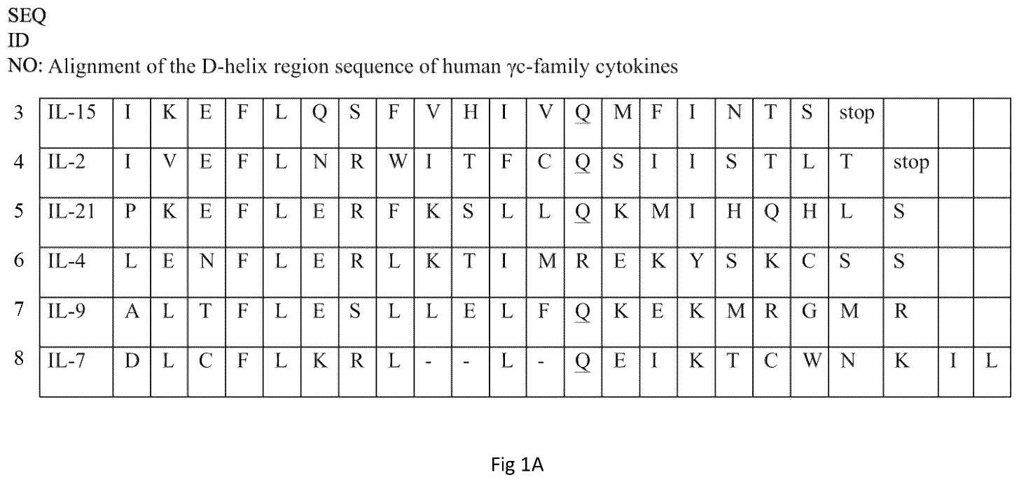

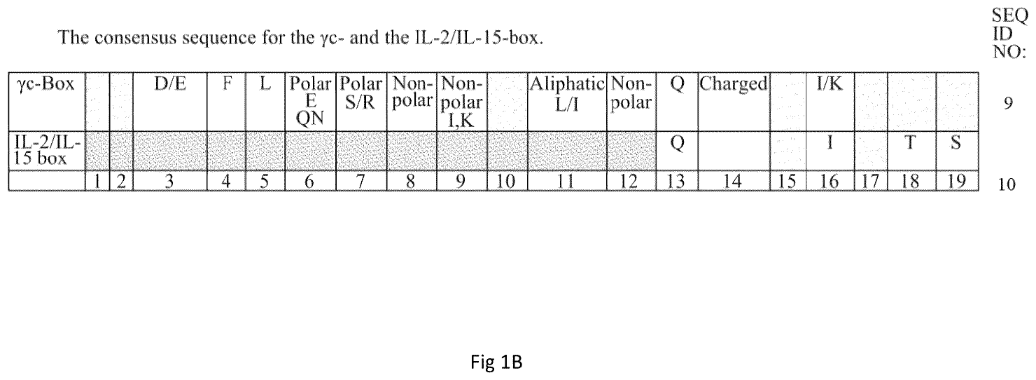

In contrast, the sequence of IL-13, which is related to the [symbol] c-cytokine, IL-4, but does not bind to the [symbol] c-subunit, does not exhibit significant homology in the D-helix region to the [symbol] c-cytokines, suggesting that the sequence homology in the D-helix region is correlated with binding to the [symbol] c-subunit. As shown in FIG. 1A, alignment of the amino acid sequences of the D-helix region of [symbol] c-cytokine family members in humans reveals a motif of moderate sequence homology in these cytokines referred to herein as “the [symbol] c-box”.

The [symbol] c-box (SEQ ID NO: 9) comprises 19 amino acids where out of the 19 positions, positions 4, 5, and 13 are fully conserved as Phenylalanine, Leucine, and Glutamine, respectively. Less conservation is observed at positions 6, 7 and 11 of the [symbol] c-box where the amino acid is one of two or three related amino acids that share physico-chemical properties: position 6 may be occupied by the polar amino acids Glutamate, Asparagine or Glutamine; non-polar amino acids Serine or Arginine can occupy position 7; and position 11 is occupied by either of the non-polar aliphatic amino acids Leucine or Isoleucine. Positions 9 and 16 may be occupied by the either the non-polar amino acid Isoleucine or the polar amino acid Lysine. See FIG. 1B. Some differences in the amino acid composition of the [symbol] c-box are observed at positions 9 and 16 amongst subfamilies of the [symbol] c-cytokines. Comparison of the [symbol] c-cytokines across species indicates that Isoleucine is often present at the 9 and 16 positions in the IL-2/15 subfamily, whereas the other [symbol] c-family members often possess Lysine in these positions. Not wishing to be bound by a particular theory, Isoleucine and Lysine are biochemically different and thus may impart specific conformational differences between the IL-2/15 subfamily and other [symbol] c-cytokines.

Conservation of the [symbol] c-box motif between [symbol] c-cytokines is supported by findings that a Glutamine (Gln, Q) residue located in the D-helix region is critical for the binding of the [symbol] c-cytokines to the [symbol] c-subunit. (Bernard et al., 2004 J. Biol. Chem. 279: 24313-21.)

### Modulators of c-Cytokine Activity

The activity of [symbol] c-family cytokines may be blocked by disrupting the interaction between the [symbol] c-cytokine and the [symbol] c-subunit, for example by introducing a competitive inhibitor which can interact with the [symbol] c-subunit without stimulating signaling through the multi-subunit cytokine receptors. Not to be bound by a particular theory, the conserved [symbol] c-box motif, which participates in binding of the [symbol] c-family cytokines to the [symbol] [symbol] c-subunit, presents a core base amino acid sequence which can be utilized to design peptide modulators of [symbol] c-cytokine signaling.

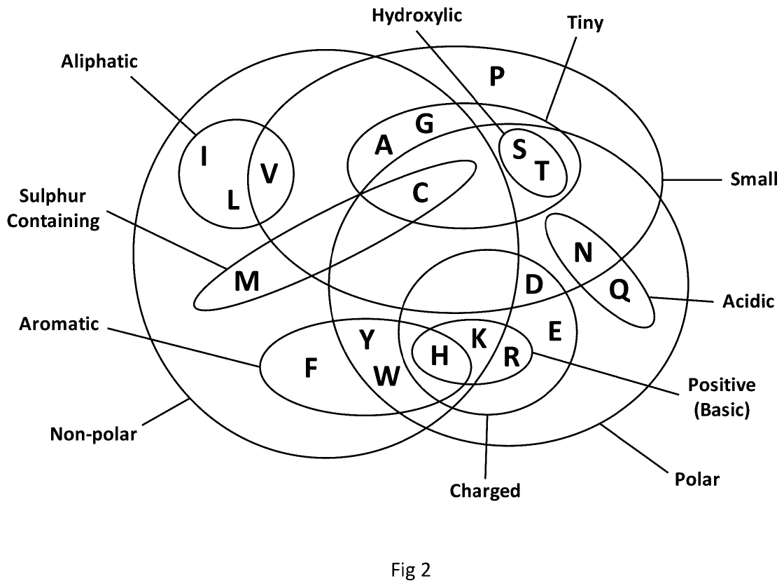

The core [symbol] c-box amino acid sequence comprises: D/E-F-L-E/Q/N-S/R-X-I/K-X-L/I-X-Q (SEQ ID NO: 2) (where X denotes any amino acid). Embodiments described herein relate to custom peptide derivatives of the core [symbol] c-box amino acid sequence which can modulate the activity of one or more [symbol] c-cytokines. Custom peptide derivatives include any peptide whose partial amino acid sequence shows approximately 50%, 50-60%, 60-70%, 70-80%, 80%, 90%, 95%, 97%, 98%, 99% or 99.8% identity to the core [symbol] c-box amino acid sequence. Custom peptide derivatives further include any peptide wherein a partial amino acid sequence of that peptide derivative comprises amino acids with similar physico-chemical properties to the amino acids of the core [symbol] c-box. For example, amino acids with similar physico-chemical properties would include Phenylalanine, Tyrosine, Tryptophan, and Histidine, which are aromatic amino acids. FIG. 2 shows a diagrammed representation of amino acids with similar physico-chemical properties which may be may be substituted for the amino acids comprising the core [symbol] c-box. Peptide derivatives of the core [symbol] c-box may be 11, 12, 13, 14, 15, 16, 17, 18, 19, 20, 21, 22, 23, 24, 25-30, 30-35, 35-40, 40-45, 45-50, or more than 50 amino acids in length. In some embodiments, the custom peptide derivatives may be conjugated to the N-termini, C-termini and/or to the side residues of existing biological proteins/peptides.

Based on the identification of the conserved [symbol] c-box motif in cytokines which bind to the [symbol] c-subunit, Applicants have devised a novel, 19-mer custom derivative peptide which is an artificial composite peptide combining the amino acid sequence of the human IL-2 and IL-15 [symbol] c-box. The 19-mer peptide, herein referred to as BNZ- [symbol] [symbol] [symbol] consists of the amino acid sequence: [symbol] -K-E- [symbol] - [symbol] -Q-R-F-I-H- [symbol] -V- [symbol] -S-I- [symbol] -N- [symbol] - [symbol] (SEQ ID NO: 1), where the amino acids depicted by bold characters are conserved between IL-2 and IL-15 and the underlined amino acids represent positions where the physico-chemical properties of the amino acids are conserved.

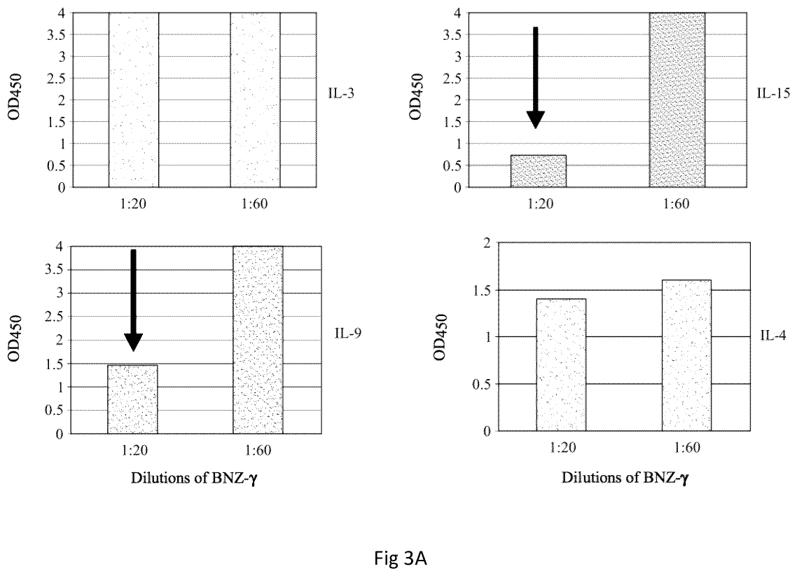

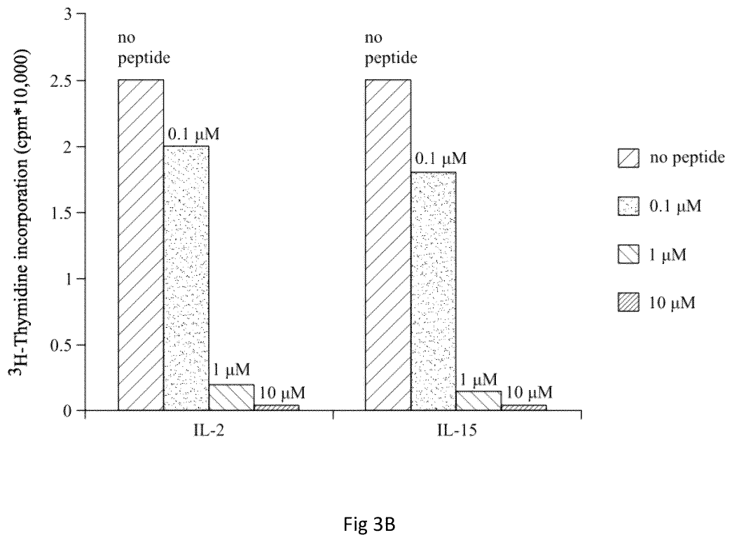

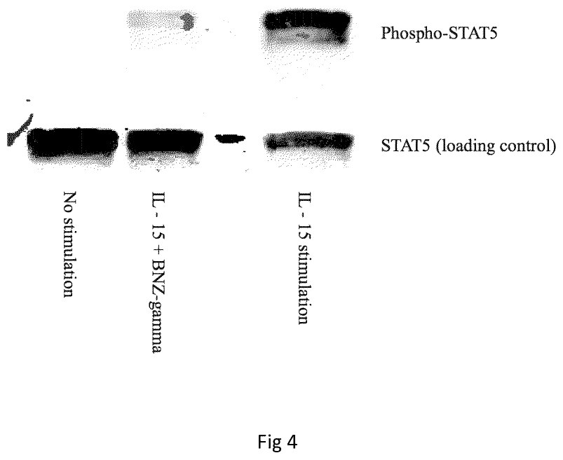

Applicants discovered that the 19-mer BNZ- [symbol] [symbol] [symbol] suppresses IL-15 and IL-9 induced cellular proliferation, but not IL-3 or IL-4 induced cellular proliferation. See FIG. 3A and EXAMPLE 2. Applicants further demonstrated that BNZ- [symbol] [symbol] inhibits IL-15 mediated phosphorylation of the intracellular cytokine signal transduction molecule, STAT-5. See FIG. 4 and EXAMPLE 5. These results demonstrate that custom peptide derivatives of the conserved [symbol] c-box motif can modulate the activity of multiple [symbol] c-cytokines.

Several embodiments relate to one or more therapeutic compounds that modulate signaling by at least one [symbol] c-cytokine family member for inhibiting, ameliorating, reducing a severity of, treating, delaying the onset of, or preventing autoimmune diseases such as alopecia, and alopecia associated disorders. In some embodiments, the therapeutic compound is one or more of a [symbol] c-cytokine antagonist peptide, a [symbol] c-cytokine antagonist peptide derivative, anti-CD8 antibody, anti-IL-2 antibody, anti-IL-15 antibody, anti-NKG2A antibody, or a combination thereof.

In some embodiments, [symbol] c-cytokine antagonist peptides and derivatives thereof, which are also referred to herein as custom derivative peptides or composite peptide derivatives of the 19-mer BNZ- [symbol] [symbol] amino acid sequence, I-K-E-F-L-Q-R-F-I-H-I-V-Q-S-I-I-N-T-S (SEQ ID NO: 1), can inhibit the activity of one or more [symbol] c-cytokines. Custom peptide derivatives of the 19-mer BNZ- [symbol] [symbol] amino acid sequence include any peptide whose partial amino acid sequence shows approximately 50%, 50-60%, 60-70%, 70-80%, 80%, 90%, 95%, 97%, 98%, 99% or 99.8% identity to amino acid sequence: I-K-E-F-L-Q-R-F-I-H-I-V-Q-S-I-I-N-T-S (SEQ ID NO: 1). Custom peptide derivatives further include any peptide wherein a partial amino acid sequence of that peptide derivative comprises amino acids with similar physico-chemical properties to the amino acids of sequence: I-K-E-F-L-Q-R-F-I-H-I-V-Q-S-I-I-N-T-S (SEQ ID NO: 1).

In several embodiments, the amino acid residues of the custom derivative peptides retain similar physico-chemical properties with the amino acid residues of BNZ- [symbol] [symbol] [symbol] but exhibit different biological inhibition specificity to the 6 [symbol] c-cytokine family members from that of the original 19-mer peptide. Peptide derivatives of BNZ-may be 19, 20, 21, 22, 23, 24, 25-30, 30-35, 35-40, 40-45, 45-50, or more than 50 amino acids in length.

In some embodiments, the custom peptide derivatives may be conjugated to the N-termini, C-termini and/or to the side residues of existing biological proteins/peptides. In some embodiments, peptide derivatives of BNZ- [symbol] [symbol] may be conjugated to other moieties through the N-terminus, C-terminus, or side chains of the composite peptide. The other moieties may include proteins or peptides that stabilize the composite peptide, or other moieties, including without limitation, bovine serum albumin (BSA), albumin, Keyhole Limpet Hemocyanin (KLH), Fc region of IgG, a biological protein that functions as scaffold, an antibody against a cell-specific antigen, a receptor, a ligand, a metal ion and Poly Ethylene Glycol (PEG).

In some embodiments, any of the custom peptide derivatives disclosed herein can comprise one or more intra-peptide hydrocarbon linker elements. In some embodiments, the 19-mer BNZ- [symbol] (SEQ ID NO: 1) comprises one or more intra-peptide hydrocarbon linker elements. In some embodiments, the 19-mer BNZ- [symbol] (SEQ ID NO: 1) comprises one or more intra-peptide hydrocarbon linker elements that connect two separate amino acids positioned 4 residues apart on SEQ ID NO: 1. In some embodiments, the 19-mer BNZ- [symbol] (SEQ ID NO: 1) comprises one or more intra-peptide hydrocarbon linker elements that connect two separate amino acids positioned 7 residues apart on SEQ ID NO: 1. In some embodiments, the 19-mer BNZ-γ (SEQ ID NO: 1) comprises one or more intra-peptide hydrocarbon linker elements that connect two separate amino acids positioned 4 residues apart on SEQ ID NO: 1 and 7 residues apart on SEQ ID NO: 1.

Several embodiments relate to custom derivative peptides of the amino acid sequence, I-K-E-F-L-Q-R-F-I-H-I-V-Q-S-I-I-N-T-S (SEQ ID NO: 1), which can inhibit the activity of one or more [symbol] c-cytokines. Custom peptide derivatives of the amino acid sequence include any peptide whose partial amino acid sequence shows approximately 50%, 50-60%, 60-70%, 70-80%, 80%, 90%, 95%, 97%, 98%, 99% or 99.8% identity to amino acid sequence: I-K-E-F-L-Q-R-F-I-H-I-V-Q-S-I-I-N-T-S (SEQ ID NO: 1). Custom peptide derivatives further include any peptide wherein a partial amino acid sequence of that peptide derivative comprises amino acids with similar physico-chemical properties to the amino acids of sequence: I-K-E-F-L-Q-R-F-I-H-I-V-Q-S-I-I-N-T-S (SEQ ID NO: 1).

In several embodiments, the amino acid residues of the custom derivative peptides retain similar physico-chemical properties with the amino acid residues of SEQ ID NO: 1 [symbol] [symbol] but exhibit different biological inhibition specificity to the 6 [symbol] c-cytokine family members from that of the original 19-mer peptide. Peptide derivatives of SEQ ID NO: 1 [symbol] may be less than 19, 20, 21, 22, 23, 24, 25-30, 30-35, 35-40, 40-45, 45-50, or more than 50 amino acids in length.

In some embodiments, the custom peptide derivatives may be conjugated to the N-termini, C-termini and/or to the side residues of existing biological proteins/peptides. In some embodiments, the composite peptide of SEQ ID NO: 1 may be conjugated to other moieties through the N-terminus, C-terminus, or side chains of the composite peptide. In some embodiments, the other moieties may include proteins or peptides that stabilize the composite peptide, or other moieties, including without limitation, bovine serum albumin (BSA), albumin, Keyhole Limpet Hemocyanin (KLH), Fc region of IgG, a biological protein that functions as scaffold, an antibody against a cell-specific antigen, a receptor, a ligand, a metal ion and Poly Ethylene Glycol (PEG).

In some embodiments, any of the custom peptide derivatives disclosed herein can comprise one or more intra-peptide hydrocarbon linker elements. In some embodiments, the composite peptide of SEQ ID NO: 1 comprises one or more intra-peptide hydrocarbon linker elements. In some embodiments, the composite peptide of SEQ ID NO: 1 comprises one or more intra-peptide hydrocarbon linker elements that connect two separate amino acids positioned 4 residues apart on SEQ ID NO: 1. In some embodiments, the composite peptide of SEQ ID NO: 1 comprises one or more intra-peptide hydrocarbon linker elements that connect two separate amino acids positioned 7 residues apart on SEQ ID NO: 1. In some embodiments, the composite peptide of SEQ ID NO: 1 comprises one or more intra-peptide hydrocarbon linker elements that connect two separate amino acids positioned 4 residues apart on SEQ ID NO: 1 and 7 residues apart on SEQ ID NO: 1.

Several embodiments relate to custom peptide derivatives of the [symbol] c-box motifs of IL-15, IL-2, IL-21, IL-4, IL-9, or IL-7, which are depicted in FIG. 1A. Other embodiments relate to custom derivative peptides which are artificial composite peptides combining the amino acid sequence of two or more of the human IL-15, IL-2, IL-21, IL-4, IL-9, and IL-7 [symbol] c-box motifs. Several embodiments relate to custom peptide derivatives of the of the [symbol] c-box motifs of IL-15, IL-2, IL-21, IL-4, IL-9, or IL-7 having a partial amino acid sequence that shows approximately 50%, 50-60%, 60-70%, 70-80%, 80%, 90%, 95%, 97%, 98%, 99% or 99.8% identity to amino acid sequences of the of the [symbol] c-box motifs of IL-15, IL-2, IL-21, IL-4, IL-9, or IL-7. Custom peptide derivatives of the of the [symbol] c-box motifs of IL-15, IL-2, IL-21, IL-4, IL-9, or IL-7 further include any peptide wherein a partial amino acid sequence of that peptide derivative comprises amino acids with similar physico-chemical properties to the amino acids of sequence of the [symbol] c-box motifs of IL-15, IL-2, IL-21, IL-4, IL-9, or IL-7.

Several embodiments relate to custom peptide derivatives that would inhibit the function of one, all, or selective members of the [symbol] c-cytokines. In some embodiments, the custom peptide derivatives selectively target individual [symbol] c-cytokine family members. For example, a custom peptide derivative can selectively inhibit the function of IL-2, IL-4, IL-7, IL-9, IL-15, or IL-21. In other embodiments, a custom peptide derivative can inhibit 2 or more [symbol] c-cytokine family members.

For example, the custom peptide derivatives of the present embodiments can selectively inhibit the function of IL-2 in combination with one or more of IL-4, IL-7, IL-9, IL-15, and IL-21; IL-4 in combination with one or more of IL-2, IL-7, IL-9, IL-15, and IL-21; IL-7 in combination with one or more of IL-2, IL-4, IL-9, IL-15, and IL-21; IL-9 in combination with one or more of IL-2, IL-4, IL-7, IL-15, and IL-21; IL-15 in combination with one or more of IL-2, IL-4, IL-7, IL-9, and IL-21; or IL-21 in combination with one or more of IL-2, IL-4, IL-7, IL-9, and IL-15. In other embodiments, custom peptide derivatives can comprehensively target all [symbol] c-cytokine family members.

Not wishing to be bound by a particular theory, the custom peptide derivatives can inhibit the function of all or selective members of the [symbol] c-cytokines by diminishing the binding of [symbol] c-cytokines to the [symbol] c-subunit, for example, as a competitive inhibitor. Such custom peptide derivatives may be used in diverse applications, including as a clinical drug.

Several embodiments relate to custom peptide derivatives that would modulate (including enhance or reduce) the function of one, two, or more of selective members of the [symbol] c-cytokines. In some embodiments, the custom peptide derivatives selectively target individual [symbol] c-cytokine family members. For example, a custom peptide derivative can selectively enhance or inhibit the function of IL-2, IL-4, IL-7, IL-9, IL-15, or IL-21. In other embodiments, a custom peptide derivative can enhance or inhibit two or more [symbol] c-cytokine family members.

In some embodiments, one or more of the custom peptide derivatives of the conserved [symbol] c-box motif disclosed herein can inhibit the activity of one or more [symbol] c-cytokines. In some embodiments, one or more of the custom peptide derivatives of the conserved [symbol] c-box motif disclosed herein can inhibit the activity of one or more [symbol] c-cytokines by suppressing cell proliferation induced by the one or more [symbol] c-cytokines. In some embodiments, one or more of the custom peptide derivatives of the conserved [symbol] c-box motif disclosed herein can inhibit the activity of one or more [symbol] c-cytokines by inhibiting phosphorylation of the intracellular cytokine signal transduction molecule mediated by the one or more [symbol] c-cytokines. In some embodiments, one or more of the custom peptide derivatives of the conserved [symbol] c-box motif disclosed herein can inhibit the activity of one or more [symbol] c-cytokines by suppressing cell proliferation induced by the one or more [symbol] c-cytokines and by inhibiting phosphorylation of the intracellular cytokine signal transduction molecule mediated by the one or more [symbol] c-cytokines. In some embodiments, one or more of the custom peptide derivatives of the conserved [symbol] c-box motif disclosed herein can inhibit the activity of one or more [symbol] c-cytokines by one or more other mechanisms.

In some embodiments, one or more of the peptide sequences disclosed herein suppress proliferation of one or more cell types induced by one or more of the cytokines disclosed herein (e.g., IL-2, IL-4, IL-7, IL-9, IL-15, and IL-21). In some embodiments, one or more of the peptide sequences disclosed herein suppress proliferation of one or more cell types induced by all of the cytokines disclosed herein. In some embodiments, one or more of the peptide sequences disclosed herein suppress proliferation of one or more cell types induced by some but not all of the cytokines disclosed herein. In some embodiments, SEQ ID NO: 1 suppresses IL-2 IL-9, and IL-15 induced cellular proliferation.

In some embodiments, one or more of the custom peptide derivatives of the conserved [symbol] c-box motif disclosed herein can inhibit the activity of one or more [symbol] c-cytokines by inhibiting phosphorylation of one or more intracellular cytokine signal transduction molecules mediated by the one or more [symbol] c-cytokines disclosed herein (e.g., IL-2, IL-4, IL-7, IL-9, IL-15, and IL-21). In some embodiments, one or more of the custom peptide derivatives of the conserved [symbol] c-box motif disclosed herein can inhibit phosphorylation of one or more intracellular cytokine signal transduction molecules mediated by all of the [symbol] c-cytokines disclosed herein. In some embodiments, one or more of the custom peptide derivatives of the conserved [symbol] c-box motif disclosed herein can inhibit phosphorylation of one or more intracellular cytokine signal transduction molecules mediated by some but not all of the [symbol] c-cytokines disclosed herein.

Also, for example, the peptides as disclosed herein may be used to inhibit IL-15 mediated phosphorylation of the intracellular cytokine signal transduction molecule STAT-5.

Provided herein are composite peptides, and compositions, methods, and kits to modulate [symbol] c-cytokine signaling. The terms “composite peptide,” “composite peptide derivative,” “custom peptide,” “antagonist peptides,” “antagonist peptides derivatives,” “oligopeptide,” “polypeptide,” “peptide,” and “protein” can be used interchangeably when referring to the “custom peptide derivatives” provided in accordance with the present embodiments and can be used to designate a series of amino acid residues of any length. The peptides of the present embodiments may be linear or cyclic. The peptides of the present embodiments may include natural amino acids, non-natural amino acids, amino acids in the (D) stereochemical configuration, amino acids in the (L) stereochemical configuration, amino acids in the (R) stereochemical configuration, amino acids in the (S) stereochemical configuration, or a combination thereof.

Peptides of the present embodiments may also contain one or more rare amino acids (such as 4-hydroxyproline or hydroxylysine), organic acids or amides and/or derivatives of common amino acids, such as amino acids having the C-terminal carboxylate esterified (e.g., benzyl, methyl or ethyl ester) or amidated and/or having modifications of the N-terminal amino group (e.g., acetylation or alkoxycarbonylamino), with or without any of a wide variety of side chain modifications and/or substitutions. Side chain modifications, substitutions or a combination thereof that may be present in the custom peptide derivatives of the present embodiments include, but are not limited to, α-methyl, α-alkenyl, alkylation, methylation, benzylation, t-butylation, tosylation, alkoxycarbonylamino, and the like.

Residues other than common amino acids that may be present include, but are not limited to, penicillamine, tetramethylene cysteine, pentamethylene cysteine, mercaptopropionic acid, norleucine, pentamethylene-mercaptopropionic acid, 2-mercaptobenzene, 2-mercaptoaniline, 2-mercaptoproline, ornithine, aminoisobutyric acid, diaminobutyric acid, aminoadipic acid, m-aminomethylbenzoic acid, and diaminopropionic acid.

Peptides of the present embodiments can be produced and obtained by various methods known to those skilled in the art. For example, the peptide may be produced by genetic engineering, based on the nucleotide sequence coding for the peptide of the present embodiments, or chemically synthesized by means of peptide solid-phase synthesis and the like, or produced and obtained in their combination. One skilled in the art of solid-phase peptide synthesis can readily incorporate natural or non-natural amino acids in the (D) as well as (L), or the (R) as well as (S), stereochemical configuration. It will also be apparent to one skilled in the art of solid-phase peptide synthesis to produce and obtain peptides containing one or more intra-peptide hydrocarbon linker elements of the present embodiments utilizing α-substituted (such as α-alkenyl) natural or non-natural amino acids in one or more of (D), (L), (R) or (S), stereochemical configurations, or a combination thereof. In some embodiments, an intra-peptide hydrocarbon linker element linking α-substituted amino acids (e.g., α-alkenyl amino acids) can be generated by catalyzing one or more ring-closing metathesis. In some embodiments, one or more intra-peptide hydrocarbon linker elements can be generated by catalyzing a ring-closing metathesis using benzylidenebis(tricyclohexyl-phosphine)-dichlororuthenium (Grubb's catalyst) on the resin-bound peptide during peptide synthesis. In some embodiments, other ring-closing synthesis reactions and/or mechanisms during one or more known peptide synthesis processes are also contemplated. One skilled in the art can synthesize the custom peptide derivatives based on the present disclosure of the conserved [symbol] c-box motif and knowledge of the biochemical properties of amino acids as described in FIG. 2.

Peptides of the present embodiments may also comprise two or more α-alkenyl substituted amino acids. In some embodiments, the two or more α-alkenyl substituted amino acids are linked via one or more intra-peptide hydrocarbon linker elements incorporated at the α-alkenyl substituted amino acids. In some embodiments, the α-alkenyl substituted amino acids are utilized to catalyze the formation of an intra-peptide hydrocarbon linker element by ring-closing metathesis during peptide synthesis. Intra-peptide linker elements join separate amino acids on the same sequence of a custom peptide derivative of the present disclosure. In some embodiments, the peptides of the present disclosure are linear or cyclic.

In some embodiments, one or more intra-peptide hydrocarbon linker elements are incorporated at amino acid positions that correlate with a single α-helical turn in a secondary structure of the composite peptide. In some embodiments, when the composite peptide comprises one or more non-contiguous single α-helical turns, the amino acid positions that correlate with a single α-helical turn of the composite peptide correspond to amino acid positions i and i+4 of the composite peptide, where i is the first amino acid position of the single α-helical turn and i+4 is the last amino acid position of the single α-helical turn, and wherein amino acid positions i and i+4 comprise alpha-alkenyl substituted amino acids, and where i and i+4 are positioned 4 residues apart (4 spaced).

In some embodiments, one skilled in the art of solid-phase peptide synthesis can readily synthesize composite peptides comprising more than one intra-peptide hydrocarbon linker elements such that the composite peptide comprises more than one single α-helical turn. In some embodiments, the more than one single α-helical turns are non-contiguous, i.e., the more than one single α-helical turns do not share a substituted amino acid. For example, in some embodiments, the composite peptide can comprise one or more intra-peptide hydrocarbon linker elements of Formula 1 (See TABLE 1) that span more than one non-contiguous single α-helical turns of the composite peptide.

Not wishing to be bound to any specific peptide containing one or more intra-peptide hydrocarbon linker elements of the present embodiments, a generic peptide example containing one intra-peptide hydrocarbon linker element connecting two separate amino acids positioned 4 residues apart, or one α-helical turn (position i and position i+4), can have S-pentenylalanine (S5Ala) incorporated at each of the positions i and i+4 during solid-phase synthesis of the peptide before catalyzing ring-closing metathesis using Grubb's catalyst while the peptide is still resin-bound on the solid support. This will result in a peptide sequence containing the intra-peptide hydrocarbon linker element depicted below (SEQ ID NO: 23) positioned 4 residues apart:

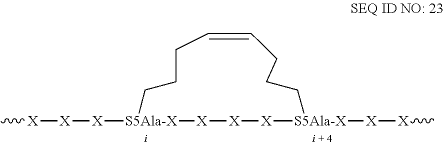

In some embodiments, one or more intra-peptide hydrocarbon linker elements are incorporated at amino acid positions that correlate with a double α-helical turn in a secondary structure of the composite peptide. In some embodiments, when the composite peptide comprises one or more non-contiguous double α-helical turns, the amino acid positions that correlate with a double α-helical turn of the composite peptide correspond to amino acid positions i and i+7 of the composite peptide, where i is the first amino acid position of the double α-helical turn and i+7 is the last amino acid position of the double α-helical turn, and wherein amino acid positions i and i+7 comprise alpha-alkenyl substituted amino acids, and where i and i+7 are positioned 7 residues apart (7 spaced).

Not wishing to be bound to any specific peptide containing one or more intra-peptide hydrocarbon linker elements of the present embodiments, a generic peptide example containing one intra-peptide hydrocarbon linker element connecting two separate amino acids positioned 7 residues apart, or two α-helical turns (position i and position i+7), can have R-octenylalanine (R8Ala) incorporated at position i and S-pentenylalanine (S5Ala) incorporated at position i+7 during solid-phase synthesis of the peptide before catalyzing ring-closing metathesis using Grubb's catalyst while the peptide is still resin-bound on the solid support. This will result in a peptide sequence containing the intra-peptide hydrocarbon linker elements depicted below (SEQ ID NO: 24) positioned 7 residues apart:

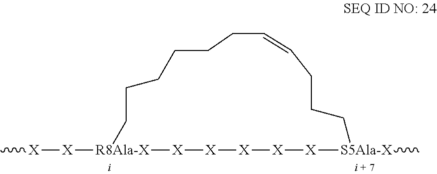

In some embodiments, one skilled in the art of solid-phase peptide synthesis can readily synthesize composite peptides comprising more than one intra-peptide hydrocarbon linker elements such that the composite peptide comprises more than one double α-helical turn. In some embodiments, the more than one double α-helical turns are non-contiguous, i.e., the more than one double α-helical turns do not share a substituted amino acid. For example, in some embodiments, the composite peptide can comprise one or more intra-peptide hydrocarbon linker elements of Formula 2 (See TABLE 1) that span more than one non-contiguous double α-helical turns of the composite peptide.

One skilled in the art of solid-phase peptide synthesis can readily synthesize peptides containing more than one intra-peptide hydrocarbon linker element of the present embodiments by incorporating α-alkenyl substituted amino acids at paired non-overlapping amino acid positions in the peptide, with each α-alkenyl substituted amino acid in the pair positioned a single α-helical turn apart (4 residues apart) or a double α-helical turn apart (7 residues apart) during solid-phase peptide synthesis before catalyzing ring-closing metathesis using Grubb's catalyst while the peptide is still resin-bound on the solid support. In some embodiments, single peptides can comprise more than one intra-peptide hydrocarbon linker element that span a single α-helical turn (4 residues apart), can contain hydrocarbon linker elements that span a double α-helical turn (7 residues apart), or can contain a combination of both a single α-helical turn (4 residues apart) and a double α-helical turn (7 residues apart) intra-peptide hydrocarbon linker elements.

Peptides containing one or more intra-peptide hydrocarbon linker elements of the present embodiments can be produced through solid-phase peptide synthesis utilizing commercially available Boc-or Fmoc-protected α-alkenyl substituted natural or non-natural amino acids in the (D) as well as (L), or the (R) as well as (S), stereochemical configuration. The Fmoc-protected α-alkenyl substituted amino acids and the resultant hydrocarbon linker element following ring-closing metathesis that may be used in the synthesis of the custom peptide derivatives of the present embodiments include, but are not limited to Table 1:

TABLE 1

| α-alkenyl Substituted Amino | α-alkenyl Substituted |
|---|---|
| Acid | Amino Acid |
| Peptide Position i | Peptide Position i + 4 |
| S-pentenylalanine (CAS: | S5Ala |
| 288617-73-2; S5Ala) | |

Hydrocarbon Linker Element Following Ring-Closing Metathesis

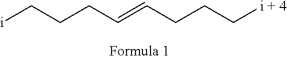

| Peptide Position i | Peptide Position i + 7 |
|---|---|
| R-octenylalanine (CAS: | S5Ala |
| 945212-26-0; R8Ala) | |

Hydrocarbon Linker Element Following Ring-Closing Metathesis

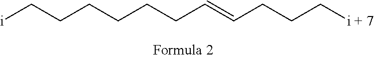

In some embodiments, an intra-peptide hydrocarbon linker can be further functionalized through one or more chemical reactions. In some embodiments, one or more carbon-carbon double bond(s) present in the intra-peptide hydrocarbon linker (e.g., Formula 1—Formula 2 in TABLE 1) can be utilized for organic chemical reactions to add one or more additional chemical functionalities. For example, alkene reactions may be utilized for custom peptide derivatives that contain one or more intra-peptide hydrocarbon linker elements of the present embodiments. Non-limiting examples of alkene reactions include hydroboration, oxymercuration, hydration, chlorination, bromination, addition of HF, HBr, HCl or HI, dihydroxylation, epoxidation, hydrogenation, and cyclopropanation. In some embodiments, one or more additional chemical functionalities of the intra-peptide hydrocarbon linker elements can be achieved subsequent to the alkene reaction. Non-limiting examples include covalent addition of one or more chemical group substituents, such as nucleophilic reactions with epoxide and hydroxyl groups, and the like. In some embodiments, alkene reactions may be utilized to attach biotin, radioisotopes, therapeutic agents (non-limiting examples include rapamycin, vinblastine, taxol, etc.), non-protein fluorescent chemical groups (non-limiting examples include FITC, hydrazide, rhodamine, maleimide, etc.), and protein fluorescent groups (non-limiting examples include GFP, YFP, mCherry, etc.) to one or more inter-and/or intra-peptide hydrocarbon linker elements of the present embodiments.

Non-limiting examples of composite peptides comprising one or more intra-peptide hydrocarbon linker elements are provided in TABLE 2. The examples in TABLE 2 are not limiting with respect to any specific α-alkenyl substituted amino acid useful for the synthesis of single α-helical turn (4 spaced) and/or double α-helical turn (7 spaced) intra-peptide hydrocarbon linker elements of the present embodiments and/or to any specific amino acid stereochemical configuration (e.g., (D) stereochemical configuration denoted with “d” in TABLE 2) in the custom peptide derivatives of the present embodiments.

TABLE 2

| | SEQ |
|---|---|
| | ID |
| | NO: |
| {S5Ala}-I-K-E-{S5Ala}-L-Q-R-F-I-H-I-V-Q-S-I- | 11 |
| I-N-T-S | |
| I-K-E-F-L-Q-R-{S5Ala}-I-H-I-{S5Ala}-Q-S-I-I- | 12 |
| N-T-S | |
| I-K-E-F-L-Q-R-{R8Ala}-I-H-I-V-Q-S-{S5Ala}-I- | 13 |
| N-T-S | |
| I-K-E-F-L-Q-R-F-I-H-I-{S5Ala}-Q-S-I-{S5A1a}- | 14 |
| N-T-S | |
| I-K-E-F-L-Q-R-F-I-H-I-{R8Ala}-Q-S-I-I-N-T- | 15 |
| {S5Ala} | |
| {S5Ala₁}-I-K-E-{S5Ala₁}-L-Q-R-{S5Ala₂}-I-H-I- | 16 |
| {S5Ala₂}-Q-S-I-I-N-T-S | |
| {S5Ala₁}-I-K-E-{S5Ala₁}-L-Q-R-{R8Ala₂}-I-H-I- | 17 |
| V-Q-S-{S5Ala₂}-I-N-T-S | |
| {S5Ala₁}-I-K-E-{S5Ala₁}-L-Q-R-F-I-H-I- | 18 |
| {S5Ala₂}-Q-S-I-{S5Ala₂}-N-T-S | |
| {S5Ala₁}-I-K-E-{S5Ala₁}-L-Q-R-F-I-H-I- | 19 |
| {R8Ala₂}-Q-S-I-I-N-T-{S5Ala₂} | |
| {S5Ala₁}-I-K-E-{S5Ala₁}-L-Q-R-{S5Ala₂}-I-H-I- | 20 |
| {S5Ala₂}-Q-S-I-I-{dN}-{dT}-{dS} | |
| {S5Ala₁}-I-K-E-{S5Ala₁}-L-Q-R-{R8Ala₂}-I-H-I- | 21 |
| V-Q-S-{S5Ala₂}-I-{dN}-{dT}-{dS} | |
| {S5Ala₁}-I-K-E-{S5Ala₁}-L-Q-R-F-I-H-I- | 22 |
| {S5Ala₂}-Q-S-I-{S5Ala₂}-{dN}-{dT}-{dS} | |

*Subscript denotes corresponding pairs of hydrocarbon-linked α-alkenyl substituted amino acids

In some embodiments, the therapeutic compound can be an antibody. The antibody can be developed to target a [symbol] c-cytokine, such as IL-2 or IL-15, or to a specific protein receptor whose activity and/or abundance is directly modulated by cytokine signaling, such as the transmembrane glycoprotein CD8 or proteins of the NKG2 C-type lectin receptor family, both of which are expressed on T-lymphocytes.

Some embodiments also relate to polynucleotides comprising nucleotide sequences encoding the peptides and antibodies of the present invention. “Nucleotide sequence,” “polynucleotide,” or “nucleic acid” can be used interchangeably, and are understood to mean either double-stranded DNA, a single-stranded DNA or products of transcription of the said DNAs (c.g., RNA molecules). Polynucleotides can be administered to cells or subjects and expressed by the cells or subjects, rather than administering the peptides themselves. Several embodiments also relate to genetic constructs comprising a polynucleotide sequence encoding the peptides of the present invention. Genetic constructs can also contain additional regulatory elements such as promoters and enhancers and, optionally, selectable markers.

### Methods of Treating C-Cytokine Mediated Diseases

Several embodiments relate to the use of therapeutic compounds, such as [symbol] c-antagonist peptides, cytokine targeted antibodies, and/or antibodies targeting a specific protein receptor whose activity and/or abundance is directly modulated by cytokine signaling in the treatment of [symbol] c-cytokine mediated diseases. Use of the therapeutic compounds according to the present embodiments allows for flexibility in the design and combination, which enables more comprehensive outcomes that would not be accomplished by conventional strategies employing small-molecule chemical inhibitors or anti-cytokine receptor antibodies.

Described herein is a novel method of modulating the action of [symbol] c-family cytokines. Such manipulations can yield effective methods of clinical interventions in treating autoimmune diseases such as alopecia, and alopecia associated disorders.

In some embodiments, compositions, methods, and kits for inhibiting, ameliorating, reducing a severity of, treating, delaying the onset of, or preventing at least one alopecia related disorder are described. In some embodiments, the therapeutic compounds described herein may be used for inhibiting, ameliorating, reducing a severity of, treating, delaying the onset of, or preventing one or more of alopecia arcata, alopecia totalis, alopecia subtotalis, alopecia universalis, alopecia diffusa, ophiasis-type alopecia areata, and other immune-mediated diseases associated with alopecia such as lichen planus, lichen sclerosus, lichen sclerosus et atrophicus, atopy, atopic dermatitis, psoriasis, psoriasis vugaris, psoriasis capitis, psoriasis guttate, psoriasis inversa, psoriatic arthritis, eczema, pemphigus, pemphigus vulgaris, pemphigus foliaceus, pemphigus vegetans, pemphigus erythematosus, mucous membrane pemphigoid, scarring mucous membrane pemphigoid, bullous pemphigoid, myasthenia gravis, thyroid disorders, Hashimoto's thyroiditis, hypothyroidism, endemic goiter, Addison's disease, morphea scleroderma, urticaria, prurigo, rosacea vitiligo, vitiligo, and graft-versus-host disease (GvHD).

Several embodiments relate to therapeutic compounds that would modulate the signaling of all or selective members of the [symbol] c-cytokines. In some embodiments, therapeutic compounds selectively modulate the signaling of individual [symbol] c-cytokine family members. In other embodiments, therapeutic compounds can comprehensively modulate the signaling of all [symbol] c-cytokine family members (Simul-Block). In some embodiments, therapeutic compounds can selectively modulate the signaling of subsets of the [symbol] c-cytokines. Not wishing to be bound by a particular theory, the therapeutic compounds can modulate the function of all or selective members of the [symbol] c-cytokines by diminishing the binding of [symbol] c-cytokines to the [symbol] c-subunit, for example, as a competitive inhibitor, or by modulating the activity and/or abundance of a specific protein receptor that is itself directly modulated by [symbol] c-cytokine signaling.

Several members of the [symbol] c-cytokine family have been implicated as being involved in alopecia disease progression. Alopecia is an immune-mediated disorder of the skin where there exists a T-cell hyperproliferative environment supporting T-cell targeting of hair follicle autoantigens ultimately resulting in hair loss. IL-2 and IL-15 expression is elevated in the lesional scalp biopsies of patients (Fuentes-Duculan et al. 2016 Exp Dermatol 4:282-6., Suarez-Farinas et al. 2015 J. Allergy Clin. Immunol. 136:1277-87., Waldmann 2013 J Investig Dermatol Symp Proc 16:S28-30.), and antibodies targeting the [symbol] c-cytokines IL-2 and IL-15 cach showed inhibitory activity in an alopecia mouse model, but none of the blocking antibodies alone could reverse the established disorder (Xing et al. 2014 Nat Med 9:1043-9.). IL-21 expression is elevated in the serum of alopecia patients versus healthy controls (Atwa et al. 2016 Int J Dermatol 55:666-72.), and genome-wide association studies have also positively correlated IL-2 and IL-21 with alopecia (Jagielska et al. 2012 J Invest Dermatol 132:2192-7, Petukhova et al. 2010 Nature 466:113-7.).

Vitiligo is an immune-mediated disorder of the skin associated with an influx of T-cells in the epidermis which results in melanocyte destruction and the appearance of white patches on the body surface. A recent study showed that blocking IL-15 signaling via antibody treatment was an effective therapeutic strategy in mice with established vitiligo (Richmond et al. 2018 Sci Transl Med 10:450). Interestingly, the antibody used in the study targeted CD122, the private cytokine-specific receptor subunit common to both IL-15 and IL-2. Indeed IL-2 expression has been shown to be elevated in the serum of localized vitiligo and generalized vitiligo patients and is positively correlated with disease severity (Sushama et al. 2018 J Cosmet Dermatol 00:1-5).

Pemphigoid and pemphigus are immune-mediated disorders of the skin characterized by the presence of large fluid-filled blisters on the body surface. In carly studies both pemphigoid and pemphigus blister fluid from human patients showed elevated IL-2 activity (Grando et al., 1989, Arch Dermatol. 125:925-30). Pemphigoid patients also displayed increased T-cell activation and elevated IL-2 levels (Schaller et al., 1990, Arch Dermatol. Res. 282:223-6). A separate study assessed the IL-15 level in both pemphigoid and pemphigus patients, and found that patients of either disease displayed increased IL-15 serum levels that were positively correlated with disease severity (D'Auria et al., 1999, Arch Dermatol. Res. 291:354-6).

Certain [symbol] c-cytokines have been shown to be positively correlated with psoriasis. Psoriasis is an immune-mediated disorder of the skin characterized by scaly red patches of extra skin cells that are often dry, itchy, and sometimes painful. The expression of IL-15 is elevated in skin lesions in psoriasis patients (Waldmann 2013 J Investig Dermatol Symp Proc 16:S28-30.). An IL-15 specific antibody, which potently interfered with the assembly of the IL-15 cytokine-receptor signaling complex, reduced the severity of the disease in a human psoriasis xenograft model (Villadsen et al., 2003, J. Clin. Invest. 112:1571-80). Another [symbol] c-cytokine, IL-21, has also been shown to be elevated in psoriatic patients and positively correlated with disease severity (Caruso et al. 2009 Cell Cycle 8: 3629-30., Botti et al. 2012 Curr Pharm Biotechnol 13: 1861-7., He et al. 2012, Br. J. Dermatol. 167:191-3). Blockade of the cytokine via anti-IL-21 antibody treatment resulted in a significant reduction in keratinocyte proliferation and inflammation in a human psoriasis xenograft mouse model (Caruso et al., 2009 Nat. Med. 15:1013-5).

Graft versus host disease (GvHD) can often result following hematopoietic cell transplantation in a patient as host cells are recognized as foreign entities by a donor's T-lymphocytes. GvHD manifests itself by host organ tissue damage as the donor-derived T-cells differentiate into CD4 and CD8 effector cells with the production of pro-inflammatory cytokines and direct CD8 T-cell cytotoxic effects. As it is well known that members of the [symbol] c-cytokine family are involved in the activation of CD4 and CD8 T-cells, the positive association of a number of [symbol] c-cytokines with GvHD pathogenesis has been reported. The prophylactic use of two IL-2 receptor antagonistic antibodies showed beneficial effects on GvHD in hematologic malignancy patients following donor-peripheral blood stem cell transplantation (Fang et al., 2012 Biol Blood Marrow Transplant. 18:754-62). Serum levels of IL-15 have also been shown to elevate sharply in GvHD patients within the first month of post-transplantation (Chik et al. 2003, J Pediatr Hematol Oncol. 25:960-4), and donor-derived IL-15 was shown to be critical for acute GvHD in a murine GvHD model (Blaser et al., 2005 Blood 105:894-901). Lastly, IL-21 expression was observed in skin and colon samples of GvHD patients, but not in GvHD-free control samples, and in GvHD murine models, serum IL-21 levels were elevated, and use of anti-human IL-21 antibodies reduced weight-loss and mortality associated with GvHD after administration (Hippen et al. 2012 Blood 119:619-28, Bucher et al. 2009 Blood 114:5375-84).

Several embodiments relate to the use of therapeutic antagonist peptides that selectively inhibit the activity of IL-15, either alone or in combination with the other [symbol] c-cytokine family members, as a therapeutic agent for alopecia and/or alopecia associated disorders. In some embodiments, custom derivative antagonist peptides that selectively inhibit IL-2, IL-15, IL-9, a combination of IL-2 and IL-15, a combination of IL-2 and IL-9, and/or a combination of IL-15 and IL-9 activities are used as a therapeutic agent for treating alopecia and/or alopecia associated diseases. In some embodiments, the effect of custom derivative antagonist peptides that selectively inhibit a combination of IL-2 and IL-15, a combination of IL-2 and IL-9, and/or a combination of IL-15 and IL-9 can be additive or synergistic. Several embodiments relate to the use of SEQ ID NO: 2 to treat alopecia and/or alopecia associated disorders. Several embodiments relate to the use of BNZ- [symbol] to treat alopecia and/or alopecia associated disorders. Several embodiments relate to the use of SEQ ID NO: 1 to treat alopecia and/or alopecia associated disorders.

Several embodiments relate to the use of therapeutic compounds, either alone or in combination, as a therapeutic agent for alopecia and/or alopecia associated disorders. In some embodiments, the therapeutic compound is SEQ ID NO: 2. In some embodiments, the therapeutic compound is BNZ- [symbol] . In some embodiments, the therapeutic compound is SEQ ID NO: 1. In some embodiments, the therapeutic compound is an anti-CD8 antibody. In some embodiments, the therapeutic compound is an anti-IL-2 antibody. In some embodiments, the therapeutic compound is an anti-IL-15 antibody. In some embodiments, the therapeutic compound is an anti-NKG2A antibody.

An additive effect is observed when the effect of a combination is equal to the sum of the effects of the individuals in the combination (e.g., the effect of a combination of two or more therapeutic compounds is equal to the sum of the effects of each therapeutic compound individually). A synergistic effect is observed when the effect of a combination is greater than the sum of the effects of the individuals in the combination (e.g., the effect of a combination of two or more therapeutic compounds is greater than the sum of the effects of each therapeutic compound individually). A synergistic effect is greater than an additive effect. Additive effect, synergistic effect, or both can occur in human patients, non-human patients, non-patient human volunteers, in vivo models, ex vivo models, in vitro models, etc.

In some embodiments, two or more therapeutic compounds disclosed herein can be used in combination. In some embodiments, two or more therapeutic compounds disclosed herein when used in combination yield an additive effect. In some embodiments, two or more therapeutic compounds disclosed herein when used in combination yield a synergistic effect. Synergistic effect can range from about >1 to about 100-fold. In some embodiments, the synergistic effect is about 2 to about 20-fold. In some embodiments, the synergistic effect is about 20 to about 100-fold. In some embodiments, the synergistic effect is from >1, 2, 3, 4, 5, 6, 7, 8, 9, 10, 20, 30, 40, 50, 60, 70, 80, 90, or 100-fold, or within a range defined by any two of the aforementioned values.

Another embodiment relates to the development of chemical compounds (non-peptide, non-protein) that have a spatial structure which resembles the 19-mer amino acid sequence I-K-E-F-L-Q-R-F-I-H-I-V-Q-S-I-I-N-T-S (SEQ ID NO: 1) and can fit into the pocket of the [symbol] c-subunit to structurally hinder the access of a [symbol] c-cytokine to the [symbol] c-subunit for binding. Some embodiments relate to the use of structurally similar chemical compounds as inhibitors of [symbol] c-cytokine activity. Such molecular mimicry strategy to further refine the development of synthetic compounds resembling in structure to existing biological peptide/proteins is described in Orzacz et al., 2009 Chem. Med. Chem. 4:146-160. Another embodiment relates to administration of chemical compounds (non-peptide, non-protein) that have a resembling 3D structure as the 19-mer amino acids sequence I-K-E-F-L-Q-R-F-I-H-I-V-Q-S-I-I-N-T-S (SEQ ID NO: 1) for inhibiting, ameliorating, reducing a severity of, treating, delaying the onset of, or preventing one or more alopecia and/or alopecia associated disorders.

Several embodiments relate to the administration of a peptide of amino acid sequence I-K-E-F-L-Q-R-F-I-H-I-V-Q-S-I-I-N-T-S (SEQ ID NO: 1) for inhibiting, ameliorating, reducing a severity of, treating, delaying the onset of, or preventing one or more alopecia and/or alopecia associated disorders. Another embodiment relates to the administration of derivative peptides of amino acid sequence I-K-E-F-L-Q-R-F-I-H-I-V-Q-S-I-I-N-T-S (SEQ ID NO: 1), wherein the amino acid sequence of the derivative peptide has similar physico-chemical properties as a peptide of the amino acid sequence I-K-E-F-L-Q-R-F-I-H-I-V-Q-S-I-I-N-T-S (SEQ ID NO: 1), but has distinct biological activity, for inhibiting, ameliorating, reducing a severity of, treating, delaying the onset of, or preventing one or more alopecia and/or alopecia associated disorders. Another embodiment relates to administration of a peptide of amino acid sequence I-K-E-F-L-Q-R-F-I-H-I-V-Q-S-I-I-N-T-S (SEQ ID NO: 1) conjugated to the N-and C-termini or to the side residues of existing biological proteins/peptides into patients for inhibiting, ameliorating, reducing a severity of, treating, delaying the onset of, or preventing one or more alopecia and/or alopecia associated disorders.

Several embodiments relate to the administration of a peptide of amino acid sequence I-K-E-F-L-Q-R-F-I-H-I-V-Q-S-I-I-N-T-S (SEQ ID NO: 1) for inhibiting, ameliorating, reducing a severity of, treating, delaying the onset of, or preventing one or more alopecia and/or alopecia associated disorders. Another embodiment relates to the administration of peptide derivatives of amino acid sequence I-K-E-F-L-Q-R-F-I-H-I-V-Q-S-I-I-N-T-S (SEQ ID NO: 1), wherein the amino acid sequence of the derivative peptide has similar physico-chemical properties as a peptide of the amino acid sequence I-K-E-F-L-Q-R-F-I-H-I-V-Q-S-I-I-N-T-S (SEQ ID NO: 1), but has distinct biological activity, for inhibiting, ameliorating, reducing a severity of, treating, delaying the onset of, or preventing one or more alopecia and/or alopecia associated disorders. Another embodiment relates to administration of a peptide of amino acid sequence I-K-E-F-L-Q-R-F-I-H-I-V-Q-S-I-I-N-T-S (SEQ ID NO: 1) conjugated to the N-and C-termini or to the side residues of existing biological proteins/peptides into patients for inhibiting, ameliorating, reducing a severity of, treating, delaying the onset of, or preventing one or more alopecia and/or alopecia associated disorders.

Several embodiments relate to administration of polyclonal and monoclonal antibodies raised against a peptide comprising of amino acid sequence I-K-E-F-L-Q-R-F-I-H-I-V-Q-S-I-I-N-T-S (SEQ ID NO: 1) into patients as an immunogen for inhibiting, ameliorating, reducing a severity of, treating, delaying the onset of, or preventing one or more alopecia and/or alopecia associated disorders. Another embodiment relates to administration of polyclonal and monoclonal antibodies that were raised against derivative peptides of amino acid sequence I-K-E-F-L-Q-R-F-I-H-I-V-Q-S-I-I-N-T-S (SEQ ID NO: 1), wherein the amino acid sequence of the derivative peptide has similar physico-chemical properties as a peptide of the amino acid sequence I-K-E-F-L-Q-R-F-I-H-I-V-Q-S-I-I-N-T-S (SEQ ID NO: 1), but has distinct biological activity, into patients as an immunogen for inhibiting, ameliorating, reducing a severity of, treating, delaying the onset of, or preventing one or more alopecia and/or alopecia associated disorders.

Several embodiments relate to administration of polyclonal and monoclonal antibodies raised against IL-2 into patients as an immunogen for inhibiting, ameliorating, reducing a severity of, treating, delaying the onset of, or preventing one or more alopecia and/or alopecia associated disorders. Another embodiment relates to administration of polyclonal and monoclonal antibodies raised against IL-15 into patients as an immunogen for inhibiting, ameliorating, reducing a severity of, treating, delaying the onset of, or preventing one or more alopecia and/or alopecia associated disorders. Another embodiment relates to administration of polyclonal and monoclonal antibodies raised against the transmembrane glycoprotein T-cell co-receptor CD8 into patients as an immunogen for inhibiting, ameliorating, reducing a severity of, treating, delaying the onset of, or preventing one or more alopecia and/or alopecia associated disorders. Another embodiment relates to administration of polyclonal and monoclonal antibodies raised against members of the C-type lectin receptor NKG2 family, for example NKG2D, NKG2A, into patients as an immunogen for inhibiting, ameliorating, reducing a severity of, treating, delaying the onset of, or preventing one or more alopecia and/or alopecia associated disorders.

### Administration of Therapeutic Compounds

The present embodiments also encompass the use of one or more therapeutic compounds selected from the group consisting of a [symbol] c-cytokine antagonist peptide, a [symbol] c-cytokine antagonist peptide derivative, anti-CD8 antibody, anti-IL-2 antibody, anti-IL-15 antibody, anti-NKG2A antibody, or a combination thereof [symbol] [symbol] for the manufacture of a medicament for inhibiting, ameliorating, reducing a severity of, treating, delaying the onset of, or preventing one or more alopecia and/or alopecia associated disorders. The present embodiments also encompass a pharmaceutical composition that includes one or more therapeutic compounds in combination with a pharmaceutically acceptable carrier. The pharmaceutical composition can include a pharmaceutically acceptable carrier and a non-toxic therapeutically effective amount of therapeutic compounds, or other compositions of the present embodiments.

The present embodiments provide methods of using pharmaceutical compositions comprising an effective amount of therapeutic compounds in a suitable diluent or carrier. A therapeutic compound of the present embodiments can be formulated according to known methods used to prepare pharmaceutically useful compositions. A therapeutic compound can be combined in admixture, either as the sole active material or with other known active materials, with pharmaceutically suitable diluents (c.g., phosphate, acetate, Tris-HCl), preservatives (e.g., thimerosal, benzyl alcohol, parabens), emulsifying compounds, solubilizers, adjuvants, and/or carriers such as bovine serum albumin.

In some embodiments, one or more compositions and kits comprising one or more of the therapeutic compounds disclosed herein are contemplated. In some embodiments, one or more compositions and kits are used for preventing and/or treating one or more diseases. In some embodiments, one or more compositions and kits are used for inhibiting, ameliorating, reducing a severity of, treating, delaying the onset of, or preventing one or more alopecia and/or an alopecia associated disorder.

In some embodiments, the one or more compositions and kits comprising one or more of the therapeutic compounds are administered to a subject in need thereof via any of the routes of administration provided herein. In some embodiments, the one or more compositions and kits comprises one or more of the therapeutic compounds at a therapeutically effective amount to modulate the signaling of one or more γc-cytokines selected from the group consisting of IL-2, IL-4, IL-7, IL-9, IL-15, and IL-21. In some embodiments, the one or more compositions and kits comprises one or more of the therapeutic compounds at a therapeutically effective amount to prevent and/or treat one or more diseases. In some embodiments, the one or more compositions and kits comprising one or more of the therapeutic compounds additionally comprise one or more pharmaceutically acceptable carriers, diluents, excipients or combinations thereof.

In some embodiments, one or more therapeutic compounds in the one or more compositions and kits are formulated as suitable for administration to a subject for preventing and/or treating one or more diseases. In some embodiments, one or more therapeutic compounds in the one or more compositions and kits are formulated as suitable for administration to a subject for preventing and/or treating alopecia and/or an alopecia associated disorder.

In some embodiments, one or more therapeutic compounds selected from the group consisting of SEQ ID NO: 1, SEQ ID NO: 2, an anti-CD8 antibody, an anti-IL-2 antibody, an anti-IL-15 antibody, and an anti-NKG2A antibody in the one or more compositions and kits are formulated as suitable for administration to a subject for preventing and/or treating one or more diseases. In some embodiments, one or more composite peptides selected from the group consisting of SEQ ID NO: 1, SEQ ID NO: 2, an anti-CD8 antibody, an anti-IL-2 antibody, an anti-IL-15 antibody, and an anti-NKG2A antibody in the one or more compositions and kits are formulated as suitable for administration to a subject for inhibiting, ameliorating, reducing a severity of, treating, delaying the onset of, or preventing one or more alopecia and/or an alopecia associated disorder.

In some embodiments, one or more derivatives of the one or more composite peptides selected from the group consisting of SEQ ID NO: 1, SEQ ID NO: 2; and an anti-CD8 antibody, an anti-IL-2 antibody, an anti-IL-15 antibody, and an anti-NKG2A antibody in the one or more compositions and kits are formulated as suitable for administration to a subject for preventing and/or treating one or more diseases. In some embodiments, one or more derivatives of the one or more composite peptides selected from the group consisting of SEQ ID NO: 1, SEQ ID NO: 2; and an anti-CD8 antibody, an anti-IL-2 antibody, an anti-IL-15 antibody, and an anti-NKG2A antibody in the one or more compositions and kits are formulated as suitable for administration to a subject for inhibiting, ameliorating, reducing a severity of, treating, delaying the onset of, or preventing one or more alopecia and/or an alopecia associated disorder.

The terms “disease,” “disorder,” and “biological condition” can be used interchangeably when referring to “inhibiting, ameliorating, reducing a severity of, treating, delaying the onset of, or preventing one or more diseases” provided in accordance with the present embodiments.

In some embodiments, the one or more derivatives of the one or more composite peptides comprise amino acid sequences that shares about 50% to about 99% identity with the one or more composite peptides. In some embodiments, the one or more derivatives of the one or more composite peptides comprise amino acid sequences that shares 50%, 50-60%, 60-70%, 70-80%, 80%, 90%, 95%, 97%, 98%, 99% or 99.8% identity with the one or more composite peptides, or within a range defined by any two of the aforementioned values.

In some embodiments, one or more alopecia associated disorder is selected from the group consisting of alopecia areata, alopecia totalis, alopecia subtotalis, alopecia universalis, alopecia diffusa, ophiasis-type alopecia areata, lichen planus, lichen sclerosus, lichen sclerosus et atrophicus, atopy, atopic dermatitis, psoriasis, psoriasis vugaris, psoriasis capitis, psoriasis guttate, psoriasis inversa, psoriatic arthritis, eczema, pemphigus, pemphigus vulgaris, pemphigus foliaceus, pemphigus vegetans, pemphigus erythematosus, mucous membrane pemphigoid, scarring mucous membrane pemphigoid, bullous pemphigoid, myasthenia gravis, thyroid disorders, Hashimoto's thyroiditis, hypothyroidism, endemic goiter, Addison's disease, morphea scleroderma, urticaria, prurigo, rosacea vitiligo, vitiligo, and graft-versus-host disease (GvHD).

Suitable carriers and their formulations are described in Remington's Pharmaceutical Sciences, 16^{th} ed. 1980 Mack Publishing CO, and Overview of Antibody Drug Delivery (Awwad et al. 2018 Pharmaceutics 10:83). Additionally, such compositions can contain a therapeutic compound complexed with polyethylene glycol (PEG), metal ions, or incorporated into polymeric compounds such as polyacetic acid, polyglycolic acid, hydrogels etc., or incorporated into liposomes, microemulsions, micelles, unilamellar or multilamellar vesicles, erythrocyte ghosts, or spheroblasts. Such compositions will influence the physical state, solubility, stability, rate of in vivo release, and rate of in vivo clearance of a therapeutic compound. A therapeutic compound can be conjugated to antibodies against cell-specific antigens, receptors, ligands, or coupled to ligands for tissue-specific receptors.

Methods of administrating therapeutic compounds of the present embodiments may be selected as appropriate, depending on factors, such as the type of diseases, the condition of subjects, and/or the site to be targeted. The therapeutic compounds can be administered topically, orally, parenterally, rectally, or by inhalation. Topical administration of therapeutic compounds can be achieved through formulation into lotions, liniments (balms), solutions, ointments, creams, pastes, gels, or other suitable topical delivery systems as appropriate (Gupta et al. 2016 Indo Amer J Pharm Res 6:6353-69.). Topical formulation components can include emollient and/or stiffening agents such as cetyl alcohol, cetyl ester wax, carnauba wax, lanolin, lanolin alcohols, paraffin, petrolatum, polyethylene glycol, stearic acid, stearyl alcohol, white or yellow wax; emulsifying and/or solubilizing agents such as polysorbate 20, polysorbate 80, polysorbate 60, poloxamer, sorbitan monostearate, sorbitan monooleate, sodium lauryl sulfate, propylene glycol monostearate; humectants such as glycerin, propylene glycol, polyethylene glycol; thickening/gelling agents such as carbomer, methyl cellulose, sodium carboxyl methyl cellulose, carrageenan, colloidal silicon dioxide, guar gum, hydroxypropyl cellulose, hydroxypropyl methyl cellulose, gelatin, polyethylene oxide, alginic acid, sodium alginate, fumed silica; preservative agents such as benzoic acid, propyl paraben, methyl paraben, imidurea, sorbic acid, potassium sorbate, benzalkonium chloride, phenyl mercuric acetate, chlorobutanol, phenoxyethanol; permeation enhancing agents such as propylene glycol, ethanol, isopropyl alcohol, oleic acid, polyethylene glycol; antioxidant agents such as butylated hydroxyanisole, butylated hydroxytoluene; buffering agents such as citric acid, phosphoric acid, sodium hydroxide, monobasic sodium phosphate; and vehicle agents such as purified water, propylene glycol, hexylene glycol, oleyl alcohol, propylene carbonate, and mineral oil (Chang et al. 2013 AAPS J 15:41-52.). Oral formulation components can include fatty acids and derivatives such as lauric acid, caprylic acid, oleic acid; bile salts such as sodium cholate, sodium deoxycholate, sodium taurodeoxycholate, sodium glycocholate; chelators such as citric acid, sodium salicylate; alkylglycoside containing polymers, cationic polymers, anionic polymers, and nanoparticles; and surfactants such as sodium dodecyl sulfate, sodium laurate dodecylmaltoside, polaxamer, sodium myristate, sodium laurylsulfate, quillayasaponin, and sucrose palmitate (Liu et al. 2018 Expert Opin Drug Del 15:223-33., Aguirre et al. 2016 Adv Drug Deliv Rev 106:223-41.). The term “parenteral” includes subcutaneous injections, intravenous, intramuscular, intraperitoneal, intracisternal injection, or infusion techniques. These compositions will typically include an effective amount of a therapeutic compound, alone or in combination with an effective amount of any other active material. Several non-limiting routes of administrations are possible including parenteral, subcutaneous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

The one or more therapeutic compounds disclosed herein can be administered at any dose, via any of the routes of administration, and at any frequency of administration as determined by one of ordinary skill in the art based on various parameters. Non-limiting examples of which include the condition being treated, the severity of the condition, patient compliance, efficacy of treatment, side effects, etc.

The amount of the therapeutic compound contained in pharmaceutical compositions of the present embodiments, dosage form of the pharmaceutical compositions, frequency of administration, and the like may be selected as appropriate, depending on factors, such as the type of diseases, the condition of subjects, and/or the site to be targeted. Such dosages and desired drug concentrations contained in the compositions may vary affected by many parameters, including the intended use, patient's body weight and age, and the route of administration. Pilot studies will first be conducted using animal studies and the scaling to human administration will be performed according to art-accepted practice.

In one embodiment, host cells that have been genetically modified with a polynucleotide encoding at least one therapeutic compound are administered to a subject for inhibiting, ameliorating, reducing a severity of, treating, delaying the onset of, or preventing one or more alopecia and/or an alopecia associated disorder. The polynucleotide is expressed by the host cells, thereby producing the therapeutic compound within the subject. Preferably, the host cells are allogeneic or autogeneic to the subject.

In a further aspect, the one or more therapeutic compounds selected from the group consisting of a [symbol] c-cytokine antagonist peptide, a [symbol] c-cytokine antagonist peptide derivative, anti-CD8 antibody, anti-IL-2 antibody, anti-IL-15 antibody, anti-NKG2A antibody, or a combination thereof can be used in combination with other therapies, for example, therapies inhibiting cancer cell proliferation and growth. The phrase “combination therapy” embraces the administration of the one or more therapeutic compounds selected from the group consisting of a [symbol] c-cytokine antagonist peptide, a [symbol] c-cytokine antagonist peptide derivative, anti-CD8 antibody, anti-IL-2 antibody, anti-IL-15 antibody, anti-NKG2A antibody, or a combination thereof and one or more additional therapeutic agent as part of a specific treatment regimen intended to provide a beneficial effect from the co-action of these therapeutic agents. Administration of these therapeutic agents in combination typically is carried out over a defined time period (usually minutes, hours, days or weeks depending upon the combination selected).

A combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by an appropriate route including, but not limited to, oral routes, intravenous routes, intramuscular routes, and direct absorption through mucous membrane tissues. There therapeutic agents can be administered by the same route or by different routes. The sequence in which the therapeutic agents are administered is not narrowly critical.

Combination therapy also can embrace the administration of the therapeutic agents as described above in further combination with other biologically active ingredients (such as, but not limited to, a second and different therapeutic agent) and non-drug therapies (such as, but not limited to, surgery or radiation treatment). Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporarily removed from the administration of the therapeutic agents, perhaps by days or even weeks.

In certain embodiments, the one or more therapeutic compounds selected from the group consisting of a [symbol] c-cytokine antagonist peptide, a [symbol] c-cytokine antagonist peptide derivative, anti-CD8 antibody, anti-IL-2 antibody, anti-IL-15 antibody, anti-NKG2A antibody, or a combination thereof can be administered in combination with at least one anti-proliferative agent selected from the group consisting of chemotherapeutic agent, an antimetabolite, and antitumorgenic agent, and antimitotic agent, and antiviral agent, and antineoplastic agent, an immunotherapeutic agent, and a radiotherapeutic agent.

In certain embodiments, the one or more therapeutic compounds selected from the group consisting of a [symbol] c-cytokine antagonist peptide, a [symbol] c-cytokine antagonist peptide derivative, anti-CD8 antibody, anti-IL-2 antibody, anti-IL-15 antibody, anti-NKG2A antibody, or a combination thereof can be administered in combination with at least one anti-inflammatory agent selected from the group consisting of steroids, corticosteroids, and nonsteroidal anti-inflammatory drugs.

Also provided are kits for performing any of the above methods. Kits may include the one or more therapeutic compounds selected from the group consisting of a [symbol] c-cytokine antagonist peptide, a [symbol] c-cytokine antagonist peptide derivative, anti-CD8 antibody, anti-IL-2 antibody, anti-IL-15 antibody, anti-NKG2A antibody, or a combination thereof according to the present embodiments. In some embodiments, the kit may include instructions. Instructions may be in written or pictograph form, or may be on recorded media including audio tape, audio CD, video tape, DVD, CD-ROM, or the like. The kits may comprise packaging.

### Additional Embodiments

In some embodiments of the method, the composite peptide comprises the amino acid sequence D/E-F-L-E/Q/N-S/R-X-I/K-X-L/I-X-Q (SEQ ID NO: 2), wherein X denotes any amino acid. In some embodiments of the method, the composite peptide derivative shares at least about 50% identity with a peptide of SEQ ID NO: 2. In some embodiments of the method, the composite peptide derivative shares at least about 90% identity with a peptide of SEQ ID NO: 2. In some embodiments of the method, the composite peptide derivative shares at least about 95% identity with a peptide of SEQ ID NO: 2. In some embodiments of the method, the composite peptide and the composite peptide derivative have similar physico-chemical properties but distinct biological activities.

In some embodiments of the method, the composite peptide comprises the amino acid sequence I-K-E-F-L-Q-R-F-I-H-I-V-Q-S-I-I-N-T-S (SEQ ID NO: 1) (BNZ- [symbol] ). In some embodiments of the method, the composite peptide derivative shares at least about 50% identity with a peptide of SEQ ID NO: 1. In some embodiments of the method, the composite peptide derivative shares at least about 90% identity with a peptide of SEQ ID NO: 1. In some embodiments of the method, the composite peptide derivative shares at least about 95% identity with a peptide of SEQ ID NO: 1. In some embodiments of the method, the composite peptide and the composite peptide derivative have similar physico-chemical properties but distinct biological activities.

In some embodiments of the method, the composite peptide comprises the amino acid sequence I-K-E-F-L-Q-R-F-I-H-I-V-Q-S-I-I-N-T-S (SEQ ID NO: 1). In some embodiments of the method, the composite peptide derivative shares at least about 50% identity with a peptide of SEQ ID NO: 1. In some embodiments of the method, the composite peptide derivative shares at least about 90% identity with a peptide of SEQ ID NO: 1. In some embodiments of the method, the composite peptide derivative shares at least about 95% identity with a peptide of SEQ ID NO: 1. In some embodiments of the method, the composite peptide and the composite peptide derivative have similar physico-chemical properties but distinct biological activities.

In some embodiments of the method, the composite peptide or composite peptide derivative inhibits the activity of one or more [symbol] c-cytokines. In some embodiments of the method, the one or more γc-cytokines are selected from the group consisting of IL-2, IL-4, IL-7, IL-9, IL-15 and IL-21. In some embodiments of the method, the composite peptide or composite peptide derivative inhibits the activity of IL-2, IL-15 and IL-9. In some embodiments of the method, the composite peptide or composite peptide derivative inhibits the activity of IL-2 and IL-15. In some embodiments of the method, the composite peptide or composite peptide derivative inhibits the activity of IL-15 and IL-9. In some embodiments of the method, the composite peptide or composite peptide derivative inhibits the activity of IL-15 and IL-21.

In some embodiments, the composite peptide or composite peptide derivative comprises a signal peptide. In some embodiments, the composite peptide or composite peptide derivative is further conjugated to one or more additional moieties at the N terminus, C terminus or a side residue of the composite peptide or composite peptide derivative. In some embodiments of the composite peptide or composite peptide derivative, the one or more additional moieties are selected from the group consisting of bovine serum albumin (BSA), albumin, Keyhole Limpet Hemocyanin (KLH), Fc region of IgG, a biological protein that functions as scaffold, an antibody against a cell-specific antigen, a receptor, a ligand, a metal ion, and Poly Ethylene Glycol (PEG).

In some embodiments, the composite peptide or composite peptide derivative comprises at least two alpha-alkenyl substituted amino acids, and wherein the at least two alpha-alkenyl substituted amino acids are linked via at least one intra-peptide hydrocarbon linker element is provided. In some embodiments of the composite peptide, the at least two alpha-alkenyl substituted amino acids are linked to form the at least one intra-peptide hydrocarbon linker element by ring closing metathesis, wherein the ring closing metathesis is catalyzed by Grubb's catalyst.

In some embodiments, an amino acid in the composite peptide is selected from the group consisting of natural amino acids, non-natural amino acids, (D) stereochemical configuration amino acids, (L) stereochemical configuration amino acids, (R) stereochemical configuration amino acids and (S) stereochemical configuration amino acids, and wherein the at least two alpha-alkenyl substituted amino acids are selected from S-pentenylalanine (CAS: 288617-73-2; S5Ala) and R-octenylalanine (CAS: 945212-26-0; R8Ala).

In some embodiments of the composite peptide, the at least two alpha-alkenyl substituted amino acids linked by the at least one intra-peptide hydrocarbon are separated by n-2 amino acids, wherein n represents the number of amino acids encompassed by the intra-peptide linkage.

In some embodiments of the composite peptide, when the at least two alpha-alkenyl substituted amino acids linked by the at least one intra-peptide hydrocarbon are separated by three amino acids, the at least one intra-peptide hydrocarbon linker element spans a single α-helical turn of the composite peptide.

In some embodiments of the composite peptide, when the composite peptide comprises one or more non-contiguous single α-helical turns, the amino acid positions that correlate with a single α-helical turn of the composite peptide correspond to amino acid positions i and i+4 of the composite peptide, where i is the first amino acid position of the single α-helical turn and i+4 is the last amino acid position of the single α-helical turn, and wherein amino acid positions i and i+4 comprise alpha-alkenyl substituted amino acids. In some embodiments of the composite peptide, when the alpha-alkenyl substituted amino acid at position i is S5Ala, the alpha-alkenyl substituted amino acid at position i+4 is also S5Ala, the hydrocarbon linker element formed by the ring-closing metathesis is represented by Formula 1.

In some embodiments of the composite peptide, when the at least two alpha-alkenyl substituted amino acids linked by the at least one intra-peptide hydrocarbon are separated by six residues, the at least one intra-peptide hydrocarbon linker element spans a double α-helical turn of the composite peptide.

In some embodiments of the composite peptide, when the composite peptide comprises one or more non-contiguous double α-helical turns, the amino acid positions that correlate with a double α-helical turn of the composite peptide correspond to amino acid positions i and i+7 of the composite peptide, where i is the first amino acid position of the double α-helical turn and i+7 is the last amino acid position of the double α-helical turn, and wherein amino acid positions i and i+7 comprise alpha-alkenyl substituted amino acids. In some embodiments of the composite peptide, when the alpha-alkenyl substituted amino acid at position i is R8Ala, the alpha-alkenyl substituted amino acid at position i+7 is S5Ala, the hydrocarbon linker element formed by the ring-closing metathesis is represented by Formula 2.

In some embodiments, the composite peptide comprises amino acid sequences of at least two interleukin (IL) protein gamma-c-box D-helix regions, wherein the composite peptide comprises the amino acid sequence I-K-E-F-L-Q-R-F-I-H-I-V-Q-S-I-I-N-T-S (SEQ ID NO: 1), and wherein the composite peptide comprises at least two alpha-alkenyl substituted amino acids, and wherein the at least two alpha-alkenyl substituted amino acids are linked via at least one intra-peptide hydrocarbon linker element.

In some embodiments, the composite peptide comprises amino acid sequences of at least two interleukin (IL) protein gamma-c-box D-helix regions, wherein the composite peptide comprises the amino acid sequence I-K-E-F-L-Q-R-F-I-H-I-V-Q-S-I-I-N-T-S (SEQ ID NO: 1), and wherein the composite peptide comprises at least two alpha-alkenyl substituted amino acids, and wherein the at least two alpha-alkenyl substituted amino acids are linked via at least one intra-peptide hydrocarbon linker element.

In some embodiments of the composite peptide, the one or more carbon-carbon double bonds present in the intra-peptide hydrocarbon linker are utilized for one or more organic chemical reactions to add one or more additional chemical functionalities. In some embodiments of the composite peptide, the one or more organic chemical reactions comprises an alkene reaction. In some embodiments of the composite peptide, the alkene reaction is selected from the group consisting of hydroboration, oxymercuration, hydration, chlorination, bromination, addition of HF, HBr, HCl or HI, dihydroxylation, cpoxidation, hydrogenation, and cyclopropanation. In some embodiments of the composite peptide, one or more additional chemical functionalities can be added subsequent to the alkene reaction wherein the one or more additional chemical functionalities comprise a covalent addition of one or more chemical group substituents, wherein the covalent addition of one or more chemical group substituents comprises nucleophilic reactions with epoxide and hydroxyl groups. In some embodiments of the composite peptide, the one or more additional chemical functionalities are selected from the group consisting of biotin, radioisotopes, therapeutic agents, rapamycin, vinblastine, taxol, non-protein fluorescent chemical groups, FITC, hydrazide, rhodamine, maleimide, protein fluorescent groups, GFP, YFP, and mCherry.

In some embodiments, a pharmaceutical composition is provided. In some embodiments, the pharmaceutical composition comprises a therapeutically effective amount of a peptide conjugate or a derivative thereof, and a pharmaceutically acceptable carrier, diluent, excipient or combination thereof, wherein the peptide conjugate or the derivative thereof modulates the activity of two or more γc-cytokines selected from the group consisting of IL-2, IL-4, IL-7, IL-9, IL-15, and IL-21, wherein the peptide conjugate comprises the amino acid sequence D/E-F-L-E/Q/N-S/R-X-I/K-X-L/I-X-Q (SEQ ID NO: 2), wherein X denotes any amino acid, and wherein the derivative thereof comprises a peptide sequence sharing at least 90% identity with the amino acid sequence of SEQ ID NO: 2.

In some embodiments, a pharmaceutical composition is provided. In some embodiments, the pharmaceutical composition comprises a therapeutically effective amount of a peptide conjugate or a derivative thereof, and a pharmaceutically acceptable carrier, diluent, excipient or combination thereof, wherein the peptide conjugate or the derivative thereof modulates the activity of two or more γc-cytokines selected from the group consisting of IL-2, IL-4, IL-7, IL-9, IL-15, and IL-21, wherein the peptide conjugate comprises the amino acid sequence I-K-E-F-L-Q-R-F-I-H-I-V-Q-S-I-I-N-T-S (SEQ ID NO: 1), and wherein the derivative thereof comprises a peptide sequence sharing at least 90% identity with the amino acid sequence of SEQ ID NO: 1.

In some embodiments, a pharmaceutical composition is provided. In some embodiments, the pharmaceutical composition comprises a therapeutically effective amount of a peptide conjugate or a derivative thereof, and a pharmaceutically acceptable carrier, diluent, excipient or combination thereof, wherein the peptide conjugate or the derivative thereof modulates the activity of two or more γc-cytokines selected from the group consisting of IL-2, IL-4, IL-7, IL-9, IL-15, and IL-21, wherein the peptide conjugate comprises the amino acid sequence I-K-E-F-L-Q-R-F-I-H-I-V-Q-S-I-I-N-T-S (SEQ ID NO: 1), and wherein the derivative thereof comprises a peptide sequence sharing at least 90% identity with the amino acid sequence of SEQ ID NO: 1.

In some embodiments of the pharmaceutical composition, the peptide conjugate or the derivative thereof inhibits the activity of two or more [symbol] c-cytokines selected from the group consisting of IL-2, IL-4, IL-7, IL-9, IL-15, and IL-21. In some embodiments of the pharmaceutical composition, the peptide conjugate or the derivative thereof further comprises an additional conjugate at the N termini, C termini or a side residues thereof.

In some embodiments of the pharmaceutical composition, the peptide conjugate or the derivative thereof further comprises a signal peptide. In some embodiments, the pharmaceutical composition further comprises a protein that stabilizes the structure of the peptide conjugate or the derivative thereof and improves its biological activity, wherein the protein is selected from the group consisting of bovine serum albumin (BSA), albumin, Fc region of immunoglobulin G (IgG), biological proteins that function as scaffold, Poly Ethylene Glycol (PEG), and derivatives thereof. In some embodiments of the pharmaceutical composition, the derivative thereof comprises a peptide sequence sharing at least 95% identity with the amino acid sequence of SEQ ID NO: 2. In some embodiments of the pharmaceutical composition, the derivative thereof comprises a peptide sequence sharing at least 95% identity with the amino acid sequence of SEQ ID NO: 1.

In some embodiments, a method of treating an alopecia associated disease is provided. In some embodiments, the method comprises administering a pharmaceutical composition provided herein to a subject in need thereof, wherein the alopecia associated disease is selected from the group consisting of alopecia areata, alopecia totalis, alopecia subtotalis, alopecia universalis, alopecia diffusa, ophiasis-type alopecia areata, lichen planus, lichen sclerosus, lichen sclerosus et atrophicus, atopy, atopic dermatitis, psoriasis, psoriasis vugaris, psoriasis capitis, psoriasis guttate, psoriasis inversa, psoriatic arthritis, eczema, pemphigus, pemphigus vulgaris, pemphigus foliaceus, pemphigus vegetans, pemphigus erythematosus, mucous membrane pemphigoid, scarring mucous membrane pemphigoid, bullous pemphigoid, myasthenia gravis, thyroid disorders, Hashimoto's thyroiditis, hypothyroidism, endemic goiter, Addison's disease, morphea scleroderma, urticaria, prurigo, rosacea vitiligo, vitiligo, and graft-versus-host disease (GvHD).

In some embodiments, a kit for treating an alopecia associated disease in a patient is provided.

In some embodiments, the kit comprises a pharmaceutical composition, wherein the pharmaceutical composition comprises a therapeutically effective amount of a peptide conjugate, or a derivative thereof, and a pharmaceutically acceptable carrier, diluent, excipient or combination thereof, wherein the peptide conjugate or the derivative thereof modulates the activity of two or more γc-cytokines selected from the group consisting of IL-2, IL-4, IL-7, IL-9, IL-15, and IL-21, wherein the peptide conjugate comprises the amino acid sequence D/E-F-L-E/Q/N-S/R-X-I/K-X-L/I-X-Q (SEQ ID NO: 2), wherein X denotes any amino acid, and wherein the derivative thereof comprises a peptide sequence sharing at least 90% identity with the amino acid sequence of SEQ ID NO: 2.

In some embodiments, the kit comprises a pharmaceutical composition, wherein the pharmaceutical composition comprises a therapeutically effective amount of a peptide conjugate, or a derivative thereof, and a pharmaceutically acceptable carrier, diluent, excipient or combination thereof, wherein the peptide conjugate or the derivative thereof modulates the activity of two or more γc-cytokines selected from the group consisting of IL-2, IL-4, IL-7, IL-9, IL-15, and IL-21, wherein the peptide conjugate comprises the amino acid sequence I-K-E-F-L-Q-R-F-I-H-I-V-Q-S-I-I-N-T-S (SEQ ID NO: 1), and wherein the derivative thereof comprises a peptide sequence sharing at least 90% identity with the amino acid sequence of SEQ ID NO: 1.

In some embodiments, the kit comprises a pharmaceutical composition, wherein the pharmaceutical composition comprises a therapeutically effective amount of a peptide conjugate, or a derivative thereof, and a pharmaceutically acceptable carrier, diluent, excipient or combination thereof, wherein the peptide conjugate or the derivative thereof modulates the activity of two or more γc-cytokines selected from the group consisting of IL-2, IL-4, IL-7, IL-9, IL-15, and IL-21, wherein the peptide conjugate comprises the amino acid sequence I-K-E-F-L-Q-R-F-I-H-I-V-Q-S-I-I-N-T-S (SEQ ID NO: 1), and wherein the derivative thereof comprises a peptide sequence sharing at least 90% identity with the amino acid sequence of SEQ ID NO: 1.

In some embodiments of the kit, the condition is one or more of alopecia areata, alopecia totalis, alopecia subtotalis, alopecia universalis, alopecia diffusa, ophiasis-type alopecia arcata, lichen planus, lichen sclerosus, lichen sclerosus et atrophicus, atopy, atopic dermatitis, psoriasis, psoriasis vugaris, psoriasis capitis, psoriasis guttate, psoriasis inversa, psoriatic arthritis, eczema, pemphigus, pemphigus vulgaris, pemphigus foliaceus, pemphigus vegetans, pemphigus erythematosus, mucous membrane pemphigoid, scarring mucous membrane pemphigoid, bullous pemphigoid, myasthenia gravis, thyroid disorders, Hashimoto's thyroiditis, hypothyroidism, endemic goiter, Addison's disease, morphea scleroderma, urticaria, prurigo, rosacea vitiligo, vitiligo, or graft-versus-host disease (GvHD).

### Definitions

As used herein, the term “patient” or “subject” refers to the recipient of any of the embodiments of the composite peptides disclosed herein and includes all organisms within the kingdom animalia. In some embodiments, any vertebrate including, without limitation, humans and other primates (e.g., chimpanzees and other apes and monkey species), farm animals (c.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, etc.) are included. In preferred embodiments, the animal is within the family of mammals, such as humans, bovine, ovine, porcine, feline, buffalo, canine, goat, equine, donkey, deer, and primates. The most preferred animal is human. In some embodiments, the patient is a male or a female.

As used herein, the term “treat” or any variation thereof (e.g., , treatment, treating, etc.), refers to any treatment of a patient diagnosed with a biological condition, such as alopecia areata, alopecia totalis, alopecia subtotalis, alopecia universalis, alopecia diffusa, ophiasis-type alopecia areata, lichen planus, lichen sclerosus, lichen sclerosus et atrophicus, atopy, atopic dermatitis, psoriasis, psoriasis vugaris, psoriasis capitis, psoriasis guttate, psoriasis inversa, psoriatic arthritis, eczema, pemphigus, pemphigus vulgaris, pemphigus foliaceus, pemphigus vegetans, pemphigus erythematosus, mucous membrane pemphigoid, scarring mucous membrane pemphigoid, bullous pemphigoid, myasthenia gravis, thyroid disorders, Hashimoto's thyroiditis, hypothyroidism, endemic goiter, Addison's disease, morphea scleroderma, urticaria, prurigo, rosacea vitiligo, vitiligo, and graft-versus-host disease (GvHD).

The term treat, as used herein, includes: (i) preventing or delaying the presentation of symptoms associated with the biological condition of interest in an at-risk patient who has yet to display symptoms associated with the biological condition; (ii) ameliorating the symptoms associated with the biological condition of interest in a patient diagnosed with the biological condition; (iii) preventing, delaying, or ameliorating the presentation of symptoms associated with complications, conditions, or diseases associated with the biological condition of interest in either an at-risk patient or a patient diagnosed with the biological condition; (iv) slowing, delaying or halting the progression of the biological condition; and/or (v) preventing, delaying, slowing, halting or ameliorating the cellular events of inflammation; and/or (vi) preventing, delaying, slowing, halting or ameliorating the histological abnormalities and/or other clinical measurements of the biological condition.

The term “symptom(s)” as used herein, refers to common signs or indications that a patient is suffering from a specific condition or disease.

The term “effective amount,” as used herein, refers to the amount necessary to elicit the desired biological response. In accordance with the present embodiments, an effective amount of a [symbol] c-antagonist is the amount necessary to provide an observable effect in at least one biological factor for use in treating a biological condition.

“Recombinant DNA technology” or “recombinant” refers to the use of techniques and processes for producing specific polypeptides from microbial (e.g., bacterial, yeast), invertebrate (insect), mammalian cells or organisms (c.g., transgenic animals or plants) that have been transformed or transfected with cloned or synthetic DNA sequences to enable biosynthesis of heterologous peptides. Native glycosylation pattern will only be achieved with mammalian cell expression system. Prokaryotic expression systems lack the ability to add glycosylation to the synthesized proteins. Yeast and insect cells provide a unique glycosylation pattern that may be different from the native pattern.

A “nucleotide sequence” refers to a polynucleotide in the form of a separate fragment or as a component of a larger DNA construct that has been derived from DNA or RNA isolated at least once in substantially pure form, free of contaminating endogenous materials and in a quantity or concentration enabling identification, manipulation, and recovery of its component nucleotide sequences by standard molecular biology methods (as outlined in Current Protocols in Molecular Biology).

“Recombinant expression vector” refers to a plasmid comprising a transcriptional unit containing an assembly of (1) a genetic clement or elements that have a regulatory role in gene expression including promoters and enhances, (2) a structure or coding sequence that encodes the polypeptide according to the present embodiments, and (3) appropriate transcription and translation initiation sequence and, if desired, termination sequences. Structural elements intended for use in yeast and mammalian system preferably include a signal sequence enabling extracellular secretion of translated polypeptides by yeast or mammalian host cells.

“Recombinant microbial expression system” refers to a substantially homogenous monoculture of suitable hot microorganisms, for example, bacteria such as E. coli, or yeast such as S. cerevisiae, that have stably integrated a recombinant transcriptional unit into chromosomal DNA or carry the recombinant transcriptional unit as a component of a residual plasmid. Generally, host cells constituting a recombinant microbial expression system are the progeny of a single ancestral transformed cell. Recombinant microbial expression systems will express heterologous polypeptides upon induction of the regulatory elements linked to a structural nucleotide sequence to be expressed.

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although this invention has been disclosed in the context of certain embodiments and examples, those skilled in the art will understand that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure.

It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes or embodiments of the disclosed invention. Thus, it is intended that the scope of the present invention herein disclosed should not be limited by the particular disclosed embodiments described above.

It should be understood, however, that this detailed description, while indicating preferred embodiments of the invention, is given by way of illustration only, since various changes and modifications within the spirit and scope of the invention will become apparent to those skilled in the art.

### EXAMPLES

The following Examples are presented for the purposes of illustration and should not be construed as limitations.

### Example 1—Method for Assessing the Inhibitory Activity of C-Antagonist Peptide

The capacity of any custom derivative peptide prepared according to the present embodiments for inhibiting the action of one [symbol] c-cytokine family member is determined using mammalian cellular assays to measure their proliferative response to the [symbol] c-cytokine family member.

For each of the six [symbol] c-cytokines, indicator cell lines: NK92, a human NK cell line NK92 available by American Type Culture Collection (ATCC) (catalog #CRL-2407), CTLL-2, a murine CD8 T cells line available from ATCC, and PT-18, a murine mast cell line and its subclone PT-1800 [symbol] [symbol] [symbol] is transfected with human IL- [symbol] R [symbol] gene to make the cells responsive to IL-2 and IL-15 (Tagaya et al., 1996, EMBO J. 15:4928-39), and is used to quantitatively determine the [symbol] c-cytokine's growth-promoting activity (See Current protocols in Immunology from Wiley and Sons for a methodological reference). The indicator cells demonstrate semi-linear dose-dependent response when measured by a colorimetric WST-1 assay over a range of concentrations (See Clontech PT3946-1 and associated user manual, incorporated herein by reference, for a detailed description of the reagents and methods).

Once the appropriate doses of the cytokine that yield the 50% and 95% maximum response from the indicator cell line is determined, various concentrations (ranging from 1 pM to 10 [symbol] OM) of the purified or synthesized custom derivative peptide is added to each well containing the cytokine and indicator cells. The reduction in light absorbance at 450 nm is used as an indicator of inhibition of cytokine-stimulated cellular proliferation. Typically, the cells are stimulated by the cytokines such that the absorbance of the well containing indicator cell line and the cytokine is between 2.0 and 3.0, which is reduced to a range of 0.1 to 0.5 by the addition of inhibitory peptides.

### Example 2—The Selective Inhibition of the Growth-Promoting Activities of Certain C-Cytokines by BNZ-

Using PT-18 [symbol] [symbol] cells as described above, the ability of the BNZ- [symbol] peptide to specifically inhibit the growth-promoting activity of select [symbol] c-cytokines was determined (FIG. 3A). IL-3, a non- [symbol] c-cytokine that supports the growth of PT-18 [symbol] cells, was used as a negative control. Briefly, PT-18 [symbol] [symbol] cells were incubated either with two different dilutions of BNZ-peptide produced by HEK293T cells (1:20 or 1:60 dilution of the original supernatant of HEK293T cells transfected with a BNZ-πexpression construct) or without BNZ- [symbol] peptide in the presence of IL-3, IL-9, IL-15, or IL-4 (1 nM of each cytokine in the culture).

The growth-responses of the cells were determined 2 days after the introduction of BNZ- [symbol] peptide and the cytokine using the WST-1 assay. The growth-promoting activity of IL-3 (a non [symbol] c-cytokine) was not inhibited by BNZ- [symbol] . In contrast, the activity of IL-15 and IL-9 were significantly (p<0.01 Student's T test) reduced by the BNZ- [symbol] [symbol] peptide. Cellular proliferation stimulated by IL-4, another [symbol] c-cytokine, was not affected by the by the addition of BNZ-π peptide. Results for IL-3, IL-9, IL-15, and IL-4 are shown at FIG. 3A.

In a similar assay, the murine cell line CTTL2 was used. In this assay the cells were cultured with 0.5 nM of recombinant IL-2 in RPMI 10% fetal Calf Serum. To set up the proliferation assay, cells were washed from the cytokines 3 times. Cells were seeded at 1×10(5) cells per well of a 96-well plate with final concentration of 50 PM of IL-2 or IL-15. Various concentration of BNZ- [symbol] peptide (0.1, 1, and 10 μM) was added to each well. Cells were cultured for 20 hours and in the last 4 hours, 3H-thymidine was added to the plates. Cells were harvested and radioactivity measured to determine cell proliferation levels. The data are shown in FIG. 3B.

### Example 3—Method for Measuring Inhibition C-Cytokine Activity by Assaying 3H-Thymidine Incorporation of as a Marker of Cellular Proliferation

Inhibition of [symbol] c-cytokine-induced proliferation of an indicator cell population by antagonist custom derivative peptides is measured by the 3H-thymidine incorporation assay. Briefly, radiolabeled thymidine (1 microCi) is given to 20-50,000 cells undergoing proliferation in the presence of cytokines. The cell-incorporated radioactivity is measured by trapping cell-bound radioactivity to a glass-fiber filter using a conventional harvester machines (Example, Filtermate Universal Harvester from Perkin-Elmer), after which the radioactivity is measured using a b-counter (Example 1450, Trilux microplate scintillation counter).

### Example 4—Method for Measuring Inhibition C-Cytokine Activity by Assaying Incorporation of a Cell-Tracker Dye as a Marker of Cellular Proliferation

Indicator cells are incubated in the presence of a selected [symbol] c-cytokine or in the presence of a selected [symbol] c-cytokine and a selected custom derivative peptide. The cell population is then labeled in vitro using a cell-tracker dye, for example, CMFDA, C2925 from Invitrogen, and the decay of cellular green fluorescence at cach cellular division is monitored using a flow-cytometer (for example, Beckton-Dickinson FACScalibur). Typically, in response to [symbol] c-cytokine stimulation 7˜10 different peaks corresponding to the number of divisions that the cells have undergone will appear on the green fluorescence channel. Incubation of the cells with the selected [symbol] c-cytokine and antagonist custom derivative peptide reduces the number of peaks to only 1 to 3, depending on the degree of the inhibition.

### Example 5—Inhibition of Intracellular Signaling by Custom Peptide Derivative Antagonists

In addition to stimulating cellular proliferation, binding of the [symbol] c-cytokines to their receptors causes a diverse array of intracellular events. (Rochman et al. 2009 Nat. Rev. Immunol. 9:480-90, Pesu et al. 2005 Immunol. Rev. 203:127-142.) Immediately after the cytokine binds to its receptor, a tyrosine kinase called Jak3 (Janus-kinase 3) is recruited to the receptor at the plasma membrane. This kinase phosphorylates the tyrosine residues of multiple proteins including the [symbol] c-subunit, STAT5 (Signal Transducer and Activator of Transcription 5) and subunits of the PI3 (Phosphatidylinositol 3) kinase. Among these, the phosphorylation of STAT5 has been implicated in many studies as being linked to the proliferation of cells initiated by the [symbol] c-cytokine. (Reviewed in Hennighausen and Robinson, 2008 Genes Dev. 22:711-21.) In accordance with these published data, whether or not the BNZ- [symbol] peptide inhibits the tyrosine phosphorylation of STAT5 molecule in PT-18 [symbol] cells stimulated by IL-15 was examined (results shown in FIG. 4).

PT-18 [symbol] cells were stimulated by IL-15 in the presence or absence of BNZ- [symbol] peptide. Cytoplasmic proteins were extracted from the cells according to a conventional method as described in Tagaya et al. 1996 EMBO J. 15:4928-39. The extracted cytoplasmic proteins were resolved using a standard SDS-PAGE (Sodium Dodecyl-Sulfate PolyAcrylamide Gel Electrophoresis) and the phosphorylation status was confirmed by an anti-phospho-STAT5 antibody (Cell Signaling Technology, Catalog #9354, Danvers MA) using immunoblotting (See FIG. 4, top panel). To confirm that each lane represented a similar total protein load, the membrane was then stripped, and re-probed with an anti-STAT5 antibody (Cell Signaling Technology, Catalog # 9358) (See FIG. 4, bottom panel).

These results demonstrated that tyrosine phosphorylation of STAT5, a marker of signal transduction, was induced by IL-15 in PT-18 [symbol] cells, and tyrosine phosphorylation of STAT5 was markedly reduced by the BNZ- [symbol] peptide.

### Example 6—Rational Design for C-Antagonist Peptide Derivatives

Derivative peptides are prepared based from the core sequence D/E-F-L-E/Q/N-S/R-X-I/K-X-L/I-X-Q (SEQ ID NO: 2) (where X denotes any amino acid) by substituting the defined amino acids of the core sequence with amino acids having identical physico-chemical properties as designated in FIG. 2.

Alternatively, custom peptides or their derivative peptides can be prepared based on the sequence alignment of the D-helix regions of different [symbol] c-cytokine family members.

### Example 7—Method of Identifying the Inhibitory Specificity of Antagonistic Custom Derivative Peptides

The [symbol] c-cytokine inhibitory specificity of antagonistic custom derivative peptides is determined by assaying the ability of a custom derivative peptide to inhibit the proliferative response of a cytokine-responsive cell line to each of the [symbol] c-cytokines. For example, a mouse cell line, CTLL-2, is used to determine if a candidate peptide inhibits the function of IL-2 and IL-15. PT-18( [symbol] ) cells are used to determine if a candidate peptide inhibits the function of IL-4 and IL-9. PT-18 (7 [symbol] ) cells are used to determine if a candidate peptide inhibits the function of IL-7, and PT-18(21 [symbol] ) cells are used to determine if a candidate peptide inhibits the function of IL-21. PT-18( [symbol] ) denotes a subclone of PT-18 cells that exogenously express human IL-2R [symbol] [symbol] by gene transfection (See Tagaya et al. 1996), PT-18(7 [symbol] ) denotes a subclone that expresses human IL-7R [symbol] by gene transfection and PT-18(21R [symbol] ) cells express human IL-21Rρ.

Another alternative is to use other cell lines that respond to an array of cytokines. An example of this cell line in a human NK cell line NK92 that is commercially available by ATCC (catalog #CRL-2407). This cell line is an IL-2 dependent cell line that responds to other cytokines including IL-9, IL-7, IL-15, IL-12, IL-18, IL-21 (Gong et al. 1994 Leukemia 8: 652-658., Kingemann et al., 1996, Biol Blood Marrow Transplant 2:68; 75, Hodge D L et al., 2002 J. Immunol. 168:9090-8).

### Example 8—Preparation of C-Antagonist Peptides

Custom derivative [symbol] c-antagonist peptides are synthesized chemically by manual and automated processes.

Manual synthesis: Classical liquid-phase synthesis is employed, which involves coupling the carboxyl group or [symbol] -terminus of one amino acid to the amino group or N-terminus of another. Alternatively, solid-phase peptide synthesis (SPPS) is utilized.

Automated synthesis: Many commercial companies provide automated peptide synthesis for a cost. These companies use various commercial peptide synthesizers, including synthesizers provided by Applied Biosystems (ABI). Custom derivative [symbol] c-antagonist peptides are synthesized by automated peptide synthesizers.

### Example 9—Biological Production of Custom Derivative C-Antagonist Peptides Using Recombinant Technology

A custom derivative [symbol] c-antagonist peptide is synthesized biologically as a pro-peptide that consists of an appropriate tagging peptide, a signal peptide, or a peptide derived from a known human protein that enhances or stabilizes the structure of the BNZ- [symbol] peptide and improves their biological activities. If desired, an appropriate enzyme-cleavage sequence proceeding to the N-terminus of the peptide shall be designed to remove the tag or any part of the peptide from the final protein.

A nucleotide sequence encoding the custom derivative peptide with a stop codon at the 3′ end is inserted into a commercial vector with a tag portion derived from thioredoxin of E. coli and a special peptide sequence that is recognized and digested by an appropriate proteolytic enzyme (for example, enterokinase) intervening between the tag portion and the nucleotide sequence encoding the custom derivative peptide and stop codon. One example of a suitable vector is the pThioHis plasmid available from Invitrogen, CA. Other expression vectors may be used.

### Example 10—Conjugation of Custom Peptides and Derivative to Carrier Proteins for Immunization Purposes and Generation of Antibody Against the Custom Peptides

BNZ- [symbol] or a derivative thereof are used to immunize animals to obtain polyclonal and monoclonal antibodies. Peptides are conjugated to the N- or the C-terminus of appropriate carrier proteins (for example, bovine serum albumin, Keyhole Limpet Hemocyanin (KLH), etc.) by conventional methods using Glutaraldehyde or m-Maleimidobenzoyl-N-Hydroxysuccinimide Ester. The conjugated peptides in conjunction with an appropriate adjuvant are then used to immunize animals such as rabbits, rodents, or donkeys. The resultant antibodies are examined for specificity using conventional methods. If the resultant antibodies react with the immunogenic peptide, they are then tested for the ability to inhibit individual [symbol] c-cytokine activity according to the cellular proliferation assays described in Examples 1-3. Due to the composite nature of the derivative peptides it is possible to generate a single antibody that recognizes two different cytokines simultaneously, because of the composite nature of these peptides.

### Example 11—Method for Large Scale Production of Custom Derivative C-Antagonist Peptides

Recombinant proteins are produced in large scale by the use of cell-free system as described elsewhere. (See Takai et al., 2010 Curr. Pharm. Biotechnol. 11(3):272-8.) Briefly, cDNAs encoding the γc-antagonist peptide and a tag are subcloned into an appropriate vector (Sec Takai et al., 2010 Curr. Pharm. Biotechnol. 11(3):272-8), which is subjected to in vitro transcription, followed immediately by an in vitro translation to produce the tagged peptide. The pro-polypeptide is then purified using an immobilized antibody recognizing the tagged epitope, treated by the proteolytic enzyme and the eluate (which mostly contains the custom derivative peptide of interest) is tested for purity using conventional 18% Tricine-SDS-PAGE (Invitrogen) and conventional comassie staining. Should the desired purity of the peptide not be met (>98%), the mixture is subjected to conventional HPLC (high-performance liquid chromatography) for further purification.

### Example 12—Use of Humanized NSG Mouse Model for the Therapeutic Investigation of Immune-Mediated Alopecia and Alopecia Associated Disorders

A major advancement for the in vivo study of human immunological systems was the development that a functional human immune system can be established in a severely immunodeficient mouse such as an immunocompromised NOD/Scid/Il2rg^{-/-} (NSG) mouse. (Shultz et al., 2012 Nat. Rev. Immunol. 12:786-98.) NSG mice lack a functioning [symbol] c-subunit required for [symbol] c-cytokine signaling, are extremely deficient in lymphoid cells, and allow for very efficient human immune system engraftment after intraperitoneal administration of Ficoll-gradient purified human peripheral blood mononuclear cells (huPBMCs). The subsequent expansion of human immune cells results in a humanized mouse model of systemic graft versus host disease (GvHD) as the human T cells target murine tissues including the skin (Sonntag et al., 2015 J. Autoimmun. 62:55-66.) The humanized NSG mice develop a progressive hair loss (alopecia) as one symptom of systemic GvHD, with bald patches appearing after about 3-4 weeks, which progress to a complete loss of hair by about day 45-50. Animals die shortly after due to GvHD.

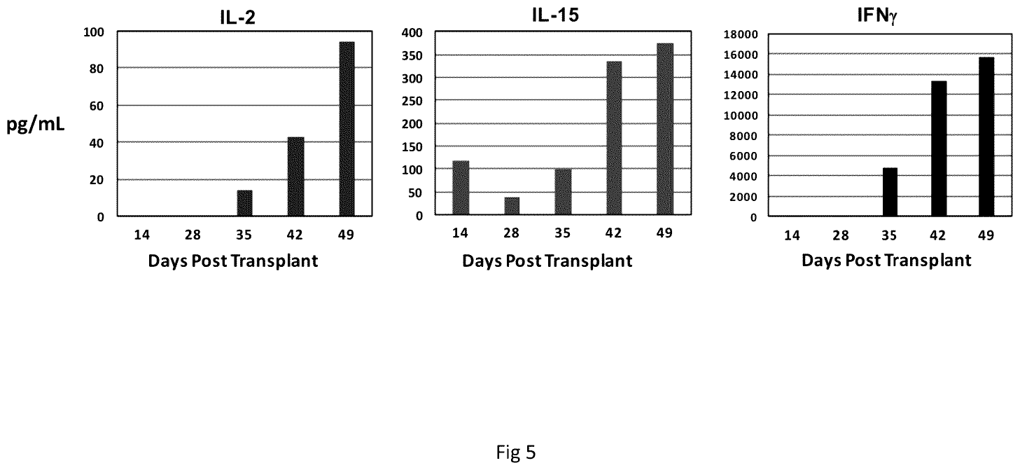

To further understand the mechanisms underlying alopecia in the humanized mouse model, the expression profiles of three key circulating human cytokines (IL-2, IL-15, and IFN [symbol] [symbol] [symbol] were characterized for alopecia following the administration of 2 million huPBMCs intraperitoneally into five 3-week-old NSG mice. Increases in IL-15 were earliest and evident at day 14, while IL-2 and IFN [symbol] [symbol] were not elevated until day 35, with all three cytokines increasing out to day 49 (results shown in FIG. 5), which was the last time point available due to the death of the mice in the experimental group. This indicates that IL-15 is a key driver of disease. By day 35, mice showed symptoms of GvH responses including loss of body weight and moderate to severe alopecia.

### Example 13—Effects of an Anti-Human CD8 Antibody on Humanized NSG Mice with Immune-Mediated Hair Loss

Members of the NKG2 family have been implicated in the cytotoxicity process of NK and CD8+ T cells and are regulated by multiple cytokines including the [symbol] c-cytokine IL-15 (Borrego et al. 1998 J Exp Med 187:813-18, Brumbaugh et al. 1996 J Immunol 157:2804-12, Cantoni et al. 1998 Eur J Immunol 28:327-38, Mingari et al. 1998 Proc Natl Acad Sci 95:1172-7). Each NKG2 receptor dimerizes with the lectin protein CD94 to form a heterodimeric receptor complex (Lazetic et al. 1996 J Immunol 157:4741-5), except NKG2D which exists as a homodimer (Garrity et al. 2005 Proc Natl Acad Sci 102:7641-6). Previous reports suggest that hair loss in patients with alopecia is mediated by cytotoxic CD8+ T-cells that express the NKG2D receptor (Xing et al. 2014 Nat Med 9:1043-9., Gilhar et al. 2016 Autoimmun. Rev. 15:726-35.) To characterize the importance of CD8+ T-cells in this disease model, animals were treated with the anti-human CD8 antibody (OKT8) (BD Biosciences), which depletes human CD8+ T-cells. Within 4 weeks after transplantation of 2 million huPBMCs, a cohort of five mice developed weight loss and patchy to complete hair loss. Three humanized mice were then selected for treatment with two injections (twice/week) of 50 μg/mouse of the anti-CD8 antibody.

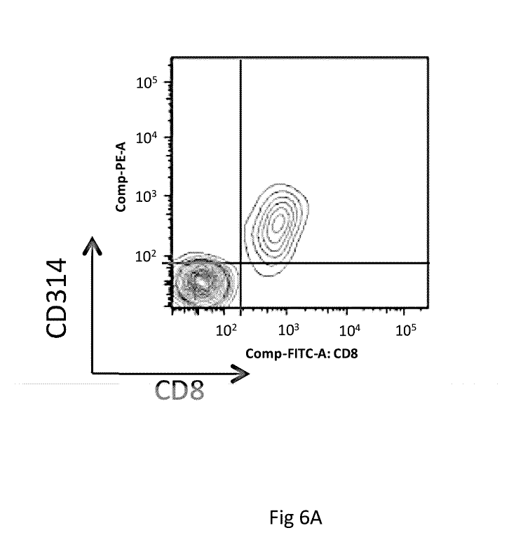

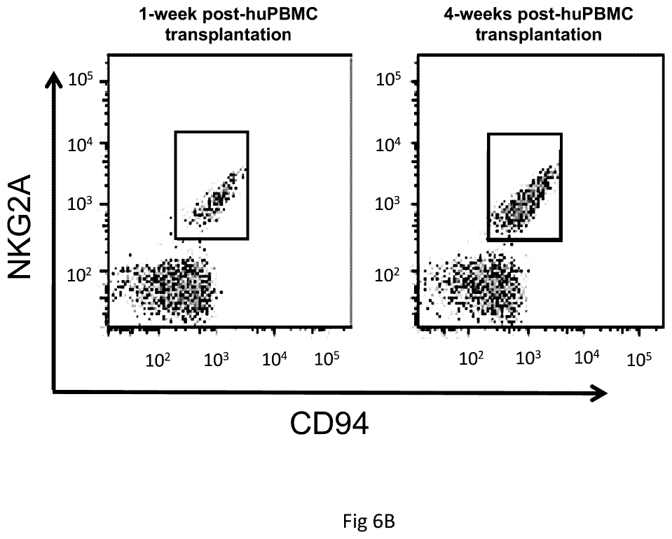

Prior to treatment with the anti-human CD8 antibody, human CD8+ T cells were isolated from a blood sample collected from a representative humanized NSG mouse, and stained for the expression of the NKG2D (CD314) receptor, and receptors in the NKG2 family (NKG2A and NKG2C) to facilitate measurement by flow cytometry. The cytotoxic CD8+ T cells in alopecia disease progression have also been characterized as positive for the expression of the activating NKG2D receptor (Xing et al. 2014 Nat Med 9:1043-9.) Flow cytometry showed that almost the entire human CD8+ T-cell population isolated from the humanized NSG mouse was NKG2D+ (scc FIG. 6A). Interestingly, whereas it was observed that the human NKG2C+ CD8+ T-cells diminish after huPBMC transplantation, the human NKG2A+ CD8+ T-cells showed a marked increase after huPBMC transplantation that only expanded as GvHD symptoms worsened and the disease progressed (See FIG. 6B).

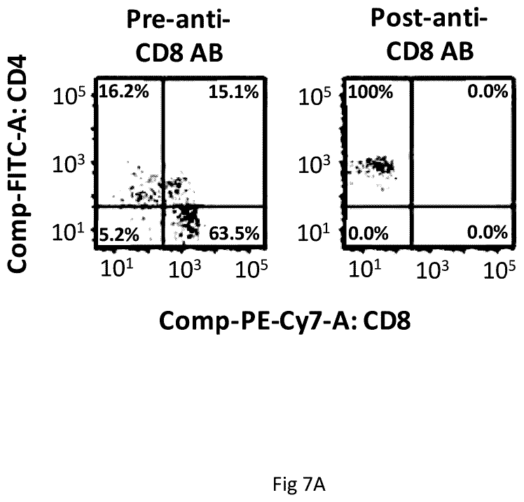

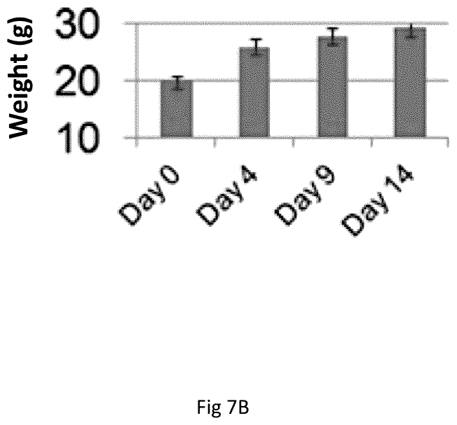

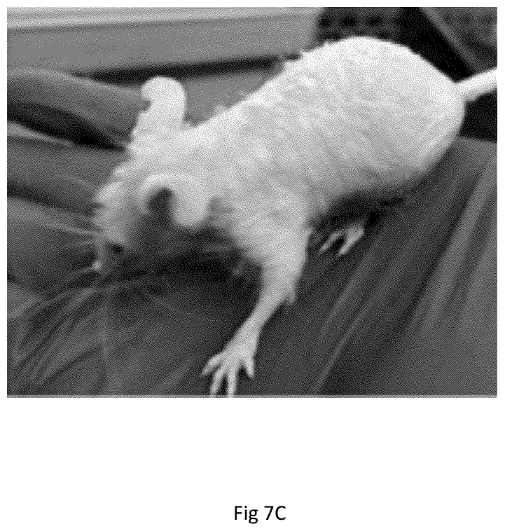

Following treatment with the anti-human CD8 antibody, all human CD8+ T-cells were significantly and specifically depleted (See FIG. 7A), which did not re-emerge post treatment. Within 4 days-post depletion of CD8+ T-cells, all three humanized mice showed weight gain (See FIG. 7B), with re-growth of body hair evident by two weeks-post treatment (see FIG. 7C).

### Example 14—Constitutive C-Signaling of Human NKG2A+ CD8+ T-Cells in Humanized NSG Mice with Immune-Mediated Hair Loss

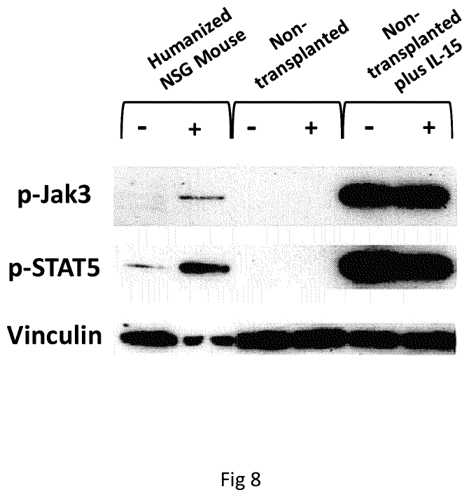

The interaction between the [symbol] c-subunit and a [symbol] c-cytokine leads to the activation and phosphorylation of Jak3. Considering the interaction of the [symbol] c-subunit and Jak3 is very specific in that there is no other receptor molecule that recruits Jak3 for signal transduction, it was next tested whether human NKG2A+ CD8+ T-cells isolated from humanized NSG mice 4 weeks after transplantation of 2 million huPBMCs were positive for the phosphorylation of Jak3 and the downstream phosphorylation of STAT5. Human NKG2A+ and NKG2A−CD8+ T-cells were Ficoll-purified from blood and spleen of three representative humanized NSG mice. Cells were then stained by a mixture of FITC-anti-CD4, PE-anti-CD8, and PE/Cy7-anti-NKG2A, and fluorescence-activated cell sorted (FACSAria II, BD Biosciences) into CD4−CD8+ NKG2A+ and CD4−CD8+ NKG2A− subpopulations. As a control, non-transplanted NKG2A+ and NKG2A−CD8+ T-cells were left unstimulated, or stimulated by the addition of IL-15 ex vivo. Cytoplasmic proteins were extracted from the cells according to a conventional method as described in Tagaya et al. 1996 EMBO J. 15:4928-39. The extracted cytoplasmic proteins were resolved using a standard SDS-PAGE (Sodium Dodecyl-Sulfate PolyAcrylamide Gel Electrophoresis) and the phosphorylation status was confirmed by an anti-phospho-Jak3 antibody (Cell Signaling Technology, Catalog #5031, Danvers MA) or an anti-phospho-STAT5 antibody (Cell Signaling Technology, Catalog #9354, Danvers MA) using immunoblotting (see FIG. 8). Vinculin was probed as a control. Results show constitutive [symbol] c-signaling of human NKG2A+, but not NKG2A−CD8+ T-cells in humanized NSG mice 4 weeks after transplantation of 2 million huPBMCs.

### Example 15—Antibody-Mediated Depletion of Members of the Human C-Type Lectin Receptor NKG2 Family in CD8+ T-cells on Humanized NSG Mice with Immune-Mediated Hair Loss

To test the causative involvement of members of the human C-type lectin receptor NKG2 family (NKG2A, B, C, D, E, F, and H) in CD8+ T-cells in the pathogenesis of systemic GvHD in the humanized NSG mouse, antibody-mediated depletion of each individual human NKG2 protein member in CD8+ T-cells is performed by injecting 50 μg/mouse of the anti-NKG2 antibody specific to the NKG2 protein member under study twice per week in three representative humanized NSG mice at 3-to 5-wecks post-transplantation of 2 million huPBMCs. The successful depletion of the specific NKG2 family member in CD8+ T-cells is then correlated with major systemic GvHD symptoms, such as loss of body weight, immune-mediated hair loss, and circulating levels of the cytokines IL-2, IL-15, and IFN [symbol] .

### Example 16—Effects of Antibody-Mediated Depletion of Human NKG2A+ CD8+ T-Cells on Humanized NSG Mice with Immune-Mediated Hair Loss

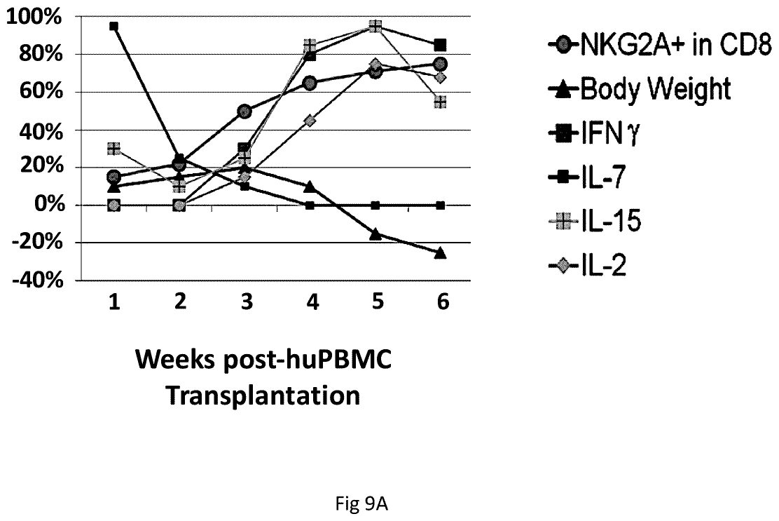

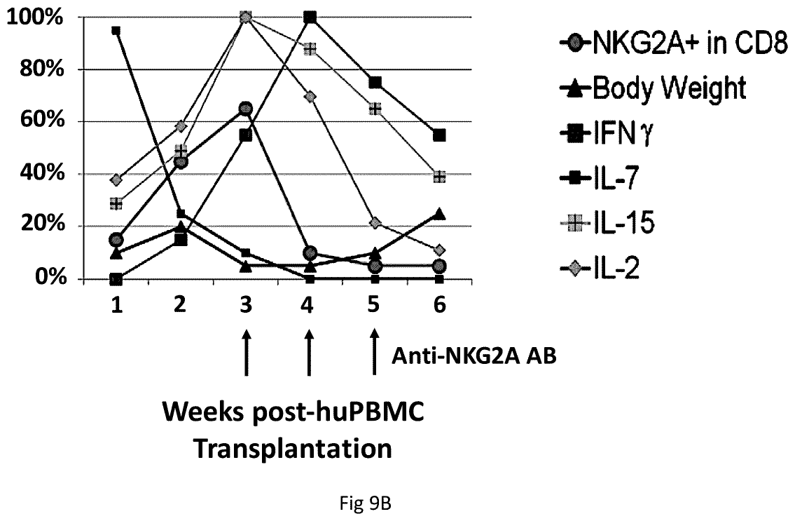

To further examine if NKG2A+ CD8+ T-cells are causatively linked to the systemic GvHD symptoms such as loss of body weight and hair loss observed following huPBMC transplantation in NSG mice, a compilation was generated of the kinetics of the expansion of NKG2A+ CD8+ T-cells with those of body weight and the levels of the inflammatory cytokine IFN [symbol] [symbol] and the [symbol] c-cytokines IL-2, IL-7, and IL-15 weekly from three representative humanized NSG mice 1-weck to 6-weeks post-transplantation of 2 million huPBMCs. A clear correlation was observed between the increase of NKG2A+ cells in the CD8+ T-cell compartment with an increase of IL-2, IL-15, and IFN [symbol] [symbol] [symbol] see FIG. 9A).

To test the causative involvement of NKG2A+ CD8+ T-cells in the pathogenesis of systemic GvHD in the humanized NSG mouse, antibody-mediated depletion of human NKG2A+ CD8+ T-cells was performed by injecting 50 μg/mouse of an anti-NKG2A antibody (R & D Systems, Catalog #MAB1059, Clone 131411, Minneapolis, MN) twice per week in three representative humanized NSG mice at 3- to 5-weeks post-transplantation of 2 million huPBMCs. The successful depletion of NKG2A+ CD8+ T-cells (See FIG. 9B, weeks 4-6 post-huPBMC transplantation) was positively associated with the mitigation of major systemic GvHD symptoms, with the loss of body weight and immune-mediated hair loss improving after the first week of anti-NKG2A antibody treatment. It was observed that a decrease of IL-2, IL-15, and IFN [symbol] [symbol] directly correlated with the antibody-mediated depletion of human NKG2A+ CD8+ T-cells (see FIG. 9B).

### Example 17—Effects of BNZ- on Humanized NSG Mice with Immune-Mediated Hair Loss

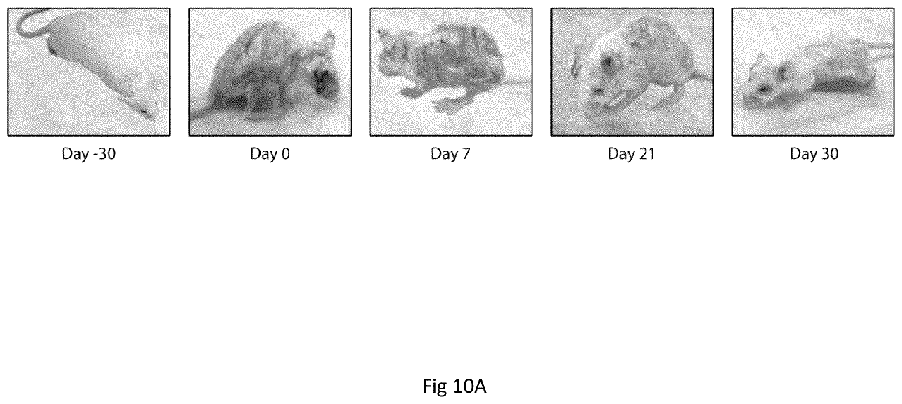

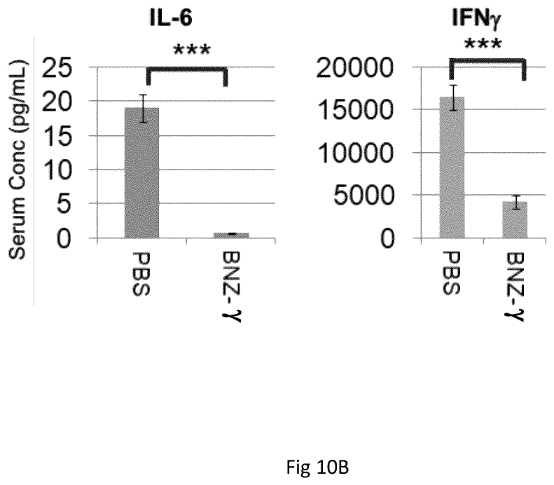

To test the effects of BNZ- [symbol] [symbol] [symbol] five humanized NSG mice were allowed to develop extensive GvHD with widespread hair loss prior to initiating treatment (approximately 4-weeks post 2 million huPBMC transplant). At the start of twice weekly intravenous (IV) treatment with a PEGylated BNZ- [symbol] (Day 0, 2 mg/kg) for 2 weeks, all animals appeared very sick. Control PBS-treated animals died within approximately 1-2 weeks. By day 21, BNZ- [symbol] -treated animals gained significant weight, had healthier-looking skin, and visible regrowth of their fur coat. The effect of BNZ- [symbol] continued ˜2 weeks after completing the two-week treatment, with the BNZ- [symbol] -treated animals showing significant regrowth of their fur (results shown in FIG. 10A). In support of the clinical observations, BNZ- [symbol] resulted in a statistically significant reduction in the levels of circulating inflammatory cytokines (IL-6 and IFN [symbol] [symbol] , back to/towards the normal physiological range in the NSG mouse following completion of the twice weekly BNZ-γ dosing regimen for a treatment duration of two weeks (see FIG. 10B).

### Example 18—Comparison of BNZ-, Anti-IL-2 Antibody, Anti-IL-15 Antibody, and Combination Anti-IL-2 and Anti-IL-15 Antibody Treatment on Survival, Immune-Mediated Hair Loss and Cytokine Levels in Humanized NSG Mice

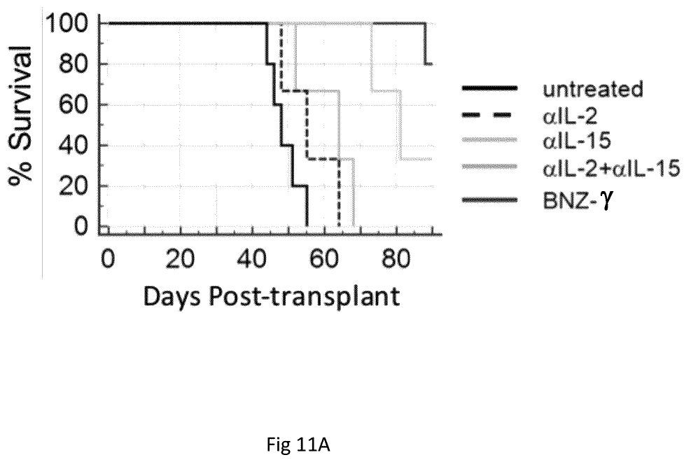

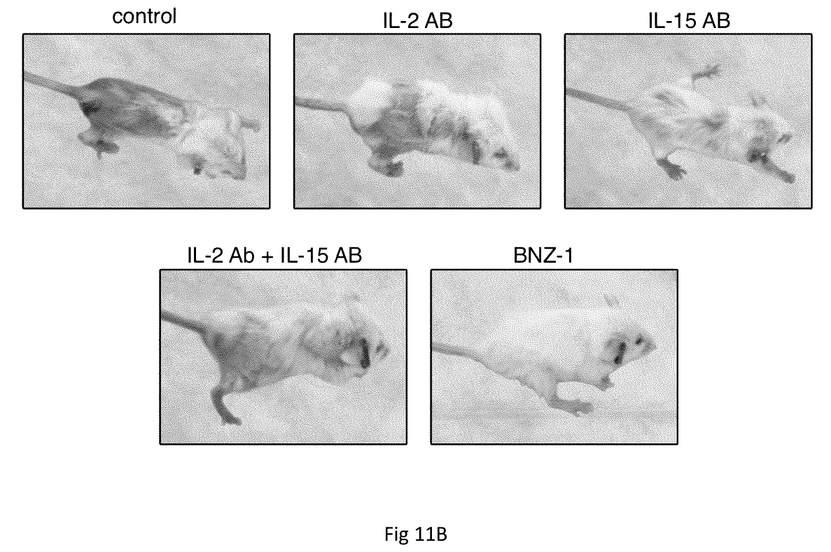

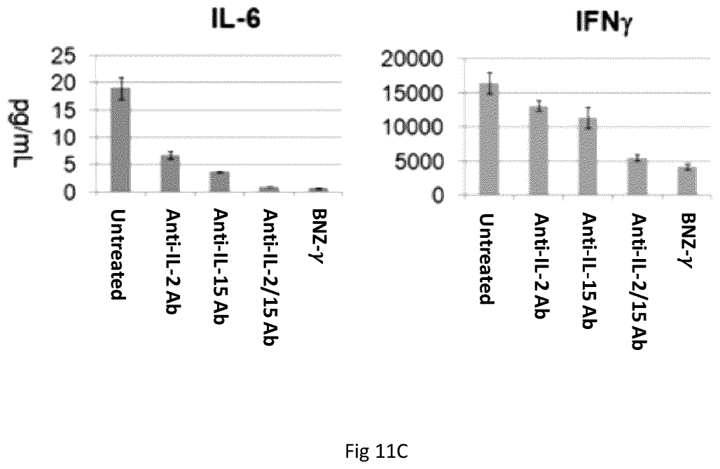

In this experiment, NSG mice were transplanted with 2 million huPBMCs on study day 0, with therapeutic treatment beginning 35-days post-transplant. Mice were treated twice weekly with IV injections of PBS control (n=5), BNZ- [symbol] at 2 mg/kg (n=5), anti-IL-2 antibody at 5 mg/kg (n=3), anti-IL-15 antibody at 5 mg/kg (n=3), or combination anti-IL-2 and anti-IL-15 antibody each at 5 mg/kg (n=3) starting on day 35 for a treatment duration of 4 weeks. PBS control mice began dying shortly after treatment initiation, while the single antibody-treated animals began to die after treatment was stopped, which was not statistically different from untreated controls (p>0.05). The combination of anti-IL-2 and anti-IL-15 antibodies was significantly more effective as compared to single antibody treatment (p=0.014) with a survival benefit that lasted several weeks after cessation of treatment, but was less effective than BNZ- [symbol] (p=0.001) (results shown in FIG. 11A).

At the beginning of treatment on post-transplant day 35, mice had significant hair loss. Approximately two weeks after treatment was completed (˜day 63), there was a noticeable improvement in the regrowth of hair in animals treated with the anti-IL-15 antibody, which appeared to be more effective as compared to the anti-IL-2 antibody. The combination antibody treatment did not appear significantly different for hair regrowth as compared to the anti-IL-15 antibody alone. However, the BNZ- [symbol] -treated mice appeared to have the greatest degree of hair regrowth of all 4 treatment groups, which suggests that blockade of IL-9 may be important to achieve the maximum therapeutic response. (results shown in FIG. 11B).

Levels of IL-6 and IFN [symbol] were also measured in this experiment. Both inflammatory cytokines showed significant elevations in the PBS control NSG mice. All 4 active treatments reduced the levels of each cytokine to varying degrees, with BNZ- [symbol] and the combination antibody being most effective. These data are consistent with previous reports that IFN [symbol] [symbol] is a downstream cytokine regulated by IL-15, with IL-15 blockade shutting down IFN [symbol] expression (Fehniger et al. 2000 J. Immunol. 164:1643-7). Cytokine levels were determined using sera collected on day 50, except for one animal in the anti-IL-2 antibody treatment group (collected on day 45), one mouse in the PBS control untreated group (collected on day 45), and two mice from the PBS control untreated group (collected on day 40) to ensure a blood sample was collected before each became fatally ill. (results shown in FIG. 11C).

### Example 19—Immunohistochemistry of Humanized NSG Mouse Skin Tissue Treated with BNZ-

In order to characterize the nature of immune attack in the skin tissue and around the hair follicles, immunohistochemistry studies were conducted of the skin tissue of humanized NSG mice 3-weeks (pre-BNZ- [symbol] ) and 7-weeks (with or without BNZ- [symbol] [symbol] treatment) after transplantation of 2 million huPBMCs. The tissue was fixed for 24 hours in 4% formalin (Sigma) and then moved to 70% ethanol for at least 24 hours before being processed. Tissue was then embedded in paraffin following dehydration for two washes of two hours each in 70%, 90%, and 100% ethanol, then cleared in xylene twice for two hours each, and infiltrated with melted paraplast plus at 60C two times for two hours. Paraffin embedded tissues were stored at room temperature prior to sectioning and staining. An anti-human CD8 antibody (BioCare Medical CRM 311C) or isotype control was used for staining of the tissues based on the standard procedure for IHC.

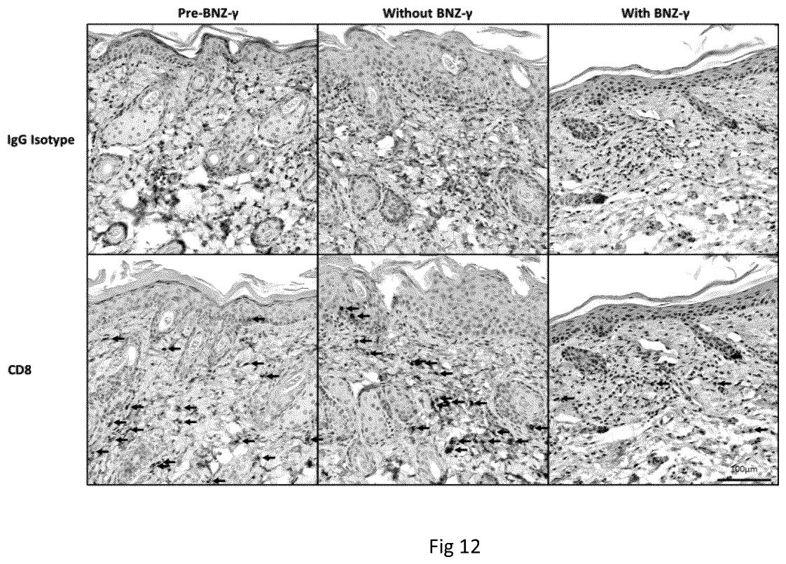

An influx of human CD8 T cells in the skin tissue of humanized NSG mice at 3-weeks post-transplant was observed. CD8 T cells remained at comparable levels at 7-weeks post-transplant without BNZ- [symbol] [symbol] treatment. However, at 7-weeks post-transplant with BNZ- [symbol] treatment, a significant reduction in the number of infiltrated CD8 cells was observed. The data are shown in FIG. 12.

### Example 20—Method of Treating Alopecia in a Human Patient by Administration of a Therapeutic Compound

A human patient suffering from alopecia (alopecia areata, alopecia totalis, alopecia subtotalis, alopecia universalis, alopecia diffusa, ophiasis-type alopecia areata) is identified. An effective dose, as determined by the physician, of a therapeutic compound, for example, an anti-CD8 antibody, anti-IL-2 antibody, anti-IL-15 antibody, anti-NKG2A antibody, a custom derivative [symbol] -antagonist peptide, for example, a composite peptide comprising the sequence of BNZ- [symbol] [symbol] or a derivative thereof, or a combination of said therapeutic compounds is administered to the patient for a period of time determined by the physician. Treatment is determined to be effective if patient's symptoms improve or if the progression of the disease has been stopped or slowed down.

### Example 21—Method of Treating Vitiligo in a Human Patient by Administration of a Therapeutic Compound

A human patient suffering from vitiligo (vitiligo and rosacea vitiligo) is identified. An effective dose, as determined by the physician, of a therapeutic compound, for example, an anti-CD8 antibody, anti-IL-2 antibody, anti-IL-15 antibody, anti-NKG2A antibody, a custom derivative [symbol] c-antagonist peptide, for example, a composite peptide comprising the sequence of BNZ- [symbol] [symbol] or a derivative thereof, or a combination of said therapeutic compounds is administered to the patient for a period of time determined by the physician. Treatment is determined to be effective if patient's symptoms improve or if the progression of the disease has been stopped or slowed down.

### Example 22—Method of Treating Psoriasis in a Human Patient by Administration of a Therapeutic Compound

A human patient suffering from psoriasis (psoriasis, psoriasis vugaris, psoriasis capitis, psoriasis guttate, psoriasis inversa, psoriatic arthritis) is identified. An effective dose, as determined by the physician, of a therapeutic compound, for example, an anti-CD8 antibody, anti-IL-2 antibody, anti-IL-15 antibody, anti-NKG2A antibody, a custom derivative [symbol] c-antagonist peptide, for example, a composite peptide comprising the sequence of BNZ- [symbol] [symbol] or a derivative thereof, or a combination of said therapeutic compounds is administered to the patient for a period of time determined by the physician. Treatment is determined to be effective if patient's symptoms improve or if the progression of the disease has been stopped or slowed down.

### Example 23—Method of Treating Pemphigus in a Human Patient by Administration of a Therapeutic Compound

A human patient suffering from pemphigus (pemphigus, pemphigus vulgaris, pemphigus foliaceus, pemphigus vegetans, pemphigus erythematosus) is identified. An effective dose, as determined by the physician, of a therapeutic compound, for example, an anti-CD8 antibody, anti-IL-2 antibody, anti-IL-15 antibody, anti-NKG2A antibody, a custom derivative [symbol] c-antagonist peptide, for example, a composite peptide comprising the sequence of BNZ- [symbol] [symbol] or a derivative thereof, or a combination of said therapeutic compounds is administered to the patient for a period of time determined by the physician. Treatment is determined to be effective if patient's symptoms improve or if the progression of the disease has been stopped or slowed down.

### Example 24—Method of Treating Pemphigoid in a Human Patient by Administration of a Therapeutic Compound

A human patient suffering from pemphigoid (mucous membrane pemphigoid, scarring mucous membrane pemphigoid, bullous pemphigoid) is identified. An effective dose, as determined by the physician, of a therapeutic compound, for example, an anti-CD8 antibody, anti-IL-2 antibody, anti-IL-15 antibody, anti-NKG2A antibody, a custom derivative [symbol] c-antagonist peptide, for example, a composite peptide comprising the sequence of BNZ- [symbol] [symbol] or a derivative thereof, or a combination of said therapeutic compounds is administered to the patient for a period of time determined by the physician. Treatment is determined to be effective if patient's symptoms improve or if the progression of the disease has been stopped or slowed down.

### Example 25—Method of Treating GvHD in a Human Patient by Administration of a Therapeutic Compound

A human patient suffering from GvHD is identified. An effective dose, as determined by the physician, of a therapeutic compound, for example, an anti-CD8 antibody, anti-IL-2 antibody, anti-IL-15 antibody, anti-NKG2A antibody, a custom derivative [symbol] c-antagonist peptide, for example, a composite peptide comprising the sequence of BNZ- [symbol] [symbol] or a derivative thereof, or a combination of said therapeutic compounds is administered to the patient for a period of time determined by the physician. Treatment is determined to be effective if patient's symptoms improve or if the progression of the disease has been stopped or slowed down.

### REFERENCES

All references disclosed herein as well as listed below are incorporated by reference in their entireties.

Antony, P. A., Paulos, C. M., Ahmadzadeh, M., Akpinarli, A., Palmer, D. C., Sato, N., Kaiser A., Heinrichs, C. S., Klebanoff, C. A., Tagaya, Y., and Restifo, N P., Interleukin-2-dependent mechanisms of tolerance and immunity in vivo. 2006 J. Immunol. 176:5255-66.

Atwa M. A., Youssef N., Bayoumy N. M. T-helper cytokines (interleukins 17, 21, 22, and 6, and tumor necrosis factor-a) in patients with alopecia areata: association with clinical type and severity. 2016 Int J Dermatol 55:666-72.

Awwad, S. and Angkawinitwong, U., Overview of Antibody Drug Delivery. 2018 Pharmaceutics 10:83.

Azimi, N., Nagai, M., Jacobson, S., Waldmann, T. A., IL-15 plays a major role in the persistence of Tax-specific CD8 cells in HAM/TSP patients. 2001 Proc. Natl. Acad. Sci. 98:14559-64.

Azimi, N., Mariner J., Jacobson S., Waldmann T. A., How does interleukin 15 contribute to the pathogenesis of HTLV type-1 associated myelopathy/tropical spastic paraparesis? 2000 AIDS Res. Hum. Retroviruses 16:1717-22.

Azimi, N., Jacobson, S., Leist, T., Waldmann, T. A., Involvement of IL-15 in the pathogenesis of human T lymphotropic virus type-I-associated myelopathy/tropical spastic paraparesis: implications for therapy with a monoclonal antibody directed to the IL-2/15R beta receptor. 1999 J. Immunol. 163:4064-72.

Azimi, N., Brown, K., Bamford, R. N., Tagaya, Y., Siebenlist, U., Waldmann, T. A., Human T cell lymphotropic virus type I Tax protein trans-activates interleukin 15 gene transcription through an NF-kappaB site. 1998 Proc. Natl. Acad. Sci. USA 95:2452-7.

Bazan, J. F., Hematopoietic receptors and helical cytokines. 1990 Immunol. Today 11:350-354.

Bettini, M., and Vignali, D. A., Regulatory T cells and inhibitory cytokines in autoimmunity. 2009 Curr. Opin. Immunol. 21:612-8.

Blaser, B. W., Roychowdhury, S, Kim, D. J., Schwind, N. R., Bhatt, D., Yuan, W., Kusewitt, D. F., Ferketich, A. K., Caligiuri, M. A., Guimond, M., Donor-derived IL-15 is critical for acute allogeneic graft-versus-host disease. 2005 Blood 105:894-901.

Blažek, D. and Celer, V. The production and application of single-chain antibody fragments. 2003 Folia Microbiol 48:687-98.

Bodd, M., Raki, M., Tollefsen, S., Fallang, L. E., Bergseng, E., Lundin, K. E., Sollid, L. M., HLA-DQ2-restricted gluten-reactive T cells produce IL-21 but not IL-17 or IL-22. 2010 Mucosal Immunol. 3:594-601.

Borrego, F. Ulbrecht, M., Weiss, E. H., Coligan, J. E., Brooks, A. G., Recognition of human histocompatibility leukocyte antigen (HLA)-E complexed with HLA class I signal sequence-derived peptides by CD94/NKG2 confers protection from natural killer cell-mediated lysis. 1998 J Exp Med 187:813-18.

Botti, E., Spallone, G., Caruso, R. Monteleone, G., Chimenti, S., Costanzo, A. Psoriasis, from pathogenesis to therapeutic strategies: IL-21 as a novel potential thereapeutic target. 2012 Curr. Pharm. Biotechnol. 13:1861-1867.

Brumbaugh, K. M., Perez-Villar, J. J., Dick, C. J., Schoon, R. A., Lopez-Botet, M., Leibson, P. J. Clonotypic differences in signaling from CD94 (kp43) on NK cells lead to divergent cellular responses. 1996 J Immunol 157:2804-12.

Bucher, C., Koch, L., Vogtenhuber, C., Goren, E., Munger, M., Panoskaltsis-Mortari, A., Sivakumar, P., Blazar, B. R. IL-21 blockade reduces graft-versus-host disease mortality by supporting inducible T regulatory cell generation. 2009 114:5375-84.

Cantoni, C., Biassoni, R., Pende, D. et al., The activating form of CD94 receptor complex: CD94 covalently associates with the Kp39 protein that represents the product of the NKG2-C gene. 1998 Eur J Immunol 28:327-38.

Caruso, R., Costanzo, A., Monteleone, G. Pathogenic role of interleukin-21 in psoriasis. 2009 Cell Cycle 8:3629-3630.

Caruso, R., Bott, E., Sarra, M., Esposito, M., Stolfi, C., Diluvio, L., Giustizieri, M. L., Pacciani, V., Mazzotta, A., Campione, E. et al. Involvement of interleukin-21 in the epidermal hyperplasia of psoriasis. 2009 Nat. Med. 15:1013-1015.

Chik, K. W., Li, K., Pong, H., Shing, M. M., Li, C. K., Yuen, P. M. Elevated serum interleukin-15 level in acute graft-versus-host disease after hematopoietic cell transplantation. 2003 J Pediatr Hematol Oncol. 25:960-4.

Cox, K. L., Devanarayan, V., Kriauciunas, A., Montrose, C., and Sittampalam, S. “Immunoassay methods”, in Assay Guidance Manual [Internet], 2004 eds G.S. Sittampalam, N. P. Coussens, H. Nelson, et al. (Bethesda, MD: Eli Lilly & Company and the National Center for Advancing Translational Sciences).

D'Auria, L., Bonifati, C., Cordiali-Fei, P., Leone, G., Picardo, M., Pietravalle, M., Giacalone, B., Ameglio, F., Increased serum interleukin-15 levels in bullous skin diseases: correlation with disease intensity. 1999 Arch. Dermatol. Res. 291:354-356.

De Rezende, L. C., Silva I. V., Rangel, L. B., Guimaraes, M. C., Regulatory T cells as a target for cancer therapy. 2010 Arch. Immunol. Ther. Exp. 58:179-90.

Dubois, S., Mariner, J., Waldmann, T. A., Tagaya, Y., IL-15Ralpha recycles and presents IL-15 In trans to neighboring cells. 2002 Immunity 17:537-47.

Dodge D L. Et al., IL-2 and IL-12 alter NK cell responsiveness to IFN-gamma-inducible protein 10 by down-regulating CXCR3 expression.J. Immun. 168:6090-8.

Fang J., Hu, C., Hong, M., Wu, Q., You, Y., Zhong, Z., Li, W., Zou, P., Hu, Y., Prophylactic effects of interleukin-2 receptor antagonists against graft-versus-host disease following unrelated donor peripheral blood stem cell transplantation. 2012 Biol Blood Marrow Transplant. 18:754-62.

Fehniger, T. A., Yu, H., Cooper, M. A., Suzuki, K., Shah, M. H., Caligiuri, M. A. IL-15 costimulates the generalized Shwartzman reaction and innate IFN-gamma production in vivo. 2000 J. Immunol. 164:1643-1647.

Fehniger, T. A., Suzuki, K., Ponnappan, A., VanDeusen, J. B., Cooper, M. A., Florea, S. M., Freud, A.G., Robinson, M. L., Durbin, J., Caligiuri, M. A., Fatal leukemia in interleukin 15 transgenic mice follows early expansions in natural killer and memory phenotype CD8+ T cells. 2001 J. Exp. Med. 193:219-31.

Fisher, A. G., Burdet, C., LeMeur, M., Haasner, D., Gerber, P., Cerediq, R., Lymphoproliferative disorders in an IL-7 transgenic mouse line. 1993 Leukemia 2:S66-68.

Frenzel, A., Kügler, J., Helmsing, S., Meier, D., Schirrmann, T., Hust, M., and Dübel, S. Designing Human Antibodies by Phage Display. 2017 Transfus Med Hemother 44:312-18.

Fuentes-Duculan, J., Gulati, N., Bonifacio, K. M., Kunjravia, N., Zheng, X., Suarez-Farinas, M., Shemer, A., Guttman-Yassky, E., Krueger, J. G., Biomarkers of alopecia areata disease activity and response to corticosteroid treatment. 2016 Exp Dermatol 4:282-6.

Garrity, D., Call, M. E., Feng, J., Wucherpfennig, K. W., The activating NKG2D receptor assembles in the membrane with two signaling dimers into a hexameric structure. 2005 Proc Natl Acad Sci 102:7641-6.

Gilhar, A., Schrum, A. G., Etzioni, A., Waldmann, H., Paus, R. Alopecia areata animal models illuminate autoimmune pathogenesis and novel immunotherapeutic strategies. 2016 Autoimmun. Rev. 15:726-735.

Gong J. H. et al. Characterization of a human cell line (NK-92) with phenotypical and functional characteristics of activated natural killer cells. Leukemia 8: 652-658, 1994.

Grando S. A., Glukhenky, B. T., Drannik, G. N., Epshtein, E. V., Kostromin, A. P., Korostash, T. A., Mediators of inflammation in blister fluids from patients with pemphigus vulgaris and bullous pemphigoid. 1989 Arch. Dermatol. 125:925-930.

Guo-Qiang, B., and Xian-Li, H. Guided selection methods through chain shuffling. 2009 Methods Mol Biol 562:133-42.

Hammers, C. M. and Stanley, J. R. Antibody Phage Display: Technique and Applications. 2014 J Invest Dermatol 134:e17.

He, Z., Jin, L., Liu, Z. F., Hu., L., Dang, E. L., Feng, Z. Z., Li, Q. J., Wang, G. Elevated serum levels of interleukin 21 are associated with disease severity in patients with psoriasis. 2012 Br. J. Dermatol. 167:191-193.

Hennighausen, L., Robinson, G.W., Interpretation of cytokine signaling through the transcription factors STAT5A and STAT5B. 2008 Genes Dev. 22:711-21.

Hippen, K. L., Bucher, C., Schirm, D. K., Bearl, A. M., Brender, T., Mink, K. A., Waggie, K. S., Peffault de Latour, R., Janin, A., Curtsinger, J. M. et al. Blocking IL-21 signaling ameliorates xenogeneic GVHD induced by human lymphocytes. 2012 Blood 119:619-28.

Jagielska D., Redler S., Brockschmidt F. F., Herold C., Pasternack S. M., Garcia Bartels N., Hanneken S., Eigelshoven S., Refke M., Barth S., et al. Follow-up study of the first genome-wide association scan in alopecia areata: IL13 and KIAA0350 as susceptibility loci supported with genome-wide significance. 2012 J Invest Dermatol 132:2192-7.

Jespers, L. S., Roberts, A., Mahler, S. M., Winter, G., and Hoogenboom, H. R. Guiding the selection of human antibodies from phage display repertoires to a single epitope of an antigen. 1994 Biotechnology 12:899-903.

Jespersen, M. C., Peters, B., Nielsen, M., and Marcatili, P. BepiPred-2.0: improving sequence-based B-cell epitope prediction using conformational epitopes. 2017 Nucleic Acids Res 45:W24-W29.

Klingemann H G, et al. A cytotoxic NK-cell line (NK-92) for ex vivo purging of leukemia from blood. Biol. Blood Marrow Transplant. 2: 68-75, 1996.

Köhler, G. and Milstein, C. Continuous cultures of fused cells secreting antibody of predefined specificity. 1975 Nature 256:495-7.

Kooy-Winkelaar, Y. M, Bouwer, D., Janssen, G. M., Thompson, A., Brugman, M. H., Schmitz, F., de Ru, A. H., van Gils, T., Bouma, G., van Rood, J. J. et al. CD4 T-cell cytokines synergize to induce proliferation of malignant and nonmalignant intraepithelial lymphocytes. 2017 Proc Natl Acad Sci USA 114: E980-9.

Kozbor, D. and Roder, J.C. The production of monoclonal antibodies from human lymphocytes. 1983 Immunol Today 4:72-9.

Kozbor, D., Lagarde, A., and Roder, J. C. Human hybridomas constructed with antigen-specific, EBV-transformed cell lines. 1982 Proc Natl Acad Sci 79:6651-55.

Krause, C. D. and Pestka, S., Evolution of the Class 2 cytokines and receptors, and discovery of new friends and relatives. 2005 Pharmacol. and Therapeutics 106:299-346.

Kundig, T. M., Schorle, H., Bachmann, M. F., Hengartener, H., Zinkernagel, R. M., Horak, I., Immune Responses of the interleukin-2-deficient mice. 1993 Science 262:1059-61.

Laffleur, B., Pascal, V., Sirac, C., and Cogné, M. Production of human or humanized antibodies in mice. 2012 Methods Mol Biol 901:149-59.

Lazetic, S., Chang, C., Houchins, J. P., Lanier, L. L., Phillips, J. H., Human natural killer cell receptors involved in MHC class I recognition are disulfide-linked heterodimers of CD94 and NKG2 subunits. 1996 J Immunol 157:4741-5.

Le Buanec, H., Paturance, S., Couillin, I., Schnyder-Candrian, S., Larcier, P., Ryffel, B., Bizzini, B., Bensussan, A., Burny, A., Gallo, R., Zagury, D., Peltre, G., Control of allergic reactions in mice by an active anti-murine IL-4 immunization. 2007 Vaccine 25:7206-16.

Littman, D. R., Rudensky, AY., Th17 and regulatory T cells in mediating and restraining inflammation. 2010 Cell 140(6):845-58.

Lonberg, N. and Huszar, D. Human antibodies from transgenic mice. 1995 Int Rev Immunol 13:65-93.

Mingari, M.C., Ponte, M., Bertone, S. et al. HLA class I-specific inhibitory receptors in human T lymphocytes: interleukin 15-induced expression of CD94/NK62A in superantigen-or alloantigen-activated CD8+ T cells. 1998 Proc Natl Acad Sci 95:1172-7.

Miyagawa, F., Tagaya, Y., Kim, B. S., Patel, H. J., Ishida, K., Ohteki, T., Waldmann, T.A., Katz, S.I., IL-15 serves as a costimulator in determining the activity of autoreactive CD8 T cells in an experimental mouse model of graft-versus-host-like disease. 2008 J. Immunol. 181:1109-19.

Noguchi, M., Yi, H., Rosenblatt, H. M., Filipovich, A. H., Adelstein, S., Modi, W. S., McBride, O. W., Leonard, W. J., Interleukin 2 receptor gamma chain mutation results in X-linked severe combined immunodeficiency in humans. 1993 Cell 73:147-157.

O H, U., Jacobson S., Treatment of HTLV-I-Associated Myelopathy/Tropical Spastic

Padlan, E. A. A possible procedure for reducing the immunogenicity of antibody variable domains while preserving their ligand-binding properties. 1991 Mol Immunol 28:489-98.

Paraparesis: Towards Rational Targeted Therapy 2008 Neurol Clin. 2008 26: 781-785.

Orzaez, M., Gortat, A., Mondragon, L., Perez-Paya, E., Peptides and Peptide Mimics as Modulators of Apototic Pathways. 2009 Chem. Med. Chem. 4:146-160.

O'Shea, J. J., Targeting the Jak/STAT pathway for immunosuppression. 2004 Ann. Rheum. Dis. 63:(suppl II): ii67-71.

Paul, W. E., Pleiotropy and redundancy: T cell-derived lymphokines in the immune response. 1989 Cell 57:521-4.

Pesu M, Candotti F, Husa M, Hofmann S R, Notarangelo LD, and O'Shea J J. Jak3, severe combined immunodeficiency, and a new class of immunosuppressive drugs. 2005 Immunol. Rev. 203:127-142.

Pesu, M., Laurence, A., Kishore, N., Zwillich, S., Chan, G., O'Shea, J. J., Therapeutic targeting of Janus kinases. Immunol. 2008 Rev. 223:132-142.

Petukhova L., Duvic M., Hordinsky M., Norris D., Price V., Shimomura Y., Kim H., Singh P., Lee A., Chen W. V. et al. Genome-wide association study in alopecia areata implicates both innate and adaptive immunity. 2010 Nature 466:113-7.

Richmond J. M., Strassner J. P., Zapata L. Jr., Garg M., Riding R. L., Refat M. A., Fan X., Azzolino V., Tovar-Garza A., Tsurushita N. et al. Antibody blockade of IL-15 signaling has the potential to durably reverse vitiligo. Sci. Transl. Med. 2018 10:450.

Riechmann, L., Clark, M., Waldmann, H., and Winter, G. Reshaping human antibodies for therapy. 1988 Nature 332:323-7.

Rochman, Y., Spolski, R., Leonard, W. J., New Insights into the regulation of T cells by gamma c family cytokines. 2009 Nat. Rev. Immunol. 9:480-90.

Roguska, M. A., Pedersen, J. T., Keddy, C. A., Henry, A. H., Searle, S. J., Lambert, J. M., Goldmacher, V. S., Blättler, W. A., Rees, A. R., and Guild, B. C. Humanization of murine monoclonal antibodies through variable domain resurfacing. 1994 Proc Natl Acad Sci 91:969-73.

Saha, S. and Raghava, G. P. Prediction of continuous B-cell epitopes in an antigen using recurrent neural network. 2006 Proteins 65:40-8.

Sakaguchi, S., Yamaguchi, T., Nomura, T., Ono, M., Regulatory T cells and immune tolerance. 2008 Cell 133: 775-87.

Sato, N., Sabzevari, H., Fu, S., Ju, W., Bamford, R. N., Waldmann, T. A., and Tagaya, Y., Development of an IL-15-Autocrine CD8 T-cell Leukemia in IL-15 Transgenic mice requires the cis-expression of IL-15R alpha. 2011 117:4032-4040.

Schaller, J., Giese, T., Ladusch, M., Haustein, U. F., Interleukin-2 receptor expression and interleukin-2 production in bullous pemphigoid. 1990 Arch. Dermatol. Res. 282:223-226.

Shultz, L. D., Brehm, M. A., Garcia-Martinez, J. V., Greiner, D. L., Humanized mice for immune system investigation: progress, promise and challenges. 2012 12:786-798.

Sonntag, K., Eckert, F., Welker, C., Müller, H., Müller, F., Zips, D., Sipos, B., Klein, R., Blank, G., Feuchtinger, T. et al. Chronic graft-versus-host-disease in CD34(+)-humanized NSG mice is associated with human susceptibility HLA haplotypes for autoimmune diseases. 2015 J. Autoimmun. 62:55-66.

Studnicka, G. M., Soares, S., Better, M., Williams, R. E., Nadell, R., and Horwitz, A. H. Human-engineered monoclonal antibodies retain full specific binding activity by preserving non-CDR complementarity-modulating residues. 1994 Protein Eng 7:805-14.

Suarez-Farinas, M., Ungar, B., Noda, S., Shroff, A., Mansouri, Y., Fuentes-Duculan, J., Czernik, A., Zheng, X., Estrada, Y. D., Xu, H. et al. Alopecia areata profiling shows TH1, TH2, and IL-23 cytokine activation without parallel TH17/TH22 skewing. 2015 J. Allergy Clin. Immunol. 136:1277-1287.

Sugamura, K., Asao, H., Kondo, M., Tanaka, N., Ishii, N., Nakamura, M., Takeshita, T., The common gamma-chain for multiple cytokine receptors. 1995 Adv. Immunol. 59: 225-277.

Sugamura, K., Asao, H., Kondo, M., Tanaka, N., Ishii, N., Ohbo, K., Nakamura, M., Takeshita, T., The interleukin-2 receptor gamma chain: its role in the multiple cytokine receptor complexes and T cell development in XSCID. 1996 Annu. Rev. Immunol. 14:179-205.

Sushama S., Dixit N., Gautam R.K., Arora P., Khurana A., Anubhuti A., Cytokine profile (IL-2, IL-6, IL-17, IL-22, and TNF-alpha) in vitiligo-New insight into pathogenesis of disease. 2018 J. Cosmet. Dermatol. 00:1-5.

Tagaya, Y., Burton, J.D., Miyamoto, Y., Waldmann, TA., Identification of a novel receptor/signal transduction pathway for IL-15/T in mast cells. 1996 EMBO J. 15:4928-39.

Tagaya, Y., Memory CD8 T cells now join “Club 21”. 2010 J. Leuk. Biol. 87:13-15.

Takai, K., Sawasaki, T., and Endo. Y. The Wheat-Germ Cell-Free Expression System, 2010 Curr. Pharm. Biotechnol. 11:272-8.

Tanaka, T., et al., A novel monoclonal antibody against murine IL-2 receptor beta-chain. Characterization of receptor expression in normal lymphoid cells and EL-4 cells. 1991 J. Immunol. 147:2222-2228.

Takeshita, T., Asao, H., Ohtani, K., Ishii, N., Kumaki, S., Tanaka, N., Manukata, H., Nakamura, M., Sugamura, K., Cloning of the Gamma chain of the Human IL2 receptor. 1992 Science 257:379-382.

Tomimatsu, K. and Shirahata, S. Antigen-specific in vitro immunization: a source for human monoclonal antibodies. 2014 Methods Mol Biol 1060:297-307.

Vaughan, T. J., Williams, A. J., Pritchard, K., Osbourn, J. K., Pope, A. R., Earnshaw, J. C., McCafferty, J., Hodits, R. A., Wilton, J., and Johnson, K. S. Human Antibodies with Sub-nanomolar Affinities Isolated from a Large Non-immunized Phage Display Library. 1996 Nature 14:309-14.

Villadsen, L. S., Schuurman, J., Beurskens, F., Dam, T. N., Dagnaes-Hansen, F., Skov, L., Rygaard, J., Voorhorst-Ogink, M. M., Gerritsen, A. F., van Dijk, M. A., et al., Resolution of psoriasis upon blockade of IL-15 biological activity in a xenograft mouse model. 2003 J. Clin. Invest. 112:1571-1580.

Waldmann, T. A., Anti-Tac (daclizumab, Zenapax) in the treatment of leukemia, autoimmune diseases, and in the prevention of allograft rejection: a 25-year personal odyssey. 2007 J. Clin. Immunol. 27: 1-18.

Waldmann T. A. The biology of IL-15: implications for cancer therapy and the treatment of autoimmune disorders. 2013 J Investig Dermatol Symp Proc 16: S28-30.

Williams, D. G., Matthews, D.J., and Jones, T. “Humanising Antibodies by CDR Grafting.”, in: Antibody Engineering, 2010 eds R. Kontermann and S. Dübel (Berlin, Heidelberg: Springer).

Xing L., Dai Z., Jabbari A., Cerise J. E., Higgins C. A., Gong W., de Jong A., Harel S., DeStefano G. M., Rothman L. et al. Alopecia areata is driven by cytotoxic T lymphocytes and is reversed by JAK inhibition. 2014 Nat Med 9:1043-9.

Yao, B., Zhang, L., Liang, S., and Zhang, C. SVMTriP: A Method to Predict Antigenic Epitopes Using Support Vector Machine to Integrate Tri-Peptide Similarity and Propensity. 2012 PLOS One 7:e45152.

What is claimed is:

1. A composition, comprising:
a therapeutic compound in an amount sufficient to modulate signaling by at least one γc-cytokine family member, thereby inhibiting, ameliorating, reducing a severity of, treating, delaying the onset of, or preventing at least one alopecia related disorder; and
a pharmaceutically acceptable carrier;
wherein the therapeutic compound is a [symbol] c cytokine antagonist peptide;
wherein the [symbol] c cytokine antagonist peptide shares at least 90% identity with a peptide of SEQ ID NO: 1 (BNZ- [symbol] ).

2. The composition of claim 1, wherein the at least one alopecia related disorder is selected from the group consisting of alopecia, pemphigus, pemphigoid, psoriasis, vitiligo, graft-versus-host disease, and immune-mediated hair loss.

3. The composition of claim 1, wherein the γc cytokine antagonist peptide comprises 19 to 50 amino acids.

4. The composition of claim 1, wherein the γc cytokine antagonist peptide further comprises a conjugate at the N-termini, C-termini, side residues, or a combination thereof.

5. The compositions of claim 4, wherein the conjugate comprises one or more additional moieties selected from the group consisting of bovine serum albumin (BSA), albumin, Keyhole Limpet Hemocyanin (KLH), Fc region of IgG, a biological protein that functions as scaffold, an antibody against a cell-specific antigen, a receptor, a ligand, a metal ion, and Poly Ethylene Glycol (PEG).

6. The composition of claim 1, wherein the γc cytokine antagonist peptide further comprises a signal peptide.

7. The composition of claim 1, wherein the γc cytokine antagonist peptide comprises a sequence of SEQ ID NO: 1.

8. The composition of claim 1, wherein the γc cytokine antagonist peptide shares at least about 95% identity with a peptide of SEQ ID NO: 1.

9. The composition of claim 1, wherein the pharmaceutically acceptable carrier is formulated for topical, oral, or parenteral delivery.

10. A method of designing a γc-cytokine antagonist peptide configured to modulate and/or block signaling by at least one γc-cytokine family member that inhibits, ameliorates, reduces a severity of, treats, delays the onset of, or prevents at least one alopecia related disorder, the method comprising the steps of:
using a computer to obtain from an amino acid sequence database amino acid sequences of at least one a γc-cytokine family member; and
assembling a γc cytokine antagonist peptide based on a sequence of the at least one γc-cytokine family member;
wherein the γc cytokine antagonist peptide modulates and/or blocks signaling by the at least one γc-cytokine family member;
wherein the γc cytokine antagonist peptide shares at least about 90% identity with a peptide of SEQ ID NO: 1 (BNZ-γ).

11. The method of claim 10, wherein the γc cytokine antagonist peptide comprises 19 to 50 amino acids.

12. The method of claim 10, wherein the γc cytokine antagonist peptide further comprises a conjugate at the N-termini, C-termini, side residues, or a combination thereof.

13. The method of claim 10, wherein the γc cytokine antagonist peptide further comprises a signal peptide.

14. The method of claim 10, wherein the γc cytokine antagonist peptide shares at least about 95% identity with a peptide of SEQ ID NO: 1.

15. The method of claim 10, wherein the γc cytokine antagonist peptide comprises a sequence of SEQ ID NO: 1.

16. A kit for inhibiting, ameliorating, reducing a severity of, treating, delaying the onset of, or preventing at least one alopecia related disorder comprising:
a composition according to claim 1.

17. The kit of claim 16, wherein the at least one alopecia related disorder is selected from the group consisting of alopecia, pemphigus, pemphigoid, psoriasis, vitiligo, graft-versus-host disease, and immune-mediated hair loss.


SEQUENCE LISTING (46 sequences)

SEQ ID NO: 1 - protein, 19 aa, synthetic construct
  REGION 1..19: note = Synthetic
IKEFLQRFIHIVQSIINTS

SEQ ID NO: 2 - skipped

SEQ ID NO: 3 - protein, 19 aa, synthetic construct
  REGION 1..19: note = Synthetic
IKEFLQSFVHIVQMFINTS

SEQ ID NO: 4 - protein, 20 aa, synthetic construct
  REGION 1..20: note = Synthetic
IVEFLNRWITFCQSIISTLT

SEQ ID NO: 5 - protein, 21 aa, synthetic construct
  REGION 1..21: note = Synthetic
PKEFLERFKSLLQKMIHQHLS

SEQ ID NO: 6 - protein, 21 aa, synthetic construct
  REGION 1..21: note = Synthetic
LENFLERLKTIMREKYSKCSS

SEQ ID NO: 7 - protein, 21 aa, synthetic construct [sequence omitted]

SEQ ID NO: 8 - protein, 20 aa, synthetic construct
  REGION 1..20: note = Synthetic
DLCFLKRLLQEIKTCWNKIL

SEQ ID NO: 9 - skipped

SEQ ID NO: 10 - protein, 19 aa, synthetic construct
  REGION 1..19: note = Synthetic
  SITE 1: note = MISC_FEATURE - X = Any amino acid
  SITE 2: note = MISC_FEATURE - X = Any amino acid
  SITE 3: note = MISC_FEATURE - X = D or E
  SITE 6: note = MISC_FEATURE - X = E or Q or N or polar amino acid
  SITE 7: note = MISC_FEATURE - X = S or R or polar amino acid
  SITE 8: note = MISC_FEATURE - X = Non-polar amino acid
  SITE 9: note = MISC_FEATURE - X = I or K or non-polar amino acid
  SITE 10: note = MISC_FEATURE - X = Any amino acid
  SITE 11: note = MISC_FEATURE - X = L or I or aliphatic amino acid
  SITE 12: note = MISC_FEATURE - X = Non-polar amino acid
  SITE 14: note = MISC_FEATURE - X = Charged amino acid
  SITE 15: note = MISC_FEATURE - X = Any amino acid
  SITE 17: note = MISC_FEATURE - X = Any amino acid
XXXFLXXXXXXXQXXIXTS

SEQ ID NO: 11 - protein, 20 aa, synthetic construct
  REGION 1..20: note = Synthetic
  SITE 1: note = MISC_FEATURE - S-pentenylalanine; Linked to S-pentenylalanine at position 5
  SITE 5: note = MISC_FEATURE - S-pentenylalanine; Linked to S-pentenylalanine at position 1
AIKEALQRFIHIVQSIINTS

SEQ ID NO: 12 - protein, 19 aa, synthetic construct
  REGION 1..19: note = Synthetic
  SITE 8: note = MISC_FEATURE - S-pentenylalanine; Linked to S-pentenylalanine at position 12
  SITE 12: note = MISC_FEATURE - S-pentenylalanine; Linked to S-pentenylalanine at position 8
IKEFLQRAIHIAQSIINTS

SEQ ID NO: 13 - protein, 19 aa, synthetic construct
  REGION 1..19: note = Synthetic
  SITE 8: note = MISC_FEATURE - R-octenylalanine; Linked to S-pentenylalanine at position 15
  SITE 15: note = MISC_FEATURE - S-pentenylalanine; Linked to R-octenylalanine at position 8
IKEFLQRAIHIVQSAINTS

SEQ ID NO: 14 - protein, 19 aa, synthetic construct
  REGION 1..19: note = Synthetic
  SITE 12: note = MISC_FEATURE - S-pentenylalanine; Linked to S-pentenylalanine at position 16
  SITE 16: note = MISC_FEATURE - S-pentenylalanine; Linked to S-pentenylalanine at position 12
IKEFLQRFIHIAQSIANTS

SEQ ID NO: 15 - protein, 19 aa, synthetic construct
  REGION 1..19: note = Synthetic
  SITE 12: note = MISC_FEATURE - R-octenylalanine; Linked to S-pentenylalanine at position 19
  SITE 19: note = MISC_FEATURE - S-pentenylalanine; Linked to R-octenylalanine at position 12
IKEFLQRFIHIAQSIINTA

SEQ ID NO: 16 - protein, 20 aa, synthetic construct
  REGION 1..20: note = Synthetic
  SITE 1: note = MISC_FEATURE - S-pentenylalanine; Linked to S-pentenylalanine at position 5
  SITE 5: note = MISC_FEATURE - S-pentenylalanine; Linked to S-pentenylalanine at position 1
  SITE 9: note = MISC_FEATURE - S-pentenylalanine; Linked to S-pentenylalanine at position 13
  SITE 13: note = MISC_FEATURE - S-pentenylalanine; Linked to S-pentenylalanine at position 9
AIKEALQRAIHIAQSIINTS

SEQ ID NO: 17 - protein, 20 aa, synthetic construct
  REGION 1..20: note = Synthetic
  SITE 1: note = MISC_FEATURE - S-pentenylalanine; Linked to S-pentenylalanine at position 5
  SITE 5: note = MISC_FEATURE - S-pentenylalanine; Linked to S-pentenylalanine at position 1
  SITE 9: note = MISC_FEATURE - R-octenylalanine; Linked to S-pentenylalanine at position 16
  SITE 16: note = MISC_FEATURE - S-pentenylalanine; Linked to R-octenylalanine at position 8
AIKEALQRAIHIVQSAINTS

SEQ ID NO: 18 - protein, 20 aa, synthetic construct
  REGION 1..20: note = Synthetic
  SITE 1: note = MISC_FEATURE - S-pentenylalanine; Linked to S-pentenylalanine at position 5
  SITE 5: note = MISC_FEATURE - S-pentenylalanine; Linked to S-pentenylalanine at position 1
  SITE 13: note = MISC_FEATURE - S-pentenylalanine; Linked to S-pentenylalanine at position 17
  SITE 17: note = MISC_FEATURE - S-pentenylalanine; Linked to S-pentenylalanine at position 13
AIKEALQRFIHIAQSIANTS

SEQ ID NO: 19 - protein, 20 aa, synthetic construct
  REGION 1..20: note = Synthetic
  SITE 1: note = MISC_FEATURE - S-pentenylalanine; Linked to S-pentenylalanine at position 5
  SITE 5: note = MISC_FEATURE - S-pentenylalanine; Linked to S-pentenylalanine at position 1
  SITE 13: note = MISC_FEATURE - R-octenylalanine; Linked to S-pentenylalanine at position 20
  SITE 20: note = MISC_FEATURE - S-pentenylalanine; Linked to R-octenylalanine at position 13
AIKEALQRFIHIAQSIINTA

SEQ ID NO: 20 - protein, 20 aa, synthetic construct
  REGION 1..20: note = Synthetic
  SITE 1: note = MISC_FEATURE - S-pentenylalanine; Linked to S-pentenylalanine at position 5
  SITE 5: note = MISC_FEATURE - S-pentenylalanine; Linked to S-pentenylalanine at position 1
  SITE 9: note = MISC_FEATURE - S-pentenylalanine; Linked to S-pentenylalanine at position 13
  SITE 13: note = MISC_FEATURE - S-pentenylalanine; Linked to S-pentenylalanine at position 9
  SITE 18: note = MISC_FEATURE - D stereochemical configuration
  SITE 19: note = MISC_FEATURE - D stereochemical configuration
  SITE 20: note = MISC_FEATURE - D stereochemical configuration
AIKEALQRAIHIAQSIINTS

SEQ ID NO: 21 - protein, 20 aa, synthetic construct
  REGION 1..20: note = Synthetic
  SITE 1: note = MISC_FEATURE - S-pentenylalanine; Linked to S-pentenylalanine at position 5
  SITE 5: note = MISC_FEATURE - S-pentenylalanine; Linked to S-pentenylalanine at position 1
  SITE 9: note = MISC_FEATURE - R-octenylalanine; Linked to S-pentenylalanine at position 16
  SITE 16: note = MISC_FEATURE - S-pentenylalanine; Linked to R-octenylalanine at position 9
  SITE 18: note = MISC_FEATURE - D stereochemical configuration
  SITE 19: note = MISC_FEATURE - D stereochemical configuration
  SITE 20: note = MISC_FEATURE - D stereochemical configuration
AIKEALQRAIHIVQSAINTS

SEQ ID NO: 22 - protein, 20 aa, synthetic construct
  REGION 1..20: note = Synthetic
  SITE 1: note = MISC_FEATURE - S-pentenylalanine; Linked to S-pentenylalanine at position 5
  SITE 5: note = MISC_FEATURE - S-pentenylalanine; Linked to S-pentenylalanine at position 1
  SITE 13: note = MISC_FEATURE - S-pentenylalanine; Linked to S-pentenylalanine at position 17
  SITE 17: note = MISC_FEATURE - S-pentenylalanine; Linked to S-pentenylalanine at position 13
  SITE 18: note = MISC_FEATURE - D stereochemical configuration
  SITE 19: note = MISC_FEATURE - D stereochemical configuration
  SITE 20: note = MISC_FEATURE - D stereochemical configuration
AIKEALQRFIHIAQSIANTS

SEQ ID NO: 23-24 - skipped (2 entries)

SEQ ID NO: 25 - DNA, 708 bp, Homo sapiens
  source 1..708: mol_type = other DNA
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtctaa

SEQ ID NO: 26 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 27 - DNA, 732 bp, Homo sapiens
  source 1..732: mol_type = other DNA
atgcggccgcggctgtggctcctcttggccgcgcagctgacagttctccatggcaactcagtcctccagcagacccctgcatacataaaggtgcaaaccaacaagatggtgatgctgtcctgcgaggctaaaatctccctcagtaacatgcgcatctactggctgagacagcgccaggcaccgagcagtgacagtcaccacgagttcctggccctctgggattccgcaaaagggactatccacggtgaagaggtggaacaggagaagatagctgtgtttcgggatgcaagccggttcattctcaatctcacaagcgtgaagccggaagacagtggcatctacttctgcatgatcgtcgggagccccgagctgaccttcgggaagggaactcagctgagtgtggttgatttccttcccaccactgcccagcccaccaagaagtccaccctcaagaagagagtgtgccggttacccaggccagagacccagaagggcccactttgtagccccatcacccttggcctgctggtggctggcgtcctggttctgctggtttccctgggagtggccatccacctgtgctgccggcggaggagagcccggcttcgtttcatgaaacagcctcaaggggaaggtatatcaggaacctttgtcccccaatgcctgcatggatactacagcaatactacaacctcacagaagctgcttaacccatggatcctgaaaacatag

SEQ ID NO: 28 - protein, 243 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 29 - DNA, 462 bp, Homo sapiens
  source 1..462: mol_type = other DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 30 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 31 - DNA, 489 bp, Homo sapiens
  source 1..489: mol_type = other DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 32 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 33 - DNA, 702 bp, Homo sapiens
  source 1..702: mol_type = other DNA
atggataaccaaggagtaatctactcagacctgaatctgcccccaaacccaaagaggcagcaacgaaaacctaaaggcaataaaagctccattttagcaactgaacaggaaataacctatgcggaattaaaccttcaaaaagcttctcaggattttcaagggaatgacaaaacctatcactgcaaagatttaccatcagctccagagaagctcattgttgggatcctgggaattatctgtcttatcttaatggcctctgtggtaacgatagttgttattccctctacattaatacagaggcacaacaattcttccctgaatacaagaactcagaaagcacgtcattgtggccattgtcctgaggagtggattacatattccaacagttgttactacattggtaaggaaagaagaacttgggaagagagtttgctggcctgtacttcgaagaactccagtctgctttctatagataatgaagaagaaatgaaatttctgtccatcatttcaccatcctcatggattggtgtgtttcgtaacagcagtcatcatccatgggtgacaatgaatggtttggctttcaaacatgagataaaagactcagataatgctgaacttaactgtgcagtgctacaagtaaatcgacttaaatcagcccagtgtggatcttcaataatatatcattgtaagcataagctttag

SEQ ID NO: 34 - protein, 233 aa, Homo sapiens
MDNQGVIYSDLNLPPNPKRQQRKPKGNKSSILATEQEITYAELNLQKASQDFQGNDKTYHCKDLPSAPEKLIVGILGIICLILMASVVTIVVIPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 35 - DNA, 648 bp, Homo sapiens
  source 1..648: mol_type = other DNA
atggataaccaaggagtaatctactcagacctgaatctgcccccaaacccaaagaggcagcaacgaaaacctaaaggcaataaaagctccattttagcaactgaacaggaaataacctatgcggaattaaaccttcaaaaagcttctcaggattttcaagggaatgacaaaacctatcactgcaaagatttaccatcagctccagagaagctcattgttgggatcctgggaattatctgtcttatcttaatggcctctgtggtaacgatagttgttattccctcacgtcattgtggccattgtcctgaggagtggattacatattccaacagttgttactacattggtaaggaaagaagaacttgggaagagagtttgctggcctgtacttcgaagaactccagtctgctttctatagataatgaagaagaaatgaaatttctgtccatcatttcaccatcctcatggattggtgtgtttcgtaacagcagtcatcatccatgggtgacaatgaatggtttggctttcaaacatgagataaaagactcagataatgctgaacttaactgtgcagtgctacaagtaaatcgacttaaatcagcccagtgtggatcttcaataatatatcattgtaagcataagctttag

SEQ ID NO: 36 - protein, 215 aa, Homo sapiens
MDNQGVIYSDLNLPPNPKRQQRKPKGNKSSILATEQEITYAELNLQKASQDFQGNDKTYHCKDLPSAPEKLIVGILGIICLILMASVVTIVVIPSRHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 37 - DNA, 696 bp, Homo sapiens
  source 1..696: mol_type = other DNA
atgaataaacaaagaggaaccttctcagaagtgagtctggcccaggacccaaagcggcagcaaaggaaacctaaaggcaataaaagctccatttcaggaaccgaacaggaaatattccaagtagaattaaatcttcaaaatccttccctgaatcatcaagggattgataaaatatatgactgccaaggtttactgccacctccagagaagctcactgccgaggtcctaggaatcatttgcattgtcctgatggccactgtgttaaaaacaatagttcttattcctttcctggagcagaacaatttttccccgaatacaagaacgcagaaagcacgtcattgtggccattgtcctgaggagtggattacatattccaacagttgttattacattggtaaggaaagaagaacttgggaagagagtttgctggcctgtacttcgaagaactccagtctgctttctatagataatgaagaagaaatgaaatttctggccagcattttaccttcctcatggattggtgtgtttcgtaacagcagtcatcatccatgggtgacaataaatggtttggctttcaaacataagataaaagactcagataatgctgaacttaactgtgcagtgctacaagtaaatcgacttaaatcagcccagtgtggatcttcaatgatatatcattgtaagcataagctttag

SEQ ID NO: 38 - protein, 231 aa, Homo sapiens
MNKQRGTFSEVSLAQDPKRQQRKPKGNKSSISGTEQEIFQVELNLQNPSLNHQGIDKIYDCQGLLPPPEKLTAEVLGIICIVLMATVLKTIVLIPFLEQNNFSPNTRTQKARHCGHCPEEWITYSNSCYYIGKERRTWEESLLACTSKNSSLLSIDNEEEMKFLASILPSSWIGVFRNSSHHPWVTINGLAFKHKIKDSDNAELNCAVLQVNRLKSAQCGSSMIYHCKHKL

SEQ ID NO: 39 - DNA, 651 bp, Homo sapiens
  source 1..651: mol_type = other DNA
atggggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatctccattttttttctgctgcttcatcgctgtagccatgggaatccgtttcattattatggtaacaatatggagtgctgtattcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgtaa

SEQ ID NO: 40 - protein, 216 aa, Homo sapiens
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 41 - DNA, 723 bp, Homo sapiens
  source 1..723: mol_type = other DNA
atgagtaaacaaagaggaaccttctcagaagtgagtctggcccaggacccaaagtggcagcaaaggaaacctaaaggcaataaaagctccatttcaggaaccgaacaggaaatattccaagtagaattaaaccttcaaaatgcttctctgaatcatcaagggattgataaaatatatgactgccaaggtttactgccacctccagaaaagctcactgccgaggtcctaggaatcatttgcattgtcctgatggccactgtgttaaaaacaatagttcttattcctttcctggagcagaacaattcttccccgaatgcaagaacccagaaagcacgtcattgtggccattgtcctgaggagtggattacatattccaacagttgttattacattggtaaggaaagaagaacttgggaagagagtttgcaggcctgtgcttcaaagaactcttctagtctgctttgtatagataatgaagaagaaatgaaatttctggccagcattttaccttcctcatggattggtgtgtttcgtaacagcagtcatcatccatgggtgacaataaatggtttggctttcaaacatgagataaaagactcagatcatgctgaacgtaactgtgcaatgctacatgtacgtggacttatatcagaccagtgtggatcttcaagaatcattagacggggtttcatcatgttgaccaggctggtcttgaactcctga

SEQ ID NO: 42 - protein, 240 aa, Homo sapiens
MSKQRGTFSEVSLAQDPKWQQRKPKGNKSSISGTEQEIFQVELNLQNASLNHQGIDKIYDCQGLLPPPEKLTAEVLGIICIVLMATVLKTIVLIPFLEQNNSSPNARTQKARHCGHCPEEWITYSNSCYYIGKERRTWEESLQACASKNSSSLLCIDNEEEMKFLASILPSSWIGVFRNSSHHPWVTINGLAFKHEIKDSDHAERNCAMLHVRGLISDQCGSSRIIRRGFIMLTRLVLNS

SEQ ID NO: 43 - DNA, 477 bp, Homo sapiens
  source 1..477: mol_type = other DNA
atgaataaacaaagaggaacctactcagaagtgagtctggcccaggacccaaagaggcagcaaaggaaacttaagggcaataaaatctccatttcaggaaccaaacaggaaatattccaagtagaattaaaccttcaaaatgcttcttcggatcatcaagggaatgacaagacatatcactgcaaaggtttactgccacctccagagaagctcactgctgaggtcctaggaatcatttgcattgtcctgatggccactgtgttaaaaacaatagttcttattccttgtattggagtactggagcagaacaatttttccctgaatagaagaatgcagaaagcacgtcattgtggccattgtcctgaggagtggattacatattccaacagttgttattacattggtaaggaaagaagaacttgggaagaaagagtttgctggcctgtgcttcgaagaactctgatctgctttctatag

SEQ ID NO: 44 - protein, 158 aa, Homo sapiens
MNKQRGTYSEVSLAQDPKRQQRKLKGNKISISGTKQEIFQVELNLQNASSDHQGNDKTYHCKGLLPPPEKLTAEVLGIICIVLMATVLKTIVLIPCIGVLEQNNFSLNRRMQKARHCGHCPEEWITYSNSCYYIGKERRTWEERVCWPVLRRTLICFL

SEQ ID NO: 45 - DNA, 774 bp, Homo sapiens
  source 1..774: mol_type = other DNA
atgagtaaacaaagaggaaccttctcagaagtgagtctggcccaggacccaaagtggcagcaaaggaaacctaaaggcaataaaagctccatttcaggaaccgaacaggaaatattccaagtagaattaaaccttcaaaatgcttctctgaatcatcaagggattgataaaatatatgactgccaaggtttactgccacctccagaaaagctcactgccgaggtcctaggaatcatttgcattgtcctgatggccactgtgttaaaaacaatagttcttattcctttcctggagcagaacaattcttccccgaatgcaagaacccagaaagcacgtcattgtggccattgtcctgaggagtggattacatattccaacagttgttattacattggtaaggaaagaagaacttgggaagagagtttgcaggcctgtgcttcaaagaactcttctagtctgctttgtatagataatgaagaagaaatgaaatttctggccagcattttaccttcctcatggattggtgtgtttcgtaacagcagtcatcatccatgggtgacaataaatggtttggctttcaaacatgagataaaagactcagatcatgctgaacgtaactgtgcaatgctacatgtacgtggacttatatcagaccagtgtggatcttcaagaatcattgtgagcataagctttagaattaaagcgcttgagcttgcagtgcatcagataaaattttatatttgttcaaacagaaatgatattatgattgcataa

SEQ ID NO: 46 - protein, 257 aa, Homo sapiens
MSKQRGTFSEVSLAQDPKWQQRKPKGNKSSISGTEQEIFQVELNLQNASLNHQGIDKIYDCQGLLPPPEKLTAEVLGIICIVLMATVLKTIVLIPFLEQNNSSPNARTQKARHCGHCPEEWITYSNSCYYIGKERRTWEESLQACASKNSSSLLCIDNEEEMKFLASILPSSWIGVFRNSSHHPWVTINGLAFKHEIKDSDHAERNCAMLHVRGLISDQCGSSRIIVSISFRIKALELAVHQIKFYICSNRNDIMIA